(19) United States
(12) Patent Application Publication
(10) Pub. No.: US 2026/0250657 A1
(43) Pub. Date: Aug. 27, 2026

(54) METHODS AND COMPOSITIONS FOR REGULATING GENE EXPRESSION

(71) Applicant: THE UNIVERSITY OF CHICAGO, Chicago, IL (US)

(72) Inventors: Chuan HE, Chicago, IL (US); Hui-Lung SUN, Chicago, IL (US); Yan-Ming CHEN, Chicago, IL (US)

(73) Assignee: THE UNIVERSITY OF CHICAGO, Chicago, IL (US)

(21) Appl. No.: 19/489,713

(22) PCT Filed: Jun. 14, 2024
(86) PCT No.: PCT/US2024/034147

§ 371 Date: Dec. 3, 2025

Related U.S. Application Data
(60) Provisional application No. 63/508,766, filed on Jun. 16, 2023.

(51) Int. Cl. C12N 9/90; A61K 38/00; C12N 9/22; C12N 15/11

(52) U.S. Cl. CPC C12N 9/90; C12N 9/226; C12N 15/11; C12Y 504/99025; A61K 38/00; C07K 2319/00; C12N 2310/20

(57) ABSTRACT

Aspects of the present disclosure are directed to at least methods and compositions for site-specific targeting or RNA and/or pseudouridine installation in RNA, such as mRNA. Provided targeting and/or pseudouridine installation can increase stability of RNA, increase mRNA translation, and/or fine-tune protein expression. Also disclosed herein are compositions, methods, and kits suitable for therapeutic application of site-specific targeting of RNA and/or pseudouridine installation in RNA.

### CROSS REFERENCE TO RELATED APPLICATIONS

The present invention claims priority to U.S. Provisional Application Ser. No. 63/508,766 filed on Jun. 16, 2023, which is incorporated by reference herein in its entirety.

### STATEMENT OF GOVERNMENT SUPPORT

This invention was made with government support under HG008935 awarded by the National Institutes of Health. The government has certain rights in the invention.

### SEQUENCE LISTING

The instant application contains a Sequence Listing which has been submitted in ST26 format and is hereby incorporated by reference in its entirety. Said ST26 copy, created on Jun. 14, 2024, is named ARCD_P0805WO_Sequence_Listing.xml and is 63,824 bytes in size.

### BACKGROUND

### I. Field of the Invention

Aspects of this invention relate to at least the field of molecular biology. More particularly, aspects concern at least methods for modifying, detecting, mapping, evaluating, and/or installing pseudouridine within a nucleic acid molecule.

### II. Background

Peudouridine (Ψ) is the most abundant RNA modification with an estimated T/U ratio of 7-9%, with most Ψ in ribosome RNA and transfer RNA. The ratio of Ψ/U in mammalian mRNA has also been observed to be about 0.2-0.6%, a frequency comparable to that of m⁶A (PMID: 26075521, PMID: 23177736, each of which are incorporated herein by reference in their entirety for the purposes described herein). Ψ can be catalyzed by stand-alone pseudouridine synthases (PUSs) or a snoRNA guided-RNA complex (snoRNA as the guide RNA recognized by DKC1, dyskerin pseudouridine synthase 1). In the human genome, 13 proteins have been annotated as putative PUS (PMID: 34468991, which is incorporated herein by reference in its entirety for the purposes described herein). The PUSs have been shown to target different RNA species to perform distinct functions, including transcriptome-wide regulation of translation (rRNA and tRNA), splicing, RNA stability, protein-RNA interaction, and response to cellular environments (PMID: 3546171, which is incorporated herein by reference in its entirety for the purposes described herein). The dysregulation or depletion of individual PUSs has been purported to cause defects in RNA metabolism and leads to cellular phenotypes or even disease (PMID: 30526862, PMID: 15108122, PMID: 35121864, each of which are incorporated herein by reference in their entirety for the purposes described herein).

Recently, the Inventors developed bisulfite-induced deletion sequencing (BID-seq) to profile the Ψ transcriptome landscape in single nucleotide resolution. BID-seq revealed abundant Ψ sites in various mouse tissue mRNA (PMID: 36302989, which is incorporated herein by reference in its entirety for the purposes described herein). Methods, compositions, and kits concerning BID-seq can be found in International Patent Application publication WO 2022/232795 A1, filed on Apr. 27, 2022, which is incorporated herein in its entirety. The BID-seq method can facilitate single nucleotide resolution interrogation of a transcriptome, facilitating the assignment of individual Ψ to distinct pseudouridine synthases. Transcriptome features of Ψ suggest that installing Ψ within specific mRNAs may alter protein expression without altering the genome.

There exists a need for methods and compositions for directed Ψ modification for commercial use, such as development of targeted therapeutics applications.

### SUMMARY OF THE INVENTION

The present disclosure addresses certain needs by providing at least methods, compositions, and kits for targeted Ψ installation within RNA species, such as messenger RNA (mRNA).

In some aspects, provided herein are compositions and methods for modulation of protein levels (e.g., increasing protein levels) associated with (e.g., encoded by) a target gene by introducing Ψ to the encoding mRNA, for example in specific rare codons and/or pre-mature stop codons. In some aspects, such Ψ installation can be via mRNA contact with a programmable dCas13d-TRUB1 system as described herein. In some aspects, a dCas13d-TRUB1 system comprises a truncated TRUB1 protein consisting essentially of the catalytic domain of the TRUB1 protein.

In some aspects, provided herein are compositions and methods for modulation of protein levels (e.g., increasing protein levels) associated with (e.g., encoded by) a target gene by contacting an encoding mRNA at rare codons with an engineered snoRNA and/or a fraction of an engineered snoRNA. In some aspects, the engineered snoRNA may recruit native (e.g., endogenous) DKC1 and/or heterologously overexpressed DKC1 for site-specific pseudouridine installation. In some aspects, a DKC1 is DKC1 isoform 3. In some aspects, a DKC1 is an endogenous cytoplasmic DKC1 isoform. In some aspects, a DKC1 is an endogenous cytoplasmic DKC1 isoform 3. In some aspects, a DKC1 is a heterologous cytoplasmic DKC1 isoform. In some aspects, a DKC1 is a heterologous cytoplasmic DKC1 isoform 3.

In some aspects, provided herein are compositions and method for modulating (e.g., increasing or decreasing protein levels) protein levels of a target gene by creating a modification that changes Uridine to Ψ at specific sites and/or on specific mRNA transcripts. In some aspects, provided herein are compositions and method for modulating (e.g., increasing or decreasing protein levels) protein levels of a target gene by contacting a pseudouridine synthase to specific sites and/or on specific mRNA transcripts.

In particular aspects, provided herein are compositions comprising, a pseudouridine synthase protein linked to a targeting protein. In some aspects, the targeting protein comprises an RNA-guided protein. In some aspects, the targeting protein comprises a clustered regularly interspaced short palindromic repeats (CRISPR) Associated (Cas) Protein. In some aspects, the Cas protein endonuclease enzymatic activity is non-functional. In some aspects, wherein the Cas protein comprises a dCas13d protein. In some aspects, the Cas protein comprises an amino acid sequence identity at least 80%, 85%, 90%, 95%, 99%, or 100% identical to SEQ ID NO: 20. In some aspects, the pseudouridine synthase protein comprises PUS1, PUS3, PUS4 (TRUB1), PUS7, PUS10, and/or RPUSD2. In some aspects, the pseudouridine synthase protein comprises an amino acid sequence at least 80%, 85%, 90%, 95%, 99%, or 100% identical to one or more of SEQ ID NOs: 2, 4, 6, 8, 10, 12, 14, 16, or 18. In some aspects, the pseudouridine synthase protein comprises, consists essentially of, or consists of a TRUB1 protein and/or catalytic portion thereof. In some aspects, the pseudouridine synthase protein consists of or consists essentially of a protein comprising an amino acid sequence at least 80%, 85%, 90%, 95%, 99%, or 100% identical to SEQ ID NO: 6, 8, 10, or 12. In some aspects, compositions further comprise one or more guide RNA (gRNA) molecules that target a messenger RNA (mRNA) molecule associated with (e.g., encoded by) a target gene.

In some aspects, also provided herein are compositions comprising one or more guide RNA (gRNA) molecules that target a messenger RNA (mRNA) molecule associated with (e.g., encoded by) a target gene, for use with a pseudouridine synthase, such as a pseudouridine synthase comprising composition as disclosed herein. In some aspects, a targeting RNA (e.g., a gRNA) targets a gene associated with diseases and/or disorders characterized by haploinsufficiency, gene downregulation, and/or gene translation inhibiting polymorphisms. In some aspects, the gene translation inhibiting polymorphisms comprise one or more of a premature stop codon, rare codon, and/or missense codon mutation. In some aspects, the targeting RNA (e.g., gRNA) targets a gene comprising one or more rare codons. In some aspects, the rare codon is ATA, TTT, CAT, TTA, AAT, or TAT. In some aspects, the rare codon is ATA. In some aspects, the gene comprises more than one rare codon. In some aspects, the gene comprises more than one different rare codon. In some aspects, the targeting RNA (e.g., gRNA) molecule targets a transcript associated with (e.g., encoded by) any of genes KRAS, p53, DMD, HBB, IDUA NF1, CDC6, AMFR, SCP2, ERH, CFTR, GLMN, POT1, TBK1, KRIT1, NBN, FANCB, DSC2, LIG4, ATP2C1, PGAP1, RB1, MSH2, RASA1, DSG1, KIF11, CHL1, RAD50, ATP7A, PHIP, SI, APC, ATM, and/or BRCA2. In some aspects, the targeting RNA (e.g., gRNA) molecule comprises a polynucleotide sequence at least 85%, 90%, 95%, or 100% identical to any one or more of SEQ ID NOs: 21-28.

Also provided herein are compositions comprising a pseudouridine synthase protein linked to a targeting protein, and complexed or otherwise associated with a targeting RNA (e.g., a guide RNA) described herein.

Also provided herein are methods of modulating translation of a target mRNA comprising, contacting one or more site-specific rare codons in the target mRNA with a) one or more site-specific targeting elements, and b) one or more of a pseudouridine synthase protein and/or one or more box H/ACA small nucleolar ribonucleoprotein (H/ACA snoRNP) complex components. In some aspects, a pseudouridine is installed on the target mRNA. In some aspects, b) comprises a pseudouridine synthase protein fused to a targeting protein. In some aspects, a) comprises a targeting protein and/or polypeptide. In some aspects, a) comprises an RNA-guided protein. In some aspects, the targeting element comprises a clustered regularly interspaced short palindromic repeats (CRISPR) Associated (Cas) Protein. In some aspects, the Cas protein endonuclease enzymatic activity is non-functional. In some aspects, the Cas protein comprises a dCas13d protein. In some aspects, the Cas protein comprises a sequence identity at least 80%, 85%, 90%, 95%, 99%, or 100% identical to SEQ ID NO: 20. In some aspects, the pseudouridine synthase protein comprises PUS1, PUS3, PUS4 (TRUB1), PUS7, PUS10, and/or RPUSD2. In some aspects, the pseudouridine synthase protein comprises a sequence at least 80%, 85%, 90%, 95%, 99%, or 100% identical to one or more of SEQ ID NOs: 2, 4, 6, 8, 10, 12, 14, 16, or 18. In some aspects, the pseudouridine synthase protein comprises, consists essentially of, or consists of a TRUB1 protein and/or catalytic portion thereof. In some aspects, the pseudouridine synthase protein consists of or consists essentially of a protein comprising a sequence at least 80%, 85%, 90%, 95%, 99%, or 100% identical to SEQ ID NOs: 6, 8, 10, or 12. In some aspects, the pseudouridine synthase protein consists of or consists essentially of a protein comprising a sequence at least 80%, 85%, 90%, 95%, 99%, or 100% identical to SEQ ID NO: 12. In some aspects, the targeting element comprises one or more guide RNA (gRNA) molecules. In some aspects, the gRNA targets a gene associated with diseases and/or disorders characterized by haploinsufficiency, gene downregulation, and/or gene translation inhibiting polymorphisms. In some aspects, the gRNA targets a gene comprising one or more rare codons. In some aspects, the gene comprises more than one different rare codon. In some aspects, the rare codon is ATA, TTT, CAT, TTA, AAT, or TAT. In some aspects, the rare codon is ATA. In some aspects, the target mRNA comprises more than one rare codons. In some aspects, the target mRNA is a transcript associated with (e.g., encoded by) any of genes KRAS, p53, DMD, HBB, IDUA NF1, CDC6, AMFR, SCP2, ERH, CFTR, GLMN, POT1, TBK1, KRIT1, NBN, FANCB, DSC2, LIG4, ATP2C1, PGAP1, RB1, MSH2, RASA1, DSG1, KIF11, CHL1, RAD50, ATP7A, PHIP, SI, APC, ATM, and/or BRCA2. In some aspects, the targeting element comprises a polynucleotide sequence at least 85%, 90%, 95%, or 100% identical to any one or more of SEQ ID NOs: 21-28. In some aspects, the target mRNA is a transcript associated with (e.g., encoded by) the KRAS gene. In some aspects, the target mRNA is a transcript associated with (e.g., encoded by) the CTFR gene. In some aspects, the targeting element comprises a polynucleotide sequence at least 85%, 90%, 95%, or 100% identical to any one or more of SEQ ID NOs: 25-28. In some aspects, the targeting element comprises a polynucleotide sequence at least 85%, 90%, 95%, or 100% identical to any one or more of SEQ ID NOs: 27. In some aspects, the pseudouridine is installed in KRAS mRNA and increases KRAS protein levels. In some aspects, the targeting element comprises an engineered sno-RNA. In some aspects, the engineered sno-RNA targets a transcript associated with (e.g., encoded by) any of genes KRAS, p53, DMD, HBB, IDUA NF1, CDC6, AMFR, SCP2, ERH, CFTR, GLMN, POT1, TBK1, KRIT1, NBN, FANCB, DSC2, LIG4, ATP2C1, PGAP1, RB1, MSH2, RASA1, DSG1, KIF11, CHL1, RAD50, ATP7A, PHIP, SI, APC, ATM, and/or BRCA2. In some aspects, the engineered sno-RNA targets a transcript associated with (e.g., encoded by) KRAS. In some aspects, the engineered sno-RNA comprises, consists essentially of, or consists of a polynucleotide sequence at least 85%, 90%, 95%, or 100% identical to any one or more of SEQ ID NOs: 37-45.

Also provided herein are methods of treating a disease and/or disorder in an individual comprising, providing the individual in need thereof with a composition described herein. Also provided herein are methods of treating a disease and/or disorder in an individual comprising, performing the method of modulating translation levels of mRNA described herein. In some aspects, the disease and/or disorder is cancer. In some aspects, the disease and/or disorder is characterized by haploinsufficiency.

Certain aspects of the present invention are characterized through the following enumerated aspects.

Aspect 1 is a composition comprising, a pseudouridine synthase protein linked to a targeting protein.

Aspect 2 is the composition of aspect 1, wherein the targeting protein comprises an RNA-guided protein.

Aspect 3 is the composition of aspect 1 or 2, wherein the targeting protein comprises a clustered regularly interspaced short palindromic repeats (CRISPR) Associated (Cas) Protein

Aspect 4 is the composition of aspect 3, wherein the Cas protein endonuclease enzymatic activity is non-functional.

Aspect 5 is the composition of aspects 3 or 4, wherein the Cas protein comprises a dCas13d protein.

Aspect 6 is the composition of any one of aspects 3 to 5, wherein the Cas protein comprises an amino acid sequence identity at least 80%, 85%, 90%, 95%, 99%, or 100% identical to SEQ ID NO: 20.

Aspect 7 is the composition of any one of aspects 1 to 6, wherein the pseudouridine synthase protein comprises PUS4 (TRUB1), PUS1, PUS3, PUS7, PUS10, and/or RPUSD2.

Aspect 8 is the composition of any one of aspects 1 to 7, wherein the pseudouridine synthase protein comprises an amino acid sequence at least 80%, 85%, 90%, 95%, 99%, or 100% identical to one or more of SEQ ID NOs: 6, 8, 10, 12, 2, 4, 14, 16, or 18.

Aspect 9 is the composition of any one of aspects 1 to 8, wherein the pseudouridine synthase protein comprises, consists essentially of, or consists of a TRUB1 protein and/or catalytic portion thereof.

Aspect 10 is the composition of aspect 9, wherein the TRUB1 catalytic portion comprises, consists essentially of, or consists of amino acids 1-349, 66-311, 66-280, or 113-280 of the TRUB1 protein.

Aspect 11 is the composition of any one of aspects 1 to 10, wherein the pseudouridine synthase protein consists of or consists essentially of a protein comprising an amino acid sequence at least 80%, 85%, 90%, 95%, 99%, or 100% identical to SEQ ID NO: 6, 8, 10, or 12.

Aspect 12 is the composition of any one of aspects 1 to 11, further comprising one or more guide RNA (gRNA) molecules that target a messenger RNA (mRNA) molecule associated with a target gene.

Aspect 13 is a composition comprising one or more targeting RNA molecules that target a messenger RNA (mRNA) molecule associated with a target gene.

Aspect 14 is the composition of aspect 13, wherein the targeting RNA is a small nucleolar RNA (snoRNA) or a guide RNA (gRNA).

Aspect 15 is the composition of aspect 14, wherein the snoRNA is in vitro transcribed RNA.

Aspect 16 is the composition of any one of aspect 13 to 15, wherein the targeting RNA targets a gene associated with diseases and/or disorders characterized by haploinsufficiency, gene downregulation, and/or gene translation inhibiting polymorphisms.

Aspect 17 is the composition of any one of aspects 13 to 16, wherein the gene translation inhibiting polymorphisms comprise one or more of a premature stop codon, rare codon, and/or missense codon mutation.

Aspect 18 is the composition of any one of aspects 13 to 17, wherein the targeting RNA targets a gene comprising one or more rare codons.

Aspect 19 is the composition of aspect 18, wherein the rare codon is ATA, TTT, CAT, TTA, AAT, or TAT.

Aspect 20 is the composition of aspect 19, wherein the rare codon is ATA.

Aspect 21 is the composition of any one of aspects 18 to 20, wherein the gene comprises more than one rare codon.

Aspect 22 is the composition of any one of aspects 18 to 21, wherein the gene comprises more than one different rare codon.

Aspect 23 is the composition of any one of aspects 13 to 22, wherein the targeting RNA molecule targets a transcript associated with any of genes KRAS, p53, DMD, HBB, IDUA NF1, CDC6, AMFR, SCP2, ERH, CFTR, GLMN, POT1, TBK1, KRIT1, NBN, FANCB, DSC2, LIG4, ATP2C1, PGAP1, RB1, MSH2, RASA1, DSG1, KIF11, CHL1, RAD50, ATP7A, PHIP, SI, APC, ATM, and/or BRCA2.

Aspect 24 is the composition of any one of aspects 13 to 23, wherein the targeting RNA molecule comprises a polynucleotide sequence at least 85%, 90%, 95%, or 100% identical to any one or more of SEQ ID NOs: 21-28.

Aspect 25 is the composition of any one of aspects 13 to 24, wherein the target mRNA is a transcript associated with the p53 gene.

Aspect 26 is the composition of aspect 25, wherein the target mRNA associated with the p53 gene comprises a non-sense mutation.

Aspect 27 is the composition of aspect 26, wherein the non-sense mutation comprises R213X.

Aspect 28 is the composition of any one of aspects 13 to 24, wherein the target mRNA is a transcript associated with the KRAS gene.

Aspect 29 is the composition of any one of aspects 13 to 24, wherein the targeting RNA targets uracil (U) 62, U107, U251, U563, CTA, ATA1, ATA2, ATA3, or a combination thereof of an mRNA associated with the KRAS gene.

Aspect 30 is a composition comprising a pseudouridine synthase protein linked to a targeting protein according to any one of aspects 1 to 24, and a guide RNA according to any one of aspects 13 to 24.

Aspect 31 is a method of modulating translation of a target mRNA comprising, contacting one or more site-specific rare codons in the target mRNA with a) one or more site-specific targeting elements, and/or b) one or more of a pseudouridine synthase protein and/or one or more box H/ACA small nucleolar ribonucleoprotein (H/ACA snoRNP) complex components.

Aspect 32 is the method of aspect 31, wherein b) comprises a pseudouridine synthase protein fused to a targeting protein.

Aspect 33 is the method of aspect 31, wherein b) comprises a DKC1 complex.

Aspect 34 is the method of aspect 31, wherein b) comprises DKC1 isoform 3.

Aspect 35 is the method of aspect 31 or 32, wherein a) comprises a targeting protein and/or polypeptide.

Aspect 36 is the method of any one of aspects 31 to 35, wherein a) comprises an RNA-guided protein.

Aspect 37 is the method of any one of aspects 31 to 36, wherein the targeting element comprises a clustered regularly interspaced short palindromic repeats (CRISPR) Associated (Cas) Protein.

Aspect 38 is the method of aspect 37, wherein the Cas protein endonuclease enzymatic activity is non-functional.

Aspect 39 is the method of aspect 38 or 39, wherein the Cas protein comprises a dCas13d protein.

Aspect 40 is the method of any one of aspects 37 to 39, wherein the Cas protein comprises a sequence identity at least 80%, 85%, 90%, 95%, 99%, or 100% identical to SEQ ID NO: 20.

Aspect 41 is the method of any one of aspects 31 to 40, wherein the pseudouridine synthase protein comprises PUS4 (TRUB1), PUS1, PUS3, PUS7, PUS10, and/or RPUSD2.

Aspect 42 is the method of any one of aspects 31 to 41, wherein the pseudouridine synthase protein comprises a sequence at least 80%, 85%, 90%, 95%, 99%, or 100% identical to one or more of SEQ ID NOs: 6, 8, 10, 12, 2, 4, 14, 16, or 18.

Aspect 43 is the method of any one of aspects 31 to 42, wherein the pseudouridine synthase protein comprises, consists essentially of, or consists of a TRUB1 protein and/or catalytic portion thereof.

Aspect 44 is the composition of aspect 43, wherein the TRUB1 catalytic portion comprises, consists essentially of, or consists of amino acids 1-349, 66-311, 66-280, or 113-280 of the TRUB1 protein.

Aspect 45 is the method of any one of aspects 31 to 44, wherein the pseudouridine synthase protein consists of or consists essentially of a protein comprising a sequence at least 80%, 85%, 90%, 95%, 99%, or 100% identical to SEQ ID NOs: 6, 8, 10, or 12.

Aspect 46 is the method of any one of aspects 31 to 45, wherein the pseudouridine synthase protein consists of or consists essentially of a protein comprising a sequence at least 80%, 85%, 90%, 95%, 99%, or 100% identical to SEQ ID NO: 12.

Aspect 47 is the method of any one of aspects 31 to 46, wherein the targeting element comprises one or more guide RNA (gRNA) molecules.

Aspect 48 is the method of aspect 47, wherein the gRNA targets a gene associated with diseases and/or disorders characterized by haploinsufficiency, gene downregulation, and/or gene translation inhibiting polymorphisms.

Aspect 49 is the method of aspect 48, wherein the gRNA targets a gene comprising one or more rare codons.

Aspect 50 is the method of aspect 49, wherein the gene comprises more than one different rare codon.

Aspect 51 is the method of any one of aspects 31 to 50, wherein the rare codon is ATA, TTT, CAT, TTA, AAT, or TAT.

Aspect 52 is the method of any one of aspects 31 to 51, wherein the rare codon is ATA.

Aspect 53 is the method of any one of aspects 31 to 52, wherein the target mRNA comprises more than one rare codons.

Aspect 54 is the method of any one of aspects 31 to 53, wherein the target mRNA is a transcript associated with any of genes KRAS, p53, DMD, HBB, IDUA NF1, CDC6, AMFR, SCP2, ERH, CFTR, GLMN, POT1, TBK1, KRIT1, NBN, FANCB, DSC2, LIG4, ATP2C1, PGAP1, RB1, MSH2, RASA1, DSG1, KIF11, CHL1, RAD50, ATP7A, PHIP, SI, APC, ATM, and/or BRCA2.

Aspect 55 the method of any one of aspects 31 to 54, wherein the targeting element comprises a polynucleotide sequence at least 85%, 90%, 95%, or 100% identical to any one or more of SEQ ID NOs: 21-28.

Aspect 56 is the composition of any one of aspects 31 to 54, wherein the target mRNA is a transcript associated with the p53 gene.

Aspect 57 is the composition of aspect 56, wherein the target mRNA associated with the p53 gene comprises a non-sense mutation.

Aspect 58 is the composition of aspect 57, wherein the non-sense mutation comprises R213X.

Aspect 59 is the method of any one of aspects 31 to 55, wherein the target mRNA is a transcript associated with the KRAS gene.

Aspect 60 is the composition of any one of aspects 31 to 55, wherein the targeting RNA targets uracil (U) 62, U107, U251, U563, CTA, ATA1, ATA2, ATA3, or a combination thereof of an mRNA associated with the KRAS gene.

Aspect 61 is the method of aspect 59 or 60, wherein the targeting element comprises a polynucleotide sequence at least 85%, 90%, 95%, or 100% identical to any one or more of SEQ ID NOs: 25-28.

Aspect 62 is the method of aspect 61, wherein the targeting element comprises a polynucleotide sequence at least 85%, 90%, 95%, or 100% identical to any one or more of SEQ ID NOs: 27.

Aspect 63 is the method of any one of aspects 59 to 62, wherein targeting the KRAS mRNA increases KRAS protein levels.

Aspect 64 is the method of aspect 31, wherein the targeting element comprises an engineered small nucleolar RNA (snoRNA).

Aspect 65 is the method of aspect 64, wherein the engineered snoRNA is in vitro transcribed.

Aspect 66 is the method of aspect 64 or 65, wherein the engineered snoRNA targets a transcript associated with any of genes KRAS, p53, DMD, HBB, IDUA NF1, CDC6, AMFR, SCP2, ERH, CFTR, GLMN, POT1, TBK1, KRIT1, NBN, FANCB, DSC2, LIG4, ATP2C1, PGAP1, RB1, MSH2, RASA1, DSG1, KIF11, CHL1, RAD50, ATP7A, PHIP, SI, APC, ATM, and/or BRCA2.

Aspect 67 is the method of aspect 64 or 66, wherein the engineered snoRNA targets a transcript associated with KRAS.

Aspect 68 is the method of aspect 67, wherein the engineered sno-RNA comprises, consists essentially of, or consists of a polynucleotide sequence at least 85%, 90%, 95%, or 100% identical to any one or more of SEQ ID NOs: 37-45.

Aspect 69 is the method of aspects 67 or 68, wherein the engineered snoRNA targets uracil (U) 62, U107, U251, U563, CTA, ATA1, ATA2, ATA3, or a combination thereof of an mRNA associated with the KRAS gene.

Aspect 70 is a method of treating a disease and/or disorder in an individual comprising, providing the individual in need thereof with a composition according to any one of aspects 1-30.

Aspect 71 is a method of treating a disease and/or disorder in an individual comprising, performing the method of modulating translation levels of mRNA according to any one of aspects 31 to 68 on the individual in need thereof.

Aspect 72 is the method of aspect 70 or 71, wherein the disease and/or disorder is cancer.

Aspect 73 is the method of any one of aspects 70 to 72, wherein the disease and/or disorder is characterized by haploinsufficiency.

Aspect 74 is a kit comprising a composition of any one of aspects 1 to 30.

Aspect 75 is the use of the composition, method, or kit according to any one of aspects 1 to 74, as a medicament, means for treatment and/or prevention of a disease, means for diagnosis, and/or medical research tool.

In certain aspects, any one or more of the aspects disclosed herein may be specifically excluded from any one or more of another aspect disclosed herein.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or. It is specifically contemplated that A, B, or C may be specifically excluded from an aspect.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

As used herein, the terms “install”, “installing”, or “installation” can refer to modifying an existing nucleotide to an alternate isomer, for example converting uridine to Ψ.

As used herein, the terms “link” or “linked” may be used interchangeably and refer to a conjugation between two or more molecules, e.g., at least two amino acids. A used herein, linked refers to a first nucleic acid sequence covalently joined to a second nucleic acid sequence. The first nucleic acid sequence can be directly joined or juxtaposed to the second nucleic acid sequence or alternatively an intervening sequence can covalently join the first sequence to the second sequence. Linked, as used herein, can also refer to a first amino acid sequence covalently, or non-covalently, joined to a second amino acid sequence. The first amino acid sequence can be directly joined or juxtaposed to the second amino acid sequence or alternatively an intervening sequence can covalently join the first amino acid sequence to the second amino acid sequence. Linked as used herein can also refer to a first amino acid sequence covalently or non-covalently joined to a nucleic acid sequence or a small organic or inorganic molecule. Linked, as used herein, can also refer to a particular peptide, polypeptide, or protein covalently joined to a heterologous protein, polypeptide, or protein sequence with a particular function (e.g., for targeting or localization, as a marker, for enhanced immunogenicity, for purification purposes, etc.).

As used herein, the term “engineered” refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In specific aspects, a polynucleotide or vector is engineered through recombinant nucleic acid technologies, and a cell is engineered through transfection or transduction of an engineered polynucleotide or vector. Cells may be engineered to express heterologous proteins that are not naturally expressed by the cells, either because the heterologous proteins are recombinant or synthetic or because the cells do not naturally express the proteins.

It is specifically contemplated that any limitation discussed with respect to one aspect of the invention may apply to any other aspect of the invention. Furthermore, any composition of the invention may be used in any method of the invention, and any method of the invention may be used to produce or to utilize any composition of the invention. Aspects of an aspect set forth in the Examples are also aspects that may be implemented in the context of aspects discussed elsewhere in a different Example or elsewhere in the application, such as in the Summary, Detailed Description, Claims, and Brief Description of the Drawings.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific aspects presented herein.

FIGS. 1A-1C describes how Ψ sites were found to be more enriched in rare codons, such as ATA, and promoted translation in mouse tissues. FIG. 1A is a box and whisker plot depicting a comparison between enrichment of nonoptimal (e.g., with a relative synonymous codon usage (rscu) of less than 1, orange) and optimal (e.g., with a rscu greater than or equal to 1, blue) T-containing codons in different mouse tissues as analyzed by BID-Seq. For unspecific Ψ assignment in consecutive Uridines in BID-seq, only the enrichment for Ψ sites within codons that didn't have consecutive adjacent Us spanning multiple codons were calculated. The x- and y-axis show the mouse tissues and log 2 codon enrichments, respectively. The enrichments were determined by fisher's exact test. The number values above the plots indicated the P-values between the enrichment of the nonoptimal and the optimal codons as determined by the Wilcoxon rank sum exact test. The results showed that at least the cerebellum, kidney, small intestine, testis, and thymus were significantly enriched in non-optimal codons. FIG. 1B shows the enrichment levels for T-containing codons (x-axis) in different mouse tissues (y-axis) was determined. Non-optimal (rscu<1) codons were indicated by black triangles within the respective circle. The color and size of the circles depicts the enrichment and the median of Ψ modified fractions, respectively. The background color (square) indicated the P-value, with a darker blue shade corresponding to a more significant P-value. The enrichment and P-value were both given by Fisher's exact test. The results showed that at least six rare codons (TTT, CAT, ATA, TTA, AAT, and TAT) were found to be enriched with Ψ in all 12 mouse tissues analyzed. FIG. 1C shows the correlations between the Ψ modification fraction of a specific rare codon, ATA, and the translation efficiency of the corresponding gene in different mouse tissues. The translation efficiency was calculated as the ratio between the protein intensity (PMID: 21183079, which is incorporated herein by reference in its entirety for the purposes described herein) and the gene transcripts per million (TPM). The correlation coefficients and the P-values were given by Pearson's correlation. The results showed that for at least transcripts Snx3, Sucla2, Rps12, and Dhrs4, as the Ψ fraction increased, translation efficiency increased.

FIGS. 2A-2E, describes tethering of Ψ synthetase TRUB1 to dCas13d for programmable Ψ installation in RNA. FIG. 2A is a schematic showing the promotion of protein levels by site-specific introduction of Ψ to rare codons, such as ATA (AUA in RNA), via RNA contact with a guided dCas13d-TRUB1 fusion protein. FIG. 2B depicts the comparisons of Ψ modification fractions in human cells between the control (x-axis) and overexpressed (OE, y-axis) sample pairs for each PUS enzyme, including human cells PUS1 (top left; SEQ ID NO: 2), PUS3 (top middle; SEQ ID NO: 4), PUS4 (TRUB1; top right; SEQ ID NO: 6), PUS7 (bottom left; SEQ ID NO: 14), PUS10 (bottom middle; SEQ ID NO: 16), and RPUSD2 (bottom right, SEQ ID NO: 18). FIGS. 2C-E depict the motif frequency usage sequence and the Ψ modification fraction difference between the control and OE sample pairs identified in FIG. 2B, for PUS1 (FIG. 2C), TRUB1 (FIG. 2D), and PUS7 (FIG. 2E).

FIGS. 3A-3C, show validation of RNA modification using a programmable Ψ installation system. FIG. 3A shows the relative RNA stability difference of ERH, SCP2, AMFR and CDC6 RNA transcripts upon Ψ installation by transgenic guided dCas13d-TRUB1 in endogenous-TRUB1 knockdown (KD) HeLa cells. The results showed that restoration of Ψ installation in ERH, SCP2, AMFR and CDC6 significantly increased mRNA levels relative to transcripts lacking T. FIG. 3B depicts validation of Ψ installation at gRNA-targeted sites as measured by CMC-assisted RT-qPCR assays. The normalized read-through ratios were measured by designed RT-qPCR primers amplifying the region containing the Ψ installation sites. FIG. 3C depicts a scheme for modification of a p53 R213X variant transcript, and Western blot results showing the expression levels of the p53 R213X variant and the full-length protein regulated by guide RNA (“G2”; AAACGTTTCTGTCATCCAAATACTCCA, SEQ ID NO: 35) directed dCas13d-TRUB1, the results showed increased levels of functional full length protein, increased expression of the downstream p21, and decreased levels of R213X protein in test conditions when compared to controls. For FIG. 3A and FIG. 3B, n=3 biologically independent samples; data are presented as mean values±s.d.; NS, P>0.05; *P<0.05; **P<0.01; ***P<0.001; and ****P<0.0001.

FIGS. 4A-4E, show that targeting of a pseudouridine synthase to a rare codon (e.g., ATA, i.e., AUA in RNA) promoted protein expression. FIG. 4A shows Western blots (two repeats, top & bottom) depicting increased KRAS protein levels upon targeting of dCas13d-TRUB1 to a KRAS mRNA guided by a Uridine 251 (U251) targeting gRNA relative to controls. FIG. 4B depicts predicted protein structures for full-length TRUB1 (top left; Met1-Cys349; SEQ ID NO: 6) and truncated TRUB1 variants Leu66-Phe311 (top right; SEQ ID NO: 8), Leu66-Gln280 (bottom left; SEQ ID NO: 10), and Lys113-Gln280 (bottom right; SEQ ID NO: 12). FIG. 4C depicts Western blot results showing increased KRAS protein levels relative to controls, after targeting a truncated version of TRUB1 (e.g., Leu66-Phe311 and/or Lys113-Gln280 variants) linked to a RNA-guided protein (e.g., dCas13d) using a U251 gRNA. FIG. 4D depicts Western blot results showing increased mature full-length CFTR protein derived from F508del mutant transcripts when contacted with an L539 gRNA-guided dCas13d-TRUB1 Lys113-Gln280 variant, or when incubating an L539-targeting snoRNA with DKC1. The results showed increased levels of CFTR protein when utilizing the guided dCas13d-TRUB1 Lys113-Gln280 variant system relative to the snoRNA-DKC1 system. FIG. 4E Shows, without wishing to be bound by theory, a schematic representation of engineered snoRNA-guided endogenous DKC1-mediated targeted Ψ installation in mRNA (top), and Western blot results showing the KRAS protein level differences upon snoRNA targeting of Uridines 61 and 107 (U62 and U107; snoRNA-U62-U107; SEQ ID NO: 37), or Uridines 251 and 563 (U251 and U563; snoRNA-U251-U563; SEQ ID NO: 38) relative to control snoRNA (SEQ ID NO: 36) (bottom).

FIGS. 5A-5B, show that engineered-snoRNAs targeting rare ATA codons promoted protein expression. FIG. 5A depicts Western blot results showing the protein level of the kRAS-Flag reporter gene after delivery of engineered snoRNAs. The engineered-snoRNA targeting the CFTR 1539 and GFP (neg*, CFTR U1616-NULL (SEQ ID NO: 40)) served as an off-target negative control, GAG (A227 (SEQ ID NO: 41)) served as a non-T codon negative control and CTA (CUA in RNA, U68 (SEQ ID NO: 42)) served as a non-rare codon negative control. ATA1 (AUA1 in RNA, U62 (SEQ ID NO: 43)), ATA2 (AUA2 in RNA, U107 (SEQ ID NO: 44)) and ATA3 (AUA3 in RNA, U251 (SEQ ID NO: 45)) were engineered snoRNAs targeting the indicating positions in the kRAS-Flag reporter gene. FIG. 5B shows quantification of results. The expression of kRAS-Flag was normalized to beta-tubulin. Plasmid and RNA indicated cellular expressed and directly delivered engineered-snoRNA, respectively.

### DETAILED DESCRIPTION OF THE INVENTION

Peudouridine (Ψ) is the most abundant RNA modification with an estimated T/U ratio of 7-9%, with most Ψ in ribosome RNA and transfer RNA. The ratio of Ψ/U in mammalian mRNA has also been observed to be about 0.2-0.6%, a frequency comparable to that of m⁶A (PMID: 26075521, PMID: 23177736, each of which are incorporated herein by reference in their entirety for the purposes described herein). Ψ can be catalyzed by stand-alone pseudouridine synthases (PUSs) or a snoRNA guided-RNA complex (snoRNA as the guide RNA recognized by DKC1, dyskerin pseudouridine synthase 1). In the human genome, 13 proteins have been annotated as putative PUS (PMID: 34468991, which is incorporated herein by reference in its entirety for the purposes described herein). The PUSs have been shown to target different RNA species to perform distinct functions, including transcriptome-wide regulation of translation (rRNA and tRNA), splicing, RNA stability, protein-RNA interaction, and response to cellular environments (PMID: 3546171, which is incorporated herein by reference in its entirety for the purposes described herein). The dysregulation or depletion of individual PUSs has been purported to cause defects in RNA metabolism and leads to cellular phenotypes or even disease (PMID: 30526862, PMID: 15108122, PMID: 35121864, each of which are incorporated herein by reference in their entirety for the purposes described herein).

Recently, the Inventors developed bisulfite-induced deletion sequencing (BID-seq) to profile the Ψ transcriptome landscape in single nucleotide resolution. BID-seq revealed abundant Ψ sites in various mouse tissue mRNA (PMID: 36302989, which is incorporated herein by reference in its entirety for the purposes described herein). Methods, compositions, and kits concerning BID-seq can be found in International Patent Application publication WO 2022/232795 A1, filed on Apr. 27, 2022, which is incorporated herein by reference in its entirety. The BID-seq method can facilitate single nucleotide resolution interrogation of a transcriptome, facilitating the assignment of individual Ψ to distinct pseudouridine synthases. Transcriptome features of Ψ suggest that installing Ψ within specific mRNAs may alter protein expression without altering the genome.

While Ψ possesses a similar base-pairing property as U, Ψ can improve base-pairing and base stacking (see e.g., PMID: 24369424, which is incorporated herein by reference in its entirety for the purposes described herein). Complete substitution of uridines with Ψ in an mRNA has been reported to result in higher translation efficiencies, however, these effects (at least partially), could be attributed to a highly structured CDS that extended the half-life of the mRNA (see e.g., PMID: 18797453, which is incorporated herein by reference in its entirety for the purposes described herein). Further, it has been reported that Ψ can promote stop codon readthrough by directly regulating the ribosome decoding process (see e.g., PMID: 27107638, which is incorporated herein by reference in its entirety for the purposes described herein). In some aspects, higher ribosome decoding rates in translation elongation could promote protein expression. In some aspects, as the major isoform of DKC1 is localized in the nucleus, using a native DKC1 protein can result in a relatively low pseudouridine installation efficiency. For example, Song et al. used cytosolic DKC1-isoform 3 to improve pseudouridine installation efficiency. In some aspects, a less noticeable increase in protein expression is observed when utilizing snoRNA and/or DKC1 technologies when compared to a targeting protein/polypeptide based system, such as a dCas13d-TRUB1 system as disclosed herein. Moreover, in some aspects, co-delivery of a targeting protein/polypeptide based system, such as a dCas13d-TRUB1 system, provides an extra layer of pseudouridine installation fine-tuning (e.g., the ability to control and/or regulate the pseudouridine installation system expression and/or activity levels).

Disclosed herein are methods and compositions for modulating gene expression. In some aspects, methods and compositions for modification of RNA species for site directed installation of Ψ are disclosed. In some aspects, site directed installation of Ψ may improve translational efficiency by optimizing the decoding of non-optimal codons. In some aspects, site directed installation of Ψ may improve translational efficiency by optimizing the decoding of non-optimal codons while continuing to encode the same amino acid as the same codon comprising a Uridine. Codon optimality is a well-known cis-regulatory element for translation elongation, which refers to the non-uniform ribosome decoding rate for mRNA codons. Codons with higher decoding rates can be defined as optimal codons (PMID: 29018283, which is incorporated herein by reference in its entirety for the purposes described herein). For amino acids coded by multiple codons, substituting a non-optimal codon with optimal codons often increases translation rate and protein expression. For instance, the F508del mutation has been described as destabilizing the CFTR protein, and codon optimization can alter the translation rate and/or can stabilize the full-length F508del CFTR protein (PMID: 25676312, which is incorporated herein by reference in its entirety for the purposes described herein). Based on the recently obtained Ψ maps in mouse tissue mRNA, created using the novel BID-seq technologies described in (PMID: 36302989, which is incorporated herein by reference in its entirety for the purposes described herein) and International Patent Application publication WO 2022/232795 A1, filed on Apr. 27, 2022, each of which are incorporated herein in their entirety, the inventors asked if site-specific targeting of pseudouridylation enzymes to mRNA could provide a means for altering expression rates (e.g., translation), such as through targeting of rare codons, which may lead to modified translation (and thus protein products) for specific genes (or transcripts derived from the same).

In some aspects, technologies provided herein include distinct advantages when compared to other RNA editing technologies, such as A to I editing. In some aspects, ADAR-based RNA base editors can induce proximal and/or distal off-target edits, which can result in codon alterations and generation of mutations. In some aspects, Ψ editing can be considered benign as it does not change the coding information of mRNA. In addition, in some aspects, A-to-I RNA editing only functions for Adenosine to Inosine conversion, whereas site-specific Ψ installation can be at any Uracil (e.g., U in rare codons, stop codons, etc.).

In certain aspects, technologies provided herein comprising a PUS fusion to a targeting element (e.g., dCas13d-TRUB1) can induce higher levels of gene expression relative to systems that do not comprise a PUS fusion to a targeting element. For example, in some aspects, a PUS fusion to a targeting element protein can improve gene expression relative to a system employing an endogenous pseudouridine synthetaseenzyme (e.g., a snoRNA-DKC1), as the PUS fusion to a targeting element protein is heterologous to a cell, and may not rely on recruiting endogenous proteins. In some aspects, relative to a system that relies upon recruitment of endogenous proteins, a PUS fusion to a targeting element protein can avoid titration of endogenous enzymes away from their natural targets. In some aspects, a truncated PUS, or select domain of a PUS (e.g., a catalytic domain), can be selected for its size and/or catalytic activity rate relative to its corresponding full-length PUS protein. In some aspects, a truncated PUS protein may improve packaging, production, and/or delivery of a truncated PUS fusion to a targeting element relative to a non-truncated PUS fusion to a targeting element. In some aspects, a truncated PUS protein may improve the catalytic rate of a truncated PUS fusion to a targeting element relative to a non-truncated PUS fusion to a targeting element.

In certain aspects, technologies provided herein comprise distinct advantages over other systems described in the art, such as snoRNA-DKC1 systems. For example, snoRNA and DKC1 systems (e.g., comprising DKC1 isoform 3) may rely upon endogenous proteins to conduct mRNA targeting. This may titrate endogenous proteins away from their natural targets, leading to unwanted effects. If a snoRNA and DKC1 system employs transgenic overexpression of DKC1 (e.g., isoform 3), this may lead to unwanted effects, for example, DKC1-isoform 3 has been reported to boost energy metabolism and confer growth advantages, such effects could potentially induce carcinogenesis.

In some aspects, technologies provided herein (e.g., compositions, methods etc., comprising pseudouridine synthase enzymes/complexes and/or pseudouridine installation targeting elements) function broadly in mammalian cells, such as human cells. In some aspects, technologies provided herein can facilitate greater than or equal to about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, pseudouridine installation at user-defined RNA loci in a population of RNA molecules. In some aspects, technologies provided herein can facilitate site-specific pseudouridine installation activity while maintaining low off-target pseudouridine installation activity. In some aspects, technologies provided herein can facilitate site-specific pseudouridine installation with low levels of sequence motif bias.

The current disclosure provides technologies including at least polynucleotides, proteins, polypeptides, and/or vectors, and further provides methods and/or compositions comprising any one or more of the aforementioned components. In certain aspects, polynucleotides may encode sequences comprising pseudouridine synthases, engineered small nucleolar RNA (snoRNA), CRISPR/Cas proteins/polypeptides, and/or ancillary RNA components (e.g., CRISPR RNA (crRNA), trans-activating CRISPR RNA (tracrRNA), single guide RNA (sgRNA), etc.). In certain aspects, one or more of the polynucleotides and/or polypeptides can be engineered.

In certain aspects, polynucleotides, proteins, polypeptides, and/or peptide sequences for wild type or mutant versions of various genes, such as site-specific target genes, have been previously disclosed, and may be found in the recognized computerized databases. In certain aspects, polynucleotides, proteins, polypeptides, and/or peptide sequences for wild type versions of various effector proteins and/or RNA molecules, such as various wild type PUS proteins, snoRNAs, target genes, etc. have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

In certain aspects, provided herein are methods, compositions, polynucleotides, polypeptides, and/or vectors comprising engineered pseudouridine synthase enzymes, engineered Cas proteins, engineered fusion proteins (e.g., pseudouridine synthase enzymes or domains thereof fused or linked to a Cas protein or domain thereof), and/or guide oligonucleotides. In certain aspects, provided herein are methods, compositions, polynucleotides, and/or vectors comprising engineered snoRNA molecules targeting site-specific locations in one or more RNA molecules. In certain aspects, provided herein are methods, compositions, polynucleotides, and/or vectors comprising truncated PUS enzymes linked to a catalytically dead endonuclease and optionally complexed with a guide oligonucleotide.

The oligonucleotides, polypeptides, polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to at least, exactly, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, 750, 775, 800, 825, 850, 875, 900, 925, 950, 1000, 1200, 1266, 1400, 1600, 1800, or 2000 or more contiguous amino acids or nucleic acids, or any range derivable therein, of SEQ ID NOs: 1-39. In specific aspects, the nucleic acid encoding the peptide or polypeptide is codon optimized for expression in a mammal. In certain aspects, the peptide or polypeptide is not naturally occurring and/or is in a combination of peptides or polypeptides.

The polypeptides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, or 422 substitutions (or any range derivable therein). In some aspects, the substitution is with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some aspects, the polypeptide comprises one or more substitutions at one or more amino acid positions selected from amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, or 529 of any of SEQ ID NOs: 2, 4, 6, 8, 10, 12, 14, 16, 18, 20, or 39, wherein each substitution is independently chosen from an amino acid selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine; and wherein the polypeptide is or is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) sequence identity to one of SEQ ID NOs: 2, 4, 6, 8, 10, 12, 14, 16, 18, 20, or 39.

In some aspects, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, or 529 of SEQ TD NOs: 2, 4, 6, 8, 10, 12, 14, 16, 18, 20, or 39.

In some aspects, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, or 529 of SEQ ID NOs: 2, 4, 6, 8, 10, 12, 14, 16, 18, 20, or 39 and have or have at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) sequence identity to one of SEQ ID NOs: 2, 4, 6, 8, 10, 12, 14, 16, 18, 20, or 39.

In some aspects, the protein, polypeptide, or nucleic acid may comprise, comprise at least, or comprise at most 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, 750, 775, 800, 825, 850, 875, 900, 925, 950, 1000, 1200, 1266, 1400, 1600, 1800, or 2000 (or any derivable range therein) contiguous amino acids or nucleic acids of SEQ ID NOs: 1-39.

In some aspects, the polypeptide, protein, or nucleic acid may comprise at least, at most, or exactly 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, 750, 775, 800, 825, 850, 875, 900, 925, 950, 1000, 1200, 1266, 1400, 1600, 1800, or 2000 (or any derivable range therein) contiguous amino acids or nucleic acids of SEQ ID NOs: 1-39 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to one of SEQ ID NOs: 1-39.

In some aspects there is a nucleic acid molecule or polypeptide starting at position 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, or 422, of any of SEQ ID NOs: 1-39 and comprising at least, at most, or exactly 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, 750, 775, 800, 825, 850, 875, 900, 925, 950, 1000, 1200, 1266, 1400, 1600, 1800, or 2000 (or any derivable range therein) contiguous amino acids or nucleic acids of any of SEQ ID NOs: 1-39.

### I. Peudouridine Synthases

Aspects of the present disclosure are directed, at least in part, to methods for modification of RNA species, such as mRNA, via introduction of heterologous pseudouridines. In some aspects, heterologous pseudouridines are installed by a pseudouridine synthase comprising system. In some aspects, a pseudouridine synthase is a pseudouridine synthase that can act on mRNA. In some aspects, a pseudouridine synthase may act on mRNA without installing pseudouridine.

In some aspects, a pseudouridine synthase comprises, consists essentially of, or consists of a human pseudouridine synthase enzyme. In some aspects, a pseudouridine synthase comprises, consists essentially of, or consists of Peudouridine Synthase 1 (PUS1), Peudouridine Synthase 3 (PUS3), Peudouridine Synthase 4 (PUS4; aka TRUB1), Peudouridine Synthase 7 (PUS7), Peudouridine Synthase 10 (PUS10), Dyskerin Peudouridine Synthase 1 (DKC1), and/or Peudouridine Synthase Domain Containing 2 (RPUSD2). In some aspects, a pseudouridine synthase comprises, consists essentially of, or consists of a catalytic domain derived from PUS1, PUS3, PUS4 (TRUB1), PUS7, PUS10, DKC1, and/or RPUSD2.

In some aspects, provided herein are technologies for site-specific pseudouridine installation activity (e.g., pseudouridine in RNA installation activity). In some aspects, enzymatic pseudouridine installation activity generates silent, missense, nonsense, and/or synonymous mutations in target mRNA. In some aspects, enzymatic pseudouridine installation activity generates silent mutations in target mRNA. In some aspects, enzymatic pseudouridine installation activity generates missense mutations in target mRNA. In some aspects, enzymatic pseudouridine installation activity can revert a stop codon in an mRNA into a codon that can be read through by a translational complex.

In certain aspects, the size of a protein or polypeptide (wild-type or modified; including pseudouridine synthases and/or other proteins/polypeptides described herein) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 1000, 1200, 1400, 1600, 1800, or 2000 amino acid residues or nucleic acid residues or greater, and any range derivable therein, or derivative of a corresponding amino sequence. It is contemplated that polypeptides may be mutated by truncation, rendering them shorter than their corresponding wild-type form, also or alternatively, they might be altered by fusing or conjugating a heterologous protein or polypeptide sequence with a particular function (e.g., for targeting or localization, for enhanced immunogenicity, for purification purposes, etc.).

In some aspects, methods of the disclosure comprise incubation of a nucleic acid molecule (e.g., an RNA molecule comprising uridine) under conditions sufficient for generation of higher levels of pseudouridine installation at a site that is conventionally modified by pseudouridine and/or novel (e.g., non-endogenous) pseudouridine installation sites. In some aspects, methods of the disclosure comprise incubation of a nucleic acid molecule (e.g., an RNA molecule comprising uridine) under conditions sufficient for contacting a pseudouridine synthase to a site that is conventionally modified by pseudouridine and/or a novel (e.g., non-endogenous) pseudouridine installation sites. In some aspects, methods of the disclosure comprise incubation of a nucleic acid molecule (e.g., an RNA molecule comprising uridine) under conditions sufficient for contacting a targeting RNA to a site that is conventionally modified by pseudouridine and/or a novel (e.g., non-endogenous) pseudouridine installation sites.

In some aspects, a PUS protein comprises, consists essentially of, or consists of an amino acid sequence or is encoded by a polynucleotide sequence, with at least or equal to, exactly or about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any percentage derivable therein, identity to any one of SEQ ID NOs: 1-18.

SEQ ID NO: 1-Pseudouridine Synthase 1 (PUS1) polynucleotide coding sequence
(SEQ ID NO: 1)
ATGGGCCTCCAGCTTCGCGCGCTGTTGGGAGCCTTCGGACGGTGGACCCTGCGCCTGGGACC
GCGTCCGTCCTGCTCGCCGCGCATGGCCGGGAACGCGGAGCCGCCGCCCGCCGGAGCCGCAT
GCCCCCAGGACCGGAGGTCCTGCAGCGGCCGGGCCGGGGGCGACCGCGTCTGGGAGGACGGA
GAACATCCGGCGAAGAAGCTCAAGAGCGGTGGCGACGAGGAGCGGCGCGAGAAGCCGCCCAA
GCGGAAGATCGTGCTGCTCATGGCCTATTCGGGCAAGGGCTACCACGGCATGCAGAGGAATG
TCGGGTCCTCACAATTCAAAACAATTGAAGATGACTTGGTGTCCGCCCTCGTCCGGTCAGGC
TGTATTCCTGAAAATCATGGTGAGGACATGAGGAAAATGTCCTTCCAGCGCTGCGCCCGGAC
AGACAAGGGTGTGTCCGCAGCCGGCCAGGTGGTATCCCTGAAGGTGTGGCTGATTGACGACA
TTCTAGAAAAGATCAACAGCCACCTTCCCTCTCACATTCGGATTCTGGGACTGAAGCGGGTC
ACGGGCGGGTTTAACTCCAAGAACAGATGTGATGCCAGGACCTATTGCTACCTGCTGCCCAC
GTTTGCCTTTGCGCACAAGGACCGGGACGTTCAGGATGAGACCTACCGCCTGAGCGCCGAGA
CGCTGCAGCAGGTCAACAGGCTCCTGGCCTGCTACAAGGGCACGCACAACTTCCACAATTTC
ACCTCGCAGAAGGGGCCGCAGGATCCCAGTGCCTGCCGCTACATCCTGGAGATGTACTGCGA
GGAACCCTTTGTGCGGGAGGGCCTGGAGTTTGCGGTGATCAGGGTGAAGGGCCAGAGCTTCA
TGATGCATCAGATCCGGAAGATGGTCGGCCTGGTGGTGGCCATTGTGAAGGGTTATGCCCCT
GAGAGCGTGCTGGAGCGCAGCTGGGGCACAGAGAAGGTGGACGTGCCCAAGGCGCCCGGACT
CGGCCTGGTCCTGGAGAGGGTGCACTTCGAGAAGTACAACCAGCGCTTTGGCAACGATGGGC
TGCATGAGCCGCTGGACTGGGCGCAGGAGGAAGGAAAGGTCGCAGCCTTCAAGGAGGAGCAC
ATCTACCCCACCATCATCGGCACCGAGCGGGACGAACGCTCCATGGCCCAGTGGCTGAGCAC
CTTGCCCATCCACAACTTCAGTGCCACCGCTCTCACGGCAGGTGGCACGGGCGCCAAGGTGC
CCAGTCCCCTGGAAGGCAGTGAAGGGGACGGAGACACTGAC
SEQ ID NO: 2-Pseudouridine Synthase 1 (PUS1) amino acid sequence
(SEQ ID NO: 2)
MGLQLRALLGAFGRWTLRLGPRPSCSPRMAGNAEPPPAGAACPQDRRSCSGRAGGDRVWEDG
EHPAKKLKSGGDEERREKPPKRKIVLLMAYSGKGYHGMQRNVGSSQFKTIEDDLVSALVRSG
CIPENHGEDMRKMSFQRCARTDKGVSAAGQVVSLKVWLIDDILEKINSHLPSHIRILGLKRV
TGGFNSKNRCDARTYCYLLPTFAFAHKDRDVQDETYRLSAETLQQVNRLLACYKGTHNFHNF
TSQKGPQDPSACRYILEMYCEEPFVREGLEFAVIRVKGQSFMMHQIRKMVGLVVAIVKGYAP
ESVLERSWGTEKVDVPKAPGLGLVLERVHFEKYNQRFGNDGLHEPLDWAQEEGKVAAFKEEH
IYPTIIGTERDERSMAQWLSTLPIHNFSATALTAGGTGAKVPSPLEGSEGDGDTD
SEQ ID NO: 3-Pseudouridine Synthase 3 (PUS3) polynucleotide coding sequence
(SEQ ID NO: 3)
ATGGCTGATAATGACACAGACAGAAACCAGACTGAGAAGCTCCTAAAAAGAGTACGAGAACT
GGAGCAAGAGGTGCAAAGACTTAAAAAGGAACAGGCCAAAAATAAGGAGGACTCAAACATTA
GAGAAAATTCATCAGGAGCTGGAAAAACTAAGCGTGCATTTGATTTCAGTGCTCATGGCCGA
AGACACGTAGCCCTAAGAATAGCCTATATGGGCTGGGGATACCAGGGCTTTGCTAGTCAGGA
AAACACAAATAATACCATTGAAGAGAAACTGTTTGAAGCTCTAACCAAGACTCGACTAGTAG
AAAGCAGACAGACATCCAACTATCACCGATGTGGGAGAACAGATAAAGGAGTTAGTGCCTTT
GGACAGGTGATCTCACTTGACCTTCGCTCTCAGTTTCCAAGGGGCAGGGATTCCGAGGACTT
TAATGTAAAAGAGGAGGCTAATGCTGCTGCTGAAGAGATCCGTTATACCCACATTCTCAATC
GGGTACTCCCTCCAGACATCCGTATATTGGCCTGGGCCCCTGTAGAACCAAGCTTCAGTGCT
AGGTTCAGCTGCCTTGAGCGGACTTACCGCTATTTTTTCCCTCGTGCTGATTTAGATATTGT
AACCATGGATTATGCAGCTCAGAAGTATGTTGGCACCCATGATTTCAGGAACTTGTGTAAAA
TGGATGTAGCCAACGGTGTGATTAATTTTCAGAGGACTATTCTATCTGCTCAAGTACAGCTA
GTGGGCCAGAGCCCAGGTGAGGGGAGATGGCAAGAACCTTTCCAGTTATGTCAGTTTGAAGT
GACTGGCCAGGCATTCCTTTATCATCAAGTCCGATGTATGATGGCTATCCTCTTTCTGATTG
GCCAAGGAATGGAGAAGCCAGAGATTATTGATGAGCTGCTGAATATAGAGAAAAATCCCCAA
AAGCCTCAATATAGTATGGCTGTAGAATTTCCTCTAGTCTTATATGACTGTAAGTTTGAAAA
TGTCAAGTGGATCTATGACCAGGAGGCTCAGGAGTTCAATATTACCCACCTACAACAACTGT
GGGCTAATCATGCTGTCAAAACTCACATGTTGTATAGTATGCTACAAGGACTGGACACTGTT
CCAGTACCCTGTGGAATAGGACCAAAGATGGATGGAATGACAGAATGGGGAAATGTTAAGCC
CTCTGTCATAAAGCAGACCAGTGCCTTTGTAGAAGGAGTGAAGATGCGCACATATAAGCCCC
TCATGGACCGTCCTAAATGCCAAGGACTGGAATCCCGGATCCAGCATTTTGTACGTAGGGGA
CGAATTGAGCACCCACATTTATTCCATGAGGAAGAAACAAAAGCCAAAAGGGACTGTAATGA
CACACTAGAGGAAGACAATACTAATTTGGAGACACCAACGAAGAGGGTCTGTGTTGACACAG
AAATTAAAAGCATCATT
SEQ ID NO: 4-Pseudouridine Synthase 3 (PUS3) amino acid sequence
(SEQ ID NO: 4)
MADNDTDRNQTEKLLKRVRELEQEVQRLKKEQAKNKEDSNIRENSSGAGKTKRAFDFSAHGR
RHVALRIAYMGWGYQGFASQENTNNTIEEKLFEALTKTRLVESRQTSNYHRCGRTDKGVSAF
GQVISLDLRSQFPRGRDSEDENVKEEANAAAEEIRYTHILNRVLPPDIRILAWAPVEPSFSA
RFSCLERTYRYFFPRADLDIVTMDYAAQKYVGTHDFRNLCKMDVANGVINFQRTILSAQVQL
VGQSPGEGRWQEPFQLCQFEVTGQAFLYHQVRCMMAILFLIGQGMEKPEIIDELLNIEKNPQ
KPQYSMAVEFPLVLYDCKFENVKWIYDQEAQEFNITHLQQLWANHAVKTHMLYSMLQGLDTV
PVPCGIGPKMDGMTEWGNVKPSVIKQTSAFVEGVKMRTYKPLMDRPKCQGLESRIQHFVRRG
RIEHPHLFHEEETKAKRDCNDTLEEDNTNLETPTKRVCVDTEIKSII
SEQ ID NO: 5-Pseudouridine Synthase 4 (PUS4; aka TRUB1) polynucleotide coding
sequence
(SEQ ID NO: 5)
ATGGCCGCTTCTGAGGCGGCGGTGGTGTCTTCGCCGTCTTTGAAAACAGACACATCCCCTGT
CCTTGAAACTGCAGGAACGGTCGCAGCAATGGCTGCGACCCCGTCAGCAAGGGCTGCAGCCG
CGGTGGTTGCGGCCGCGGCCAGGACCGGATCCGAAGCCAGGGTCTCCAAGGCCGCTTTGGCT
ACCAAGCTGCTGTCCTTGAGCGGCGTGTTCGCCGTGCACAAGCCCAAAGGGCCCACTTCAGC
CGAGCTGCTGAATCGGTTGAAGGAGAAGCTGCTGGCAGAAGCTGGAATGCCTTCTCCAGAAT
GGACCAAGAGGAAAAAGCAGACTTTGAAAATTGGGCATGGAGGGACTCTAGACAGCGCAGCC
CGAGGAGTTCTGGTTGTTGGAATTGGAAGCGGAACAAAAATGTTGACCAGTATGTTGTCAGG
GTCCAAGAGATATACTGCCATTGGAGAACTGGGGAAAGCTACTGATACACTAGATTCTACGG
GGAGGGTAACAGAAGAAAAACCTTACGATAAAATAACACAAGAAGATATTGAAGGCATTCTA
CAGAAATTTACTGGAAATATAATGCAAGTGCCCCCCCTCTATTCTGCATTAAAGAAAGATGG
ACAAAGACTTTCGACTTTGATGAAGAGAGGTGAAGTCGTAGAAGCAAAACCTGCCAGGCCAG
TGACTGTATACAGTATCTCCCTTCAAAAATTCCAGCCACCATTTTTCACATTAGATGTTGAA
TGTGGAGGAGGTTTTTATATCAGAAGCTTGGTCAGTGACATTGGAAAAGAACTATCTTCCTG
TGCCAATGTGCTAGAGCTGACCCGAACCAAACAGGGACCATTTACGCTAGAAGAACATGCCC
TTCCTGAAGACAAATGGACAATTGATGACATTGCACAGTCTCTTGAGCATTGCTCATCTCTT
TTCCCAGCAGAGTTGGCACTTAAAAAATCAAAACCTGAGTCTAATGAACAGGTTTTGAGCTG
TGAATATATAACTCTAAATGAGCCAAAGAGAGAAGATGATGTAATTAAGACGTGT
SEQ ID NO: 6-Pseudouridine Synthase 4 (PUS4; aka TRUB1) amino acid sequence
(SEQ ID NO: 6)
MAASEAAVVSSPSLKTDTSPVLETAGTVAAMAATPSARAAAAVVAAAARTGSEARVSKAALA
TKLLSLSGVFAVHKPKGPTSAELLNRLKEKLLAEAGMPSPEWTKRKKQTLKIGHGGTLDSAA
RGVLVVGIGSGTKMLTSMLSGSKRYTAIGELGKATDTLDSTGRVTEEKPYDKITQEDIEGIL
QKFTGNIMQVPPLYSALKKDGQRLSTLMKRGEVVEAKPARPVTVYSISLQKFQPPFFTLDVE
CGGGFYIRSLVSDIGKELSSCANVLELTRTKQGPFTLEEHALPEDKWTIDDIAQSLEHCSSL
FPAELALKKSKPESNEQVLSCEYITLNEPKREDDVIKTC
SEQ ID NO: 7-Truncated TRUB1 (Leu66-Phe311) polynucleotide coding sequence
(SEQ ID NO: 7)
ATGCTGTCCTTGAGCGGCGTGTTCGCCGTGCACAAGCCCAAAGGGCCCACTTCAGCCGAGCT
GCTGAATCGGTTGAAGGAGAAGCTGCTGGCAGAAGCTGGAATGCCTTCTCCAGAATGGACCA
AGAGGAAAAAGCAGACTTTGAAAATTGGGCATGGAGGGACTCTAGACAGCGCAGCCCGAGGA
GTTCTGGTTGTTGGAATTGGAAGCGGAACAAAAATGTTGACCAGTATGTTGTCAGGGTCCAA
GAGATATACTGCCATTGGAGAACTGGGGAAAGCTACTGATACACTAGATTCTACGGGGAGGG
TAACAGAAGAAAAACCTTACGATAAAATAACACAAGAAGATATTGAAGGCATTCTACAGAAA
TTTACTGGAAATATAATGCAAGTGCCCCCCCTCTATTCTGCATTAAAGAAAGATGGACAAAG
ACTTTCGACTTTGATGAAGAGAGGTGAAGTCGTAGAAGCAAAACCTGCCAGGCCAGTGACTG
TATACAGTATCTCCCTTCAAAAATTCCAGCCACCATTTTTCACATTAGATGTTGAATGTGGA
GGAGGTTTTTATATCAGAAGCTTGGTCAGTGACATTGGAAAAGAACTATCTTCCTGTGCCAA
TGTGCTAGAGCTGACCCGAACCAAACAGGGACCATTTACGCTAGAAGAACATGCCCTTCCTG
AAGACAAATGGACAATTGATGACATTGCACAGTCTCTTGAGCATTGCTCATCTCTTTTC
SEQ ID NO: 8-Truncated TRUB1 (Leu66-Phe311) amino acid sequence
(SEQ ID NO: 8)
MLSLSGVFAVHKPKGPTSAELLNRLKEKLLAEAGMPSPEWTKRKKQTLKIGHGGTLDSAARG
VLVVGIGSGTKMLTSMLSGSKRYTAIGELGKATDTLDSTGRVTEEKPYDKITQEDIEGILQK
FTGNIMQVPPLYSALKKDGQRLSTLMKRGEVVEAKPARPVTVYSISLQKFQPPFFTLDVECG
GGFYIRSLVSDIGKELSSCANVLELTRTKQGPFTLEEHALPEDKWTIDDIAQSLEHCSSLF
SEQ ID NO: 9-Truncated TRUB1 (Leu66-Gln280) polynucleotide coding sequence
(SEQ ID NO: 9)
ATGCTGTCCTTGAGCGGCGTGTTCGCCGTGCACAAGCCCAAAGGGCCCACTTCAGCCGAGCT
GCTGAATCGGTTGAAGGAGAAGCTGCTGGCAGAAGCTGGAATGCCTTCTCCAGAATGGACCA
AGAGGAAAAAGCAGACTTTGAAAATTGGGCATGGAGGGACTCTAGACAGCGCAGCCCGAGGA
GTTCTGGTTGTTGGAATTGGAAGCGGAACAAAAATGTTGACCAGTATGTTGTCAGGGTCCAA
GAGATATACTGCCATTGGAGAACTGGGGAAAGCTACTGATACACTAGATTCTACGGGGAGGG
TAACAGAAGAAAAACCTTACGATAAAATAACACAAGAAGATATTGAAGGCATTCTACAGAAA
TTTACTGGAAATATAATGCAAGTGCCCCCCCTCTATTCTGCATTAAAGAAAGATGGACAAAG
ACTTTCGACTTTGATGAAGAGAGGTGAAGTCGTAGAAGCAAAACCTGCCAGGCCAGTGACTG
TATACAGTATCTCCCTTCAAAAATTCCAGCCACCATTTTTCACATTAGATGTTGAATGTGGA
GGAGGTTTTTATATCAGAAGCTTGGTCAGTGACATTGGAAAAGAACTATCTTCCTGTGCCAA
TGTGCTAGAGCTGACCCGAACCAAACAG
SEQ ID NO: 10-Truncated TRUB1 (Leu66-Gln280) amino acid sequence
(SEQ ID NO: 10)
MLSLSGVFAVHKPKGPTSAELLNRLKEKLLAEAGMPSPEWTKRKKQTLKIGHGGTLDSAARG
VLVVGIGSGTKMLTSMLSGSKRYTAIGELGKATDTLDSTGRVTEEKPYDKITQEDIEGILQK
FTGNIMQVPPLYSALKKDGQRLSTLMKRGEVVEAKPARPVTVYSISLQKFQPPFFTLDVECG
GGFYIRSLVSDIGKELSSCANVLELTRTKQ
SEQ ID NO: 11-Truncated TRUB1 (Lys113-Gln280) polynucleotide coding sequence
(SEQ ID NO: 11)
ATGAAAATTGGGCATGGAGGGACTCTAGACAGCGCAGCCCGAGGAGTTCTGGTTGTTGGAAT
TGGAAGCGGAACAAAAATGTTGACCAGTATGTTGTCAGGGTCCAAGAGATATACTGCCATTG
GAGAACTGGGGAAAGCTACTGATACACTAGATTCTACGGGGAGGGTAACAGAAGAAAAACCT
TACGATAAAATAACACAAGAAGATATTGAAGGCATTCTACAGAAATTTACTGGAAATATAAT
GCAAGTGCCCCCCCTCTATTCTGCATTAAAGAAAGATGGACAAAGACTTTCGACTTTGATGA
AGAGAGGTGAAGTCGTAGAAGCAAAACCTGCCAGGCCAGTGACTGTATACAGTATCTCCCTT
CAAAAATTCCAGCCACCATTTTTCACATTAGATGTTGAATGTGGAGGAGGTTTTTATATCAG
AAGCTTGGTCAGTGACATTGGAAAAGAACTATCTTCCTGTGCCAATGTGCTAGAGCTGACCC
GAACCAAACAG
SEQ ID NO: 12-Truncated TRUB1 (Lys113-Gln280) amino acid sequence
(SEQ ID NO: 12)
MKIGHGGTLDSAARGVLVVGIGSGTKMLTSMLSGSKRYTAIGELGKATDTLDSTGRVTEEKP
YDKITQEDIEGILQKFTGNIMQVPPLYSALKKDGQRLSTLMKRGEVVEAKPARPVTVYSISL
QKFQPPFFTLDVECGGGFYIRSLVSDIGKELSSCANVLELTRTKQ
SEQ ID NO: 13-Pseudouridine Synthase 7 (PUS7) polynucleotide coding sequence
(SEQ ID NO: 13)
ATGGAGATGACAGAAATGACTGGTGTGTCGCTGAAACGTGGGGCACTGGTTGTCGAAGATAA
TGACAGTGGAGTCCCAGTTGAAGAGACAAAAAAACAGAAGCTGTCGGAATGCAGCCTAACCA
AAGGTCAAGATGGGCTACAGAATGACTTTCTGTCCATCAGTGAAGACGTGCCTCGGCCTCCT
GACACTGTCAGTACTGGGAAAGGTGGAAAGAATTCTGAGGCTCAGTTGGAAGATGAGGAAGA
AGAGGAGGAAGATGGACTTTCAGAGGAGTGCGAGGAGGAGGAATCAGAGAGTTTTGCAGACA
TGATGAAGCATGGACTCACTGAGGCTGACGTAGGCATCACCAAGTTTGTGAGTTCTCATCAA
GGGTTCTCGGGAATCTTAAAAGAAAGATACTCCGACTTCGTTGTTCATGAAATAGGAAAAGA
TGGACGGATCAGCCATTTGAATGACTTGTCCATTCCAGTGGATGAGGAGGACCCTTCAGAAG
ACATATTTACAGTTTTGACAGCTGAAGAAAAGCAGCGATTGGAAGAGCTCCAGCTGTTCAAA
AATAAGGAAACCAGTGTTGCCATTGAGGTTATCGAGGACACCAAAGAGAAAAGAACCATCAT
CCATCAGGCTATCAAATCTCTGTTTCCAGGATTAGAGACAAAAACAGAGGATAGGGAGGGGA
AGAAATACATTGTAGCCTACCACGCAGCTGGGAAAAAGGCTTTGGCAAATCCAAGAAAACAT
TCTTGGCCAAAATCTAGGGGAAGTTACTGCCACTTCGTACTATATAAGGAAAACAAAGACAC
CATGGATGCTATTAATGTACTCTCCAAATACTTAAGAGTCAAGCCAAATATATTCTCCTACA
TGGGAACCAAAGATAAAAGGGCTATAACAGTTCAAGAAATTGCTGTTCTCAAAATAACTGCA
CAAAGACTTGCCCACCTGAATAAGTGCTTGATGAACTTTAAGCTAGGGAATTTCAGCTATCA
AAAAAACCCACTGAAATTGGGAGAGCTTCAAGGAAACCACTTCACTGTTGTTCTCAGAAATA
TAACAGGAACTGATGACCAAGTACAGCAAGCTATGAACTCTCTCAAGGAGATTGGATTTATT
AACTACTATGGAATGCAAAGATTTGGAACCACAGCTGTCCCTACGTATCAGGTTGGAAGAGC
TATACTACAAAATTCCTGGACAGAAGTCATGGATTTAATATTGAAACCCCGCTCTGGAGCTG
AAAAGGGCTACTTGGTTAAATGCAGAGAAGAATGGGCAAAGACCAAAGACCCAACTGCTGCC
CTCAGAAAACTACCTGTCAAAAGGTGTGTGGAAGGGCAGCTGCTTCGAGGACTTTCAAAATA
TGGAATGAAGAATATAGTCTCTGCATTTGGCATAATACCCAGAAATAATCGCTTAATGTATA
TTCATAGCTACCAAAGCTATGTGTGGAATAACATGGTAAGCAAGAGGATAGAAGACTATGGA
CTAAAACCTGTTCCAGGGGACCTCGTTCTCAAAGGAGCCACAGCCACCTATATTGAGGAAGA
TGATGTTAATAATTACTCTATCCATGATGTGGTAATGCCCTTGCCTGGTTTCGATGTTATCT
ACCCAAAGCATAAAATTCAAGAAGCCTACAGGGAAATGCTCACAGCTGACAATCTTGATATT
GACAACATGAGACACAAAATTCGAGATTATTCCTTGTCAGGGGCCTACCGAAAGATCATTAT
TCGTCCTCAGAATGTTAGCTGGGAAGTCGTTGCATATGATGATCCCAAAATTCCACTTTTCA
ACACAGATGTGGACAACCTAGAAGGGAAGACACCACCAGTTTTTGCTTCTGAAGGCAAATAC
AGGGCTCTGAAAATGGATTTTTCTCTACCCCCTTCTACTTACGCCACCATGGCCATTCGAGA
AGTGCTAAAAATGGATACCAGTATCAAGAACCAGACGCAGCTGAATACAACCTGGCTTCGC
SEQ ID NO: 14-Pseudouridine Synthase 7 (PUS7) amino acid sequence
(SEQ ID NO: 14)
MEMTEMTGVSLKRGALVVEDNDSGVPVEETKKQKLSECSLTKGQDGLQNDFLSISEDVPRPP
DTVSTGKGGKNSEAQLEDEEEEEEDGLSEECEEEESESFADMMKHGLTEADVGITKFVSSHQ
GFSGILKERYSDFVVHEIGKDGRISHLNDLSIPVDEEDPSEDIFTVLTAEEKQRLEELQLFK
NKETSVAIEVIEDTKEKRTIIHQAIKSLFPGLETKTEDREGKKYIVAYHAAGKKALANPRKH
SWPKSRGSYCHFVLYKENKDTMDAINVLSKYLRVKPNIFSYMGTKDKRAITVQEIAVLKITA
QRLAHLNKCLMNFKLGNFSYQKNPLKLGELQGNHFTVVLRNITGTDDQVQQAMNSLKEIGFI
NYYGMQRFGTTAVPTYQVGRAILQNSWTEVMDLILKPRSGAEKGYLVKCREEWAKTKDPTAA
LRKLPVKRCVEGQLLRGLSKYGMKNIVSAFGIIPRNNRLMYIHSYQSYVWNNMVSKRIEDYG
LKPVPGDLVLKGATATYIEEDDVNNYSIHDVVMPLPGFDVIYPKHKIQEAYREMLTADNLDI
DNMRHKIRDYSLSGAYRKIIIRPQNVSWEVVAYDDPKIPLENTDVDNLEGKTPPVFASEGKY
RALKMDESLPPSTYATMAIREVLKMDTSIKNQTQLNTTWLR
SEQ ID NO: 15-Pseudouridine Synthase 10 (PUS10) polynucleotide coding sequence
(SEQ ID NO: 15)
ATGTTCCCACTGACTGAGGAAAACAAGCATGTGGCCCAGTTGTTGCTCAATACTGGTACTTG
TCCAAGATGTATCTTCAGATTCTGTGGTGTGGATTTTCATGCACCTTACAAACTTCCATACA
AGGAGTTGCTCAATGAACTACAGAAATTTCTGGAAACTGAAAAAGATGAATTAATTTTGGAA
GTTATGAACCCACCTCCCAAGAAAATTCGACTGCAAGAACTGGAAGATAGTATTGATAATCT
AAGTCAAAATGGAGAGGGAAGGATCTCTGTTAGTCATGTTGGAAGCACTGCTTCCAAGAACT
CAAATTTAAATGTATGTAATGTATGCCTAGGAATTCTTCAAGAATTCTGTGAGAAAGATTTC
ATTAAAAAGGTGTGCCAAAAGGTTGAGGCCTCTGGGTTTGAATTCACCAGCTTGGTATTTTC
AGTCTCCTTCCCACCACAACTATCTGTAAGAGAGCATGCTGCATGGTTGCTGGTAAAACAGG
AAATGGGAAAGCAGAGTCTGTCGCTGGGAAGAGATGATATAGTTCAGCTAAAAGAAGCCTAC
AAATGGATAACTCACCCCCTGTTTTCAGAGGAACTGGGTGTTCCCATTGATGGAAAGAGCTT
GTTTGAAGTGAGTGTGGTCTTTGCTCACCCAGAAACAGTTGAGGATTGCCACTTCCTAGCTG
CGATATGCCCAGATTGTTTTAAGCCAGCCAAAAACAAACAGTCTGTATTCACTAGAATGGCA
GTTATGAAAGCCTTGAATAAGATAAAGGAAGAGGATTTCCTTAAGCAGTTTCCTTGTCCTCC
AAACTCACCAAAGGCTGTATGCGCTGTTCTTGAAATTGAATGTGCTCATGGTGCTGTTTTTG
TGGCTGGGAGATATAATAAATACTCCAGGAATCTACCACAAACTCCTTGGATAATTGATGGA
GAAAGGAAGCTGGAATCTTCAGTGGAAGAATTAATTTCAGATCATCTGTTGGCAGTATTTAA
AGCAGAGAGTTTTAATTTTTCATCCTCTGGAAGAGAAGATGTAGATGTGAGAACATTAGGAA
ATGGAAGGCCCTTTGCAATTGAGCTGGTGAATCCTCATAGAGTACATTTCACTTCACAAGAA
ATTAAGGAACTTCAGCAGAAAATTAATAACTCATCTAACAAAATCCAAGTACGTGACTTGCA
GCTTGTCACAAGAGAGGCAATAGGACATATGAAAGAAGGTGAAGAAGAAAAGACAAAGACCT
ACAGTGCCTTAATTTGGACAAATAAAGCGATACAGAAGAAAGACATTGAATTCCTAAATGAC
ATAAAGGACTTAAAAATCGACCAGAAAACACCTTTGCGCGTCCTTCACCGAAGGCCCCTGGC
TGTGCGAGCTCGCGTCATTCACTTCATGGAGACACAGTACGTGGATGAGCACCACTTCCGCC
TCCACTTGAAAACTCAGGCTGGCACCTACATTAAAGAGTTTGTACATGGAGACTTCGGGAGA
ACCAAGCCAAACATTGGGTCCCTGATGAATGTGACTGCAGACATTCTGGAGCTGGATGTTGA
GTCTGTAGATGTTGACTGGCCACCTGCTCTGGATGAC
SEQ ID NO: 16-Pseudouridine Synthase 10 (PUS10) amino acid sequence
(SEQ ID NO: 16)
MFPLTEENKHVAQLLLNTGTCPRCIFRFCGVDFHAPYKLPYKELLNELQKFLETEKDELILE
VMNPPPKKIRLQELEDSIDNLSQNGEGRISVSHVGSTASKNSNLNVCNVCLGILQEFCEKDE
IKKVCQKVEASGFEFTSLVFSVSFPPQLSVREHAAWLLVKQEMGKQSLSLGRDDIVQLKEAY
KWITHPLFSEELGVPIDGKSLFEVSVVFAHPETVEDCHFLAAICPDCFKPAKNKQSVFTRMA
VMKALNKIKEEDFLKQFPCPPNSPKAVCAVLEIECAHGAVEVAGRYNKYSRNLPQTPWIIDG
ERKLESSVEELISDHLLAVFKAESFNFSSSGREDVDVRTLGNGRPFAIELVNPHRVHFTSQE
IKELQQKINNSSNKIQVRDLQLVTREAIGHMKEGEEEKTKTYSALIWTNKAIQKKDIEFLND
IKDLKIDQKTPLRVLHRRPLAVRARVIHFMETQYVDEHHFRLHLKTQAGTYIKEFVHGDEGR
TKPNIGSLMNVTADILELDVESVDVDWPPALDD
SEQ ID NO: 17-RNA Pseudouridine Synthase Domain Containing 2 (RPUSD2)
polynucleotide coding sequence
(SEQ ID NO: 17)
ATGTGGCTGGACCGCCGCGGATGGCTCAGGGTTCTTGGACATTGGCGCTACGACCTTAGGCG
CCCTAGCTTTACCAGGACTTGGAGTGGCGATAAGGGCCCAATGGCAGAAACAGTGTCTACCC
AGGTTGGGACAGAGGGCGGGCTGAGGGCTTCGCATCAGCAAAACGGTGACGCTGGTGGCGAC
GCGAAGGTTGAGCTGTCCCCCGGGCCCCCGAAGCCGGCTGGCCGGGAAGTGGAGCCGGCCCC
AGTAGGCGGGGAGCATCCCTCGGCTGCAGCCCCAGGCCCGGGCAAGCATAAGAAGCGGCGGG
GCGCAACCAGGGAGCGTGTCGTGCCGCCCCCGAAGAAGCGGCGGACCGGGGTGAGCTTCGGA
GATGAGCACTTTGCAGAAACCAGTTATTACTTCGAGGGCGGCCTGCGTAAGGTGCGGCCCTA
TTACTTTGACTTCCGGACCTACTGCAAAGGTCGCTGGGGGGCCACAGCTTGCTGCACGTCT
TCAGTACCGAGTTCCGAGCTCAGCCCCTGGCCTACTATGAGGCCGCGGTCCGGGCGGGCCGC
CTGCAACTCAACGAGAAGCCGGTGCAGGACCTCAACATCGTGCTCAAGGACAATGATTTCTT
GCGGAACACAGTGCACAGGCATGAGCCACCAGTCACAGCAGAGCCCATTCGCCTGCTAGCTG
AGAACGAAGATGTGGTGGTTGTAGACAAGCCTTCCTCCATTCCCGTTCACCCCTGTGGCCGC
TTCCGACACAACACAGTTATCTTCATCCTAGGCAAGGAGCACCAACTCAAGGAGCTACACCC
CTTGCATCGGCTTGACCGCCTTACCTCAGGGGTGCTTATGTTTGCCAAGACAGCTGCAGTCT
CTGAGAGAATTCACGAGCAGGTTCGGGACCGGCAGCTGGAGAAGGAGTACGTGTGCCGGGTG
GAAGGGGAGTTCCCCACTGAGGAAGTGACCTGTAAAGAACCCATCTTAGTGGTGTCTTACAA
AGTAGGGGTGTGCCGTGTAGATCCCCGGGGCAAGCCCTGTGAGACAGTGTTCCAGAGGCTAA
GCTACAATGGCCAGTCCAGTGTGGTACGGTGCCGGCCACTCACAGGCCGCACACACCAGATT
CGAGTCCACCTTCAGTTCTTGGGCCATCCCATTCTCAACGACCCCATCTACAACTCAGTTGC
CTGGGGTCCTTCTCGAGGCCGGGGCGGCTACATTCCCAAGACAAACGAGGAGTTGCTACGGG
ACCTGGTAGCAGAGCACCAGGCCAAACAGAGCCTGGATGTGCTAGATCTCTGTGAGGGTGAC
CTGTCCCCAGGACTCACAGACTCTACGGCCCCCTCCTCAGAGTTGGGCAAGGACGACCTGGA
AGAGTTGGCTGCAGCTGCCCAGAAGATGGAGGAAGTAGCTGAGGCAGCCCCTCAGGAGTTGG
ACACAATAGCCTTGGCATCAGAGAAGGCAGTTGAAACAGATGTCATGAATCAAGAGACAGAC
CCACTCTGTGCAGAGTGCCGGCTGGTGCGACAGGATCCCTTGCCCCAAGACCTTGTGATGTT
CCTACATGCCCTACGCTATAAAGGGCCAGGCTTTGAGTACTTTTCACCAATGCCTGCCTGGG
CACAGGATGACTGGCAAAAGGAC
SEQ ID NO: 18-RNA Pseudouridine Synthase Domain Containing 2 (RPUSD2) amino acid
sequence
(SEQ ID NO: 18)
MWLDRRGWLRVLGHWRYDLRRPSFTRTWSGDKGPMAETVSTQVGTEGGLRASHQQNGDAGGD
AKVELSPGPPKPAGREVEPAPVGGEHPSAAAPGPGKHKKRRGATRERVVPPPKKRRTGVSFG
DEHFAETSYYFEGGLRKVRPYYFDFRTYCKGRWVGHSLLHVESTEFRAQPLAYYEAAVRAGR
LQLNEKPVQDLNIVLKDNDFLRNTVHRHEPPVTAEPIRLLAENEDVVVVDKPSSIPVHPCGR
FRHNTVIFILGKEHQLKELHPLHRLDRLTSGVLMFAKTAAVSERIHEQVRDRQLEKEYVCRV
EGEFPTEEVTCKEPILVVSYKVGVCRVDPRGKPCETVFQRLSYNGQSSVVRCRPLTGRTHQI
RVHLQFLGHPILNDPIYNSVAWGPSRGRGGYIPKTNEELLRDLVAEHQAKQSLDVLDLCEGD
LSPGLTDSTAPSSELGKDDLEELAAAAQKMEEVAEAAPQELDTIALASEKAVETDVMNQETD
PLCAECRLVRQDPLPQDLVMFLHALRYKGPGFEYFSPMPAWAQDDWQKD

### II. Targeting Elements

In certain aspects, provided herein are targeting elements for direction of an enzyme capable of pseudouridine installation in an RNA molecule to a specific RNA molecule. In some aspects, targeting elements comprise polypeptides, proteins, and/or RNA molecules. In some aspects, targeting elements may be endogenous proteins, polypeptides, and/or RNA molecules. In some aspects, targeting elements may be non-endogenous and/or engineered proteins, polypeptides, and/or RNA molecules.

### A. Targeting Proteins and Polypeptides

In certain aspects, enzymes capable of pseudouridine installation and/or polypeptides derived therefrom are guided to a target RNA molecule by a targeting element comprising a protein, polypeptide, and/or RNA molecule. In some aspects, a targeting element comprising a protein and/or polypeptide can be guided to a target RNA element by a complementary, or at least partially complementary, RNA molecule.

In some aspects, a targeting element comprises an RNA-guided protein. In some aspects, an RNA-guided protein is a protein that forms an RNA-protein complex and is guided to a target polynucleotide through at least partial complementarity between the RNA component & target polynucleotide. In some aspects, an RNA-guided protein comprises, consists essentially of, or consists of a Cas protein. In some aspects, a targeting element comprises a Cas protein. In some aspects, a targeting element comprises an engineered Cas protein with reduced and/or absent gene-editing activity (e.g., endonuclease activity) relative to non-engineered Cas proteins, such proteins may be referenced as “dead” Cas (dCas) proteins. In some aspects, dCas proteins such as but not limited to dCas13d, can have less than or equal to 2, 3, 4, 5, 6, 7, 8, 9, 10, 11, or 12 fold activity, or any range derivable therein, less potent gene-editing activity relative to non-engineered parental Cas proteins. In some aspects, a Cas protein can be, but is not limited to, a wild type and/or modified variant of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cas13a, Cas13b, Cas13c, Cas13d, homologs thereof, or modified versions thereof. These enzymes are known; for example, the amino acid sequence of S. pyogenes Cas9 protein may be found in the SwissProt database under accession number Q99ZW2. In some aspects, a Cas protein can be, or can be derived from, a type I, type II, type III, type IV, type V, and/or type VI, CRISPR systems.

In some aspects, a targeting element, such as a targeting protein, can be linked to a PUS protein with enzymatic activity (e.g., a PUS enzyme). In some aspects, a targeting protein can be directly fused to a PUS protein. In some aspects, a targeting protein can be linked to a PUS protein via a linker. In some aspects, a linker comprises a polypeptide. In some aspects, a linker polypeptide comprises or is a flexible polypeptide. In some aspects, a linker polypeptide is derived from XTEN. In some aspects, a linker polypeptide comprises, consists essentially of, or consists of a polypeptide that has at least or equal to exactly or about 76%, 84%, 92% or 100% sequence identity to amino acid sequence SESATPES (SEQ ID NO: 39).

In some aspects, a targeting element comprises, consists essentially of, or consists of an amino acid sequence or is encoded by a polynucleotide sequence, with at least or equal to, exactly or about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any percentage derivable therein, identity to any one of SEQ ID NOs: 19-20.

SEQ ID NO: 19-Exemplary catalytically dead Cas13d (dCas13d) polynucleotide sequence
(SEQ ID NO: 19)
ATCGAAAAAAAAAAGTCCTTCGCCAAGGGCATGGGCGTGAAGTCCACACTCGTGTCCGGCTC
CAAAGTGTACATGACAACCTTCGCCGAAGGCAGCGACGCCAGGCTGGAAAAGATCGTGGAGG
GCGACAGCATCAGGAGCGTGAATGAGGGCGAGGCCTTCAGCGCTGAAATGGCCGATAAAAAC
GCCGGCTATAAGATCGGCAACGCCAAATTCAGCCATCCTAAGGGCTACGCCGTGGTGGCTAA
CAACCCTCTGTATACAGGACCCGTCCAGCAGGATATGCTCGGCCTGAAGGAAACTCTGGAAA
AGAGGTACTTCGGCGAGAGCGCTGATGGCAATGACAATATTTGTATCCAGGTGATCCATAAC
ATCCTGGACATTGAAAAAATCCTCGCCGAATACATTACCAACGCCGCCTACGCCGTCAACAA
TATCTCCGGCCTGGATAAGGACATTATTGGATTCGGCAAGTTCTCCACAGTGTATACCTACG
ACGAATTCAAAGACCCCGAGCACCATAGGGCCGCTTTCAACAATAACGATAAGCTCATCAAC
GCCATCAAGGCCCAGTATGACGAGTTCGACAACTTCCTCGATAACCCCAGACTCGGCTATTT
CGGCCAGGCCTTTTTCAGCAAGGAGGGCAGAAATTACATCATCAATTACGGCAACGAATGCT
ATGACATTCTGGCCCTCCTGAGCGGACTGGCGCACTGGGTGGTCGCTAACAACGAAGAAGAG
TCCAGGATCTCCAGGACCTGGCTCTACAACCTCGATAAGAACCTCGACAACGAATACATCTC
CACCCTCAACTACCTCTACGACAGGATCACCAATGAGCTGACCAACTCCTTCTCCAAGAACT
CCGCCGCCAACGTGAACTATATTGCCGAAACTCTGGGAATCAACCCTGCCGAATTCGCCGAA
CAATATTTCAGATTCAGCATTATGAAAGAGCAGAAAAACCTCGGATTCAATATCACCAAGCT
CAGGGAAGTGATGCTGGACAGGAAGGATATGTCCGAGATCAGGAAAAATCATAAGGTGTTCG
ACTCCATCAGGACCAAGGTCTACACCATGATGGACTTTGTGATTTATAGGTATTACATCGAA
GAGGATGCCAAGGTGGCTGCCGCCAATAAGTCCCTCCCCGATAATGAGAAGTCCCTGAGCGA
GAAGGATATCTTTGTGATTAACCTGAGGGGCTCCTTCAACGACGACCAGAAGGATGCCCTCT
ACTACGATGAAGCTAATAGAATTTGGAGAAAGCTCGAAAATATCATGCACAACATCAAGGAA
TTTAGGGGAAACAAGACAAGAGAGTATAAGAAGAAGGACGCCCCTAGACTGCCCAGAATCCT
GCCCGCTGGCCGTGATGTTTCCGCCTTCAGCAAACTCATGTATGCCCTGACCATGTTCCTGG
ATGGCAAGGAGATCAACGACCTCCTGACCACCCTGATTAATAAATTCGATAACATCCAGAGC
TTCCTGAAGGTGATGCCTCTCATCGGAGTCAACGCTAAGTTCGTGGAGGAATACGCCTTTTT
CAAAGACTCCGCCAAGATCGCCGATGAGCTGAGGCTGATCAAGTCCTTCGCTAGAATGGGAG
AACCTATTGCCGATGCCAGGAGGGCCATGTATATCGACGCCATCCGTATTTTAGGAACCAAC
CTGTCCTATGATGAGCTCAAGGCCCTCGCCGACACCTTTTCCCTGGACGAGAACGGAAACAA
GCTCAAGAAAGGCAAGCACGGCATGAGAAATTTCATTATTAATAACGTGATCAGCAATAAAA
GGTTCCACTACCTGATCAGATACGGTGATCCTGCCCACCTCCATGAGATCGCCAAAAACGAG
GCCGTGGTGAAGTTCGTGCTCGGCAGGATCGCTGACATCCAGAAAAAACAGGGCCAGAACGG
CAAGAACCAGATCGACAGGTACTACGAAACTTGTATCGGAAAGGATAAGGGCAAGAGCGTGA
GCGAAAAGGTGGACGCTCTCACAAAGATCATCACCGGAATGAACTACGACCAATTCGACAAG
AAAAGGAGCGTCATTGAGGACACCGGCAGGGAAAACGCCGAGAGGGAGAAGTTTAAAAAGAT
CATCAGCCTGTACCTCACCGTGATCTACCACATCCTCAAGAATATTGTCAATATCAACGCCA
GGTACGTCATCGGATTCCATTGCGTCGAGCGTGATGCTCAACTGTACAAGGAGAAAGGCTAC
GACATCAATCTCAAGAAACTGGAAGAGAAGGGATTCAGCTCCGTCACCAAGCTCTGCGCTGG
CATTGATGAAACTGCCCCCGATAAGAGAAAGGACGTGGAAAAGGAGATGGCTGAAAGAGCCA
AGGAGAGCATTGACAGCCTCGAGAGCGCCAACCCCAAGCTGTATGCCAATTACATCAAATAC
AGCGACGAGAAGAAAGCCGAGGAGTTCACCAGGCAGATTAACAGGGAGAAGGCCAAAACCGC
CCTGAACGCCTACCTGAGGAACACCAAGTGGAATGTGATCATCAGGGAGGACCTCCTGAGAA
TTGACAACAAGACATGTACCCTGTTCGCCAACAAGGCCGTCGCCCTGGAAGTGGCCAGGTAT
GTCCACGCCTATATCAACGACATTGCCGAGGTCAATTCCTACTTCCAACTGTACCATTACAT
CATGCAGAGAATTATCATGAATGAGAGGTACGAGAAAAGCAGCGGAAAGGTGTCCGAGTACT
TCGACGCTGTGAATGACGAGAAGAAGTACAACGATAGGCTCCTGAAACTGCTGTGTGTGCCT
TTCGGCTACTGTATCCCCAGGTTTAAGAACCTGAGCATCGAGGCCCTGTTCGATAGGAACGA
GGCCGCCAAGTTCGACAAGGAGAAAAAGAAGGTGTCCGGCAATTCC
SEQ ID NO: 20-Exemplary catalytically dead Cas13d (dCas13d) amino acid sequence
(SEQ ID NO: 20)
IEKKKSFAKGMGVKSTLVSGSKVYMTTFAEGSDARLEKIVEGDSIRSVNEGEAFSAEMADKN
AGYKIGNAKFSHPKGYAVVANNPLYTGPVQQDMLGLKETLEKRYFGESADGNDNICIQVIHN
ILDIEKILAEYITNAAYAVNNISGLDKDIIGFGKFSTVYTYDEFKDPEHHRAAFNNNDKLIN
AIKAQYDEFDNFLDNPRLGYFGQAFFSKEGRNYIINYGNECYDILALLSGLAHWVVANNEEE
SRISRTWLYNLDKNLDNEYISTLNYLYDRITNELTNSFSKNSAANVNYIAETLGINPAEFAE
QYFRFSIMKEQKNLGFNITKLREVMLDRKDMSEIRKNHKVFDSIRTKVYTMMDFVIYRYYIE
EDAKVAAANKSLPDNEKSLSEKDIFVINLRGSENDDQKDALYYDEANRIWRKLENIMHNIKE
FRGNKTREYKKKDAPRLPRILPAGRDVSAFSKLMYALTMELDGKEINDLLTTLINKEDNIOS
FLKVMPLIGVNAKFVEEYAFFKDSAKIADELRLIKSFARMGEPIADARRAMYIDAIRILGIN
LSYDELKALADTFSLDENGNKLKKGKHGMRNFIINNVISNKRFHYLIRYGDPAHLHEIAKNE
AVVKFVLGRIADIQKKQGQNGKNQIDRYYETCIGKDKGKSVSEKVDALTKIITGMNYDQFDK
KRSVIEDTGRENAEREKFKKIISLYLTVIYHILKNIVNINARYVIGFHCVERDAQLYKEKGY
DINLKKLEEKGFSSVTKLCAGIDETAPDKRKDVEKEMAERAKESIDSLESANPKLYANYIKY
SDEKKAEEFTRQINREKAKTALNAYLRNTKWNVIIREDLLRIDNKTCTLFANKAVALEVARY
VHAYINDIAEVNSYFQLYHYIMQRIIMNERYEKSSGKVSEYFDAVNDEKKYNDRLLKLLCVP
FGYCIPREKNLSIEALEDRNEAAKEDKEKKKVSGNS

### B. Targeting RNAs

In certain aspects, provided herein are targeting RNAs that may guide a protein and/or ribonucleoprotein complex to a target RNA of interest. In some aspects, a targeting RNA is an engineered RNA.

In certain aspects, a targeting RNA is driven by a promoter comprising a Polymerase III promoter (i.e., a promoter that can drive Pol III mediated transcription). In certain aspects, transcription of a targeting RNA is driven by one or more U6 promoters. In certain aspects, a targeting RNA is transcribed by Polymerase III. In certain aspects, a targeting RNA is driven by a promoter comprising a Polymerase II promoter (i.e., a promoter that can drive Pol II mediated transcription). In some aspects, a targeting RNA is designed to be comprised in an intronic sequence.

In certain aspects, a targeting RNA is an engineered small nucleolar RNA (snoRNA) designed to target a user-defined RNA locus. In certain aspects, a targeting RNA is an engineered box H/ACA RNA. In some aspects, a targeting RNA can guide an endogenous protein and/or ribonucleoprotein complex to a target RNA of interest. In some aspects, a targeting RNA can guide a snoRNA-DKC1 system. In some aspects, a targeting RNA can guide a box H/ACA ribonucleoprotein complex (e.g., comprising a box H/ACA RNA, Nhp2, Nop10, Gar1, and the pseudouridylase Cbf5 (also known as DKC1)).

In some aspects, provided herein are CRISPR/Cas system ancillary components, such as functional targeting RNA species, for example but not limited to, CRISPR RNA, trans-activating CRISPR RNA (tracrRNA), and/or gRNA.

In some aspects, provided herein are targeting RNA species that mediate greater than or equal to, exactly or about 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%2, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, pseudouridine installation in a target polynucleotide population. In some aspects, targeting RNA species comprise gRNA molecules. In some aspects, a gRNA molecule is engineered to provide improved functionality relative to a non-engineered gRNA.

In some aspects, an RNA targeting element comprises a gRNA sequence, which may comprise or consist of a polynucleotide sequence, with at least or equal to, exactly or about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any percentage derivable therein, identity to any one of SEQ ID NOs: 21-28 and 35-45.

SEQ ID NO: 21-Exemplary ERH targeting guide RNA polynucleotide sequence
(SEQ ID NO: 21)
TTAAGTGACGAAGAACAACTTC
SEQ ID NO: 22-Exemplary SCP2 targeting guide RNA polynucleotide sequence
(SEQ ID NO: 22)
AATTTGATGAGTTCTGGCGGCT
SEQ ID NO: 23-Exemplary AMFR targeting guide RNA polynucleotide sequence
(SEQ ID NO: 23)
GTTCCTTTTCCAAGATGCCCTG
SEQ ID NO: 24-Exemplary CDC6 targeting guide RNA polynucleotide sequence
(SEQ ID NO: 24)
ATTGTCCAGAACCTGATCTCTA
SEQ ID NO: 25-Exemplary KRAS U62 (aka ATA1) targeting guide RNA polynucleotide
sequence
(SEQ ID NO: 25)
GGCACTCTTGCCTACGCCACCA
SEQ ID NO: 26-Exemplary KRAS U107 (aka ATA2) targeting guide RNA polynucleotide
sequence
(SEQ ID NO: 26)
GATCATATTCGTCCACAAAATGA
SEQ ID NO: 27-Exemplary KRAS U251 (aka ATA3) targeting guide RNA polynucleotide
sequence
(SEQ ID NO: 27)
GTACACAAAGAAAGCCCTCCCCA
SEQ ID NO: 28-Exemplary KRAS U563 (aka ATA4) targeting guide RNA polynucleotide
sequence
(SEQ ID NO: 28)
GTTTTTTAATTTTCACACAGCCA
SEQ ID NO: 35-Exemplary p53 guide RNA “G2” polynucleotide sequence
(SEQ ID NO: 35)
AAACGTTTCTGTCATCCAAATACTCCA
SEQ ID NO: 36-Exemplary control snoRNA polynucleotide sequence
(SEQ ID NO: 36)
TCAGCCACGAAAGCCTGCACCTGACCAGGTCTCTGTTGGCTGGTGCAACCCCAGGGTGAGCT
GATAGTAAACCCCAGCTAATTTGACAGGGTTGTTCTTCATGTGGATGACTCTGTCTCTAAAG
CATGGGAACAGCC
SEQ ID NO: 37-Exemplary KRAS U62-U107 snoRNA polynucleotide sequence
(SEQ ID NO: 37)
TCAGCCACTTAGCTGTGCACCTGACCAGGTCTCTGTTGGCTGGTGCAATCGTCAGGTGAGCT
GATAGTAAACCCCAGCTATCCTCACAGGGTTGTTCTTCATGTGGATGACTCTGTTTGTTAAG
CATGGGAACAGCC
SEQ ID NO: 38-Exemplary KRAS U251-U563 snoRNA polynucleotide sequence
(SEQ ID NO: 38)
TCAGCCACTATTATTTGCACCTGACCAGGTCTCTGTTGGCTGGTGCAATGGCAAGGTGAGCT
GATAGTAAACCCCAGCTTTACATACAGGGTTGTTCTTCATGTGGATGACTCTGTTTTATAAG
CATGGGAACAGCC
SEQ ID NO: 40-Exemplary CFTR U1616-NULL snoRNA polynucleotide sequence
(SEQ ID NO: 40)
TCAGCCACcaagaacTGCACCTGACCAGGTCTCTGTTGGCTGGTGCAAtattgtGGTGAGCT
GATAGTAAACCCCAGCTgagggtACAGGGTTGTTCTTCATGTGGATGACTCTGTaggtgAAG
CATGGGAACAGCC
SEQ ID NO: 41-Exemplary KRAS A227 snoRNA polynucleotide sequence
(SEQ ID NO: 41)
TCAGCCACgaaagccTGCACCTGACCAGGTCTCTGTTGGCTGGTGCAAccccagGGTGAGCT
GATAGTAAACCCCAGCTaaagccACAGGGTTGTTCTTCATGTGGATGACTCTGTccccaAAG
CATGGGAACAGCC
SEQ ID NO: 42-Exemplary KRAS U68 snoRNA polynucleotide sequence
(SEQ ID NO: 42)
TCAGCCACtctgaatTGCACCTGACCAGGTCTCTGTTGGCTGGTGCAAgctgtaGGTGAGCT
GATAGTAAACCCCAGCTctgaatACAGGGTTGTTCTTCATGTGGATGACTCTGTgctgtAAG
CATGGGAACAGCC
SEQ ID NO: 43-Exemplary KRAS U62 snoRNA polynucleotide sequence
(SEQ ID NO: 43)
TCAGCCACttagctgTGCACCTGACCAGGTCTCTGTTGGCTGGTGCAAtcgtcaGGTGAGCT
GATAGTAAACCCCAGCTtagctgACAGGGTTGTTCTTCATGTGGATGACTCTGTtcgtcAAG
CATGGGAACAGCC
SEQ ID NO: 44-Exemplary KRAS U107 snoRNA polynucleotide sequence
(SEQ ID NO: 44)
TCAGCCACaatcctcTGCACCTGACCAGGTCTCTGTTGGCTGGTGCAAttgttgGGTGAGCT
GATAGTAAACCCCAGCTatcctcACAGGGTTGTTCTTCATGTGGATGACTCTGTttgttAAG
CATGGGAACAGCC
SEQ ID NO: 45-Exemplary KRAS U251 snoRNA polynucleotide sequence
(SEQ ID NO: 45)
TCAGCCACtattattTGCACCTGACCAGGTCTCTGTTGGCTGGTGCAAtggcaaGGTGAGCT
GATAGTAAACCCCAGCTattattACAGGGTTGTTCTTCATGTGGATGACTCTGTtggcaAAG
CATGGGAACAGCC

### C. In Vitro Transcription

In some aspects, an RNA of the disclosure may be in vitro transcribed. In some aspects, gRNA, and/or snoRNA are in vitro transcribed. In some aspects, an in vitro transcription kit may be utilized to produce an in vitro transcribed RNA (e.g., MEGAshortscript™ T7 Transcription Kit (#AM1354, Fisher Scientific).

In the art, straight-forward processes for the recombinant production of RNA molecules in preparative amounts have been developed in a process called “RNA in vitro transcription”. The term “RNA in vitro transcription” relates to a process wherein RNA is synthesized in a cell-free system (in vitro). RNA is commonly obtained by enzymatic DNA dependent in vitro transcription of an appropriate DNA template, which is often a linearized plasmid DNA template. The promoter for controlling RNA in vitro transcription can be any promoter for any DNA dependent RNA polymerase. Particular examples of DNA dependent RNA polymerases are the bacteriophage enzymes T7, T3, and/or SP6 RNA polymerases.

Methods for RNA in vitro transcription are known in the art (see for example Geall et al. (2013) Semin. Immunol. 25(2): 152-159; Brunelle et al. (2013) Methods Enzymol. 530: 101-14). Reagents used in said methods may include: a linear DNA template with a promoter sequence that has a high binding affinity for its respective RNA polymerase; ribonucleoside triphosphates (NT) for the four bases (adenine, cytosine, guanine and uracil); a cap analog (for example, but not limited to, m7G(5′)ppp(5′)G (m7G)); other modified nucleotides; DNA-dependent RNA polymerase (for example, but not limited to, T7, T3 or SP6 RNA polymerase); ribonuclease (RNase) inhibitor to inactivate any contaminating RNase; pyrophosphatase to degrade pyrophosphate, which inhibits transcription; MgCl₂, which supplies Mg²⁺ as a cofactor for the RNA polymerase; antioxidants (for example, but not limited to, DTT); polyamines such as spermidine; and a buffer to maintain a suitable pH value.

Common buffer systems used in RNA in vitro transcription include 4-(2-hydroxy-ethyl)-1-piperazineethanesulfonic acid (HEPES) and tris(hydroxymethyl) amino-methane (Tris). The pH value of the buffer is commonly adjusted to a pH value of about 6 to 8.5. Some commonly used transcription buffers comprise 80 mM HEPES/KOH, pH 7.5 and 40 mM Tris/HCl, pH 7.5.

The transcription buffer can also contain a magnesium salt such as MgCl₂ commonly in a range between 5-50 mM. Magnesium ions (Mg²⁺) are an essential component in an RNA in vitro transcription buffer system because free Mg²⁺ acts as cofactor in the catalytic center of the RNA polymerase and is critical for the RNA polymerization reaction. In diffuse binding, fully hydrated Mg²⁺ ions also interact with the RNA product via nonspecific long-range electrostatic interactions.

RNA in vitro transcription reactions are typically performed as batch reactions in which all components are combined and then incubated to allow the synthesis of RNA molecules until the reaction terminates. In addition, fed-batch reactions were developed to increase the efficiency of the RNA in vitro transcription reaction (see e.g., Kern et al. (1997) Biotechnol. Prag. 13: 747-756; and Kern et al. (1999) Biotechnol. Prog. 15: 174-184, all of which are incorporated herein by reference in their entirety). In a fed-batch system, all components are combined, but then additional amounts of some of the reagents are added over time (for example, but not limited to, NT, and/or MgCl₂) to maintain constant reaction conditions.

Moreover, the use of a bioreactor (transcription reactor) for the synthesis of RNA molecules by in vitro transcription has been reported (see e.g., WO 1995/08626, incorporated herein by reference in its entirety). The bioreactor is configured such that reactants are delivered via a feed line to the reactor core and RNA products are removed by passing through an ultrafiltration membrane (having a nominal molecular weight cut-off, for example, but not limited to, 100,000 Daltons) to the exit stream.

The concentration of the nucleic acid template comprised in the in vitro transcription mixture is in a range from about 1 to 50 nM, 1 to 40 nM, 1 to 30 nM, 1 to 20 nM, or about 1 to 10 nM. In certain aspects, the concentration of the nucleic acid template is from about 10 to 30 nM. In certain aspects, the concentration of the nucleic acid template is about 20 nM. In certain aspects, a concentration of the nucleic acid template is of about 1 to 200 g/ml, about 10 to 100 g/ml, or about 20 to 50 g/ml.

RNA POLYMERASE: The RNA polymerase is an enzyme which catalyzes the transcription of a DNA template into RNA. In certain aspects, suitable RNA polymerases for use in methods and/or compositions of the present disclosure include, but are not limited to, T7, T3, SP6 and E. coli RNA polymerase. In certain aspects, a T7 RNA polymerase is used. In certain aspects, an RNA polymerase for use in the present disclosure is a recombinant RNA polymerase, meaning that it is added to the RNA in vitro transcription reaction as a single component and not as part of a cell extract which contains other components in addition to the RNA polymerase. The skilled person knows that the choice of the RNA polymerase depends on the promoter present in the DNA template which has to be bound by the suitable RNA polymerase. In certain aspects, the concentration of the RNA polymerase is from about 1 to 100 nM, 1 to 90 nM, 1 to 80 nM, 1 to 70 nM, 1 to 60 nM, 1 to 50 nM, 1 to 40 nM, 1 to 30 nM, 1 to 20 nM, or about 1 to 10 nM. In certain aspects, the concentration of the RNA polymerase is from about 10 to 50 nM, 20 to 50 nM, or 30 to 50 nM. In certain aspects, the RNA polymerase concentration is about 40 nM. In certain aspects, a concentration of 500 to 10000 U/ml of the RNA polymerase is used. In certain aspects, a concentration of 1000 to 7500 U/ml, or a concentration of 2500 to 5000 Units/ml of the RNA polymerase is used. The person skilled in the art will understand that the choice of the RNA polymerase concentration is influenced by the concentration of the DNA template.

### D. Transfection

As used herein, the term “transfection” is defined as the introduction of an extracellular nucleic acid into a host cell by any means known in the art, including, but not limited to, calcium phosphate co-precipitation, viral transduction, liposome fusion, microinjection, microparticle bombardment, electroporation. The terms “uptake of nucleic acid by a host cell”, “taking up of nucleic acid by a host cell”, “uptake of particles comprising nucleic acid by a host cell”, and “taking up of particles comprising nucleic acid by a host cell” denote any process wherein an extracellular nucleic acid, with or without accompanying material, enters a host cell.

A variety of methods are known in the art and suitable for transfection of nucleic acid into a cell. The polynucleotides of the present disclosure may be formulated, using the methods described herein. The formulations may comprise polynucleotides which may be modified and/or unmodified. The formulations may further comprise, but are not limited to, cell penetration agents, a pharmaceutically acceptable carrier, a delivery agent, a bioerodible or biocompatible polymer, a solvent, a sustained-release delivery depot, and/or any combinations thereof.

The formulated polynucleotides may be delivered to the cell using routes of administration known in the art and described herein. Examples of typical methods include, but are not limited to, naked delivery, lipidoid mediate transfer, liposome-, lipoplexes, and/or lipid nanoparticle-mediated transfer, electroporation, calcium phosphate mediated transfer, nucleofection, sonoporation, heat shock, magnetofection, microinjection, microprojectile mediated transfer (for example, but not limited to, nanoparticles), cationic polymer mediated transfer (for example, but not limited to, DEAE-dextran, polyethylenimine, polyethylene glycol (PEG) and the like) or cell fusion.

### E. Vectors

Among other things, the present disclosure provides that in some aspects, polypeptides and/or oligonucleotides described herein are encoded by a polynucleotide, such as a vector comprising a polynucleotide (e.g., a polynucleotide construct). Vectors comprising polynucleotide constructs according to the present disclosure include all those known in the art, including cosmids, plasmids (e.g., naked or contained in liposomes) and viral constructs (e.g., lentiviral, retroviral, adenoviral, and adeno associated viral constructs) that incorporate a polynucleotide comprising a pseudouridine synthase and/or targeting element described herein, or characteristic portions thereof (e.g., as utilized herein, a “characteristic portion thereof” refers to the portion of said protein required to perform the desired function, e.g., it comprises the ability to target and/or install a pseudouridine in a site-specific manner). Those of skill in the art will be capable of selecting suitable constructs, as well as cells, for making any of the polynucleotides described herein. In some aspects, a construct is a plasmid (i.e., a circular DNA molecule that can autonomously replicate inside a cell). In some aspects, a construct can be a cosmid (e.g., pWE or sCos series).

In some aspects, a construct is a viral construct. In some aspects, a viral construct is a lentivirus, retrovirus, adenovirus, or adeno-associated virus construct. In some aspects, a construct is an adeno-associated virus (AAV) construct (see, e.g., Asokan et al., Mol. Ther. 20: 699-7080, 2012, which is incorporated herein by reference for the purposes described herein). In some aspects, a viral construct is an adenovirus construct. In some aspects, a viral construct may also be based on or derived from an alphavirus. Alphaviruses include but are not limited to, Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, Semliki Forest virus (SFV), and Venezuelan equine encephalitis virus (VEEV) have all been used to develop viral constructs for coding sequence delivery. Pseudotyped viruses may be formed by combining alphaviral envelope glycoproteins and retroviral capsids. Examples of alphaviral constructs can be found in U.S. Publication Nos. 20150050243, 20090305344, and 20060177819; constructs and methods of their making are incorporated herein by reference for the purposes described herein.

In some aspects, constructs provided herein can be of different sizes. In some aspects, a construct is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some aspects, a construct is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some aspects, a construct is a viral construct and can have a total number of nucleotides of up to 10 kb. In some aspects, a viral construct can have a total number of nucleotides in the range of about 4.5 kb to 5 kb, or about 4.7 kb. In some aspects, a viral construct can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some aspects, a construct is a lentivirus construct and can have a total number of nucleotides of up to 8 kb. In some examples, a lentivirus construct can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8 kb, about 6 kb to about 7 kb, or about 7 kb to about 8 kb.

In some aspects, a construct is an adenovirus construct and can have a total number of nucleotides of up to 8 kb. In some aspects, an adenovirus construct can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 kb to about 8 kb.

Any of the constructs described herein can further include a control sequence, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or additional untranslated regions which may house pre- or post-transcriptional regulatory and/or control elements. In some aspects, a promoter can be a native promoter, a constitutive promoter, an inducible promoter, and/or a tissue-specific promoter. Non-limiting examples of control sequences are described herein.

### A. AAV Particles

Among other things, the present disclosure provides AAV particles that comprise a polynucleotide construct encoding a pseudouridine synthase and/or targeting element, and an AAV capsid. In some aspects, AAV particles can be described as having a serotype, which is a description of the construct strain and the capsid strain. For example, in some aspects an AAV particle may be described as AAV2, wherein the particle has an AAV2 capsid and a construct that comprises characteristic AAV2 Inverted Terminal Repeats (ITRs). In some aspects, an AAV particle may be described as a pseudotype, wherein the capsid and construct are derived from different AAV strains, for example, AAV2/9 would refer to an AAV particle that comprises a construct utilizing the AAV2 ITRs and an AAV9 capsid. Additional examples of pseudotyped AAV vectors include, but are not limited to, AAV2/1, AAV2/2, AAV2/3, AAV2/4, AAV2/5, AAV2/6, AAV2/7, AAV2/8 and AAV2/9.

In some aspects, AAV particles suitable for use according to the present disclosure may comprise or be derived from any natural or recombinant AAV serotype. In some aspects, an AAV according to the present invention is selected from natural serotypes such as AAV1, AAV2, AAV3, AAV4, AAV5, AAV6, AAV7, AAV8, AAV9, AAV10, AAV11, and AAV12; or pseudotypes, chimeras, and variants thereof.

As used herein, the term “chimera” when referring to an AAV vector, or a “chimeric AAV vector”, refers to an AAV vector which comprises a capsid containing VP1, VP2 and VP3 proteins from at least two different AAV serotypes; or alternatively, which comprises VP1, VP2 and VP3 proteins, at least one of which comprises at least a portion from another AAV serotype. Examples of chimeric AAV vectors include, but are not limited to, AAV-DJ, AAV-DJ/8, AAV2G9, AAV2i8, AAV2i8G9, AAV8G9, and AAV9i1.

In some aspects, an AAV serotype and/or pseudotype according to the present invention is selected from the group comprising or consisting of AAV1, AAV2, AAV3, AAV 4, AAV5, AAV6, AAV7, AAV8, AAV9, AAV10, AAV11, AAV12, AAV106.1/hu.37, AAV114.3/hu.40, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2i8, AAV2i8G9, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV2.5T, AAV27.3, AAV29.3/bb.1, AAV29.5/bb.2, AAV2G9, AAV3B, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/rh.64, AAV4-9/rh.54, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6.1, AAV6.1.2, AAV6.2, AAV7m8, AAV7.2, AAV7.3/hu.7, AAV-8b, AAV8G9, AAV-8h, AAV9i1, AAV9.11, AAV9.13, AAV9.16, AAV9.24, AAV9.45, AAV9.47, AAV9.61, AAV9.68, AAV9.84, AAV9.9, AAVcy.2, AAVcy.3, AAVcy.4, AAVcy.5, AAVcy.5R1, AAVcy.5R2, AAVcy.5R3, AAVcy.5R4, AAVcy.6, AAVhu.1, AAVhu.2, AAVhu.3, AAVhu.4, AAVhu.5, AAVhu.6, AAVhu.7, AAVhu.8, AAVhu.9, AAVhu.10, AAVhu.11, AAVhu.12, AAVhu.13, AAVhu.14/9, AAVhu.15, AAVhu.16, AAVhu.17, AAVhu.18, AAVhu.19, AAVhu.20, AAVhu.21, AAVhu.22, AAVhu.23.2, AAVhu.24, AAVhu.25, AVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.53, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVpi.1, AAVpi.2, AAVpi.3, AAVrh.2, AAVrh.2R, AAVrh.8, AAVrh.8R, AAVrh8R R533A mutant, AAVrh8R A586R mutant, AAVrh.10, AAVrh.12, AAVrh.13, AAVrh. 13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAV-PHP.B, AAVPHP.A, AAV-G2B-26, AAV-G2B-13, AAV-TH1 0.1-32, AAVTH1.1-35, AAV-PHP.B2, AAV-PHP.B3, AAV-PHP.N/PHP.B-DGT, AAV-PHP.B-EST, AAV-PHP.B-GGT, AAV-PHP.BATP, AAV-PHP.B-ATT-T, AAV-PHP.B-DGT-T, AAV-PHP.B-GGT-T, AAV-PHP.B-SGS, AAV-PHP.B-AQP, AAV-PHP.B-QQP, AAV-PHP.B-SNP(3), AAV-PHP.B-SNP, AAV-PHP.B-QGT, AAV-PHP.B-NQT, AAV-PHP.B-EGS, AAV-PHP.BSGN, AAV-PHP.B-EGT, AAV-PHP.B-DST, AAV-PHP.BDST, AAV-PHP.B-STP, AAV-PHP.B-PQP, AAV-PHP.BSQP, AAV-PHP.B-Q1P, AAV-PHP.B-TMP, AAV-PHP.BTTP, AAV-PHP.S/G2A12, AAV-G2A15/G2A3, AAV-G2B4, AAV-G2B5, PHP.S, AAAV, AAV A3.3, AAV A3.4, AAV A3.5, AAV A3.7, AAV CBr-7.3, AAV CBr-7.1, AAV CBr-7.10, AAV CBr-7.2, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-N4, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKdB4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-M9, AAV CLv-R6, AAV CLv-1, AAV CLv1-1, AAV CLv1-10, AAV CLv1-2, AAV CLv-12, AAV CLv1-3, AAV CLv-13, AAV CLv1-4, AAV CLv1-7, AAV CLv1-8, AAV CLv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLvM7, AAV CLv-M8, AAV CLv-R1, AAV CLv-R2, AAV CLv-R3, AAV CLv-R4, AAV CLv-R5, AAV CLv-R7, AAV CLv-R8, AAV CLv-R9, AAV CSp-8.10, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.2, AAV CSp-8.4, AAV CSp-8.5, AAV CSp-8.6, AAV CSp-8.7, AAV CSp-8.8, AAV CSp-8.9, AAV CSp-9, AAVLK08, AAV-LK15, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, AAV SM 10-8, AAV.VR-355, AAV-b, AAVC1, AAVC2, AAVC5, AAVCh.5, AAVCh.5R1, AAV-DJ, AAV-DJ8, AAVF1/HSC1, AAVF11/HSC11, AAVF12/HSC12, AAVF13/HSC13, AAVF14/HSC14, AVF15/HSC15, AAVF16/HSC16, AAVF17/HSC17, AAVF2/HSC2, AAVF3, AAVF3/HSC3, AAVF4/HSC4, AAVF5, AAVF5/HSC5, AAVF6/HSC6, AAVF7/HSC7, AAVF8/HSC8, AAVF9/HSC9, AAV-h, AAVH-1/hu.1, AAVH2, AAVH-5/hu.3, AAVH6, AAVhE1.1, AAVhEr1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVLG-10/rh.40, AAVLG-4/rh.38, AAVLG-9/hu.39, AAVLG-9/hu.39, AAV-LK01, AAV-LK02, AAV-LK03, AAV-LK03, AAV-LK04, AAV-LK05, AAV-LK06, AAVLK07, AAV-LK09, AAV-LK10, AAV-LK11, AAV-LK12, AAV-LK13, AAV-LK14, AAV-LK16, AAV-LK17, AAVLK18, AAV-LK19, AAVN721-8/rh.43, AAV-PAEC, AAVPAEC12, AAV-PAEC11, AAV-PAEC2, AAV-PAEC4, AAVPAEC6, AAV-PAEC7, AAV-PAECS, Anc80, Anc80L65, Anc81, Anc82, Anc83, Anc84, Anc94, Anc110, Anc113, Anc126, Anc127, BAAV, BNP61 AAV, BNP62 AAV, BNP63 AAV, bovine AAV, caprine AAV, Japanese AAV10 serotype, UPENN AAV10, VOY101, and VOY201.

In some aspects, an AAV is an AAV variant that has been genetically modified, e.g., by substitution, deletion or addition of one or several amino acid residues in one or more capsid proteins. Examples of such variants include, but are not limited to, AAV2 with one or more of Y444F, Y500F, Y730F and/or S662V mutations; AAV3 with one or more of Y705F, Y731F and/or T492V mutations; and AAV6 with one or more of S663V and/or T492V mutations.

In some aspects, an AAV capsid is modified to comprise at least one surface-bound saccharide or a derivative thereof. As used herein, the term “surface-bound”, when referring to the at least one saccharide, means that said at least one saccharide is bound to and exposed at the outer surface of the AAV vector. Suitable examples of saccharides include, but are not limited to, monosaccharides, oligosaccharides, polysaccharides, and derivatives thereof.

### B. AAV Constructs

In some aspects, the present disclosure provides polynucleotide vectors (e.g., polynucleotide constructs) that comprise a nucleotide sequence encoding a pseudouridine synthase and/or targeting element. In some aspects described herein, a polynucleotide vector comprising a nucleotide sequence encoding a pseudouridine synthase and/or targeting element, can be comprised in an AAV capsid to produce an AAV particle (e.g., an AAV particle comprises an AAV construct comprised in an AAV capsid).

In some aspects, a polynucleotide construct comprises one or more components derived from or modified from a naturally occurring AAV genomic construct. In some aspects, a sequence derived from an AAV construct is an AAV1 construct, an AAV2 construct, an AAV3 construct, an AAV4 construct, an AAV5 construct, an AAV6 construct, an AAV7 construct, an AAV8 construct, an AAV DJ/8 construct, an AAV9 construct, an AAV2.7m8 construct, an AAV8BP2 construct, an AAV293 construct, an AAVPhp.B construct, or AAVPhp.eB construct (see e.g., Chan et al., 2017). Additional exemplary AAV constructs that can be used herein are known in the art. See, e.g., Kanaan et al., Mol. Ther. Nucleic Acids 8: 184-197, 2017; Li et al., Mol. Ther. 16(7): 1252-1260, 2008; Adachi et al., Nat. Commun. 5: 3075, 2014; Isgrig et al., Nat. Commun. 10(1): 427, 2019; and Gao et al., J. Virol. 78(12): 6381-6388, 2004; each of which are incorporated herein by reference for the purposes described herein).

In some aspects, AAV derived sequences (e.g., which are comprised in a polynucleotide construct) typically include the cis-acting 5′ and 3′ ITR sequences (see, e.g., B. J. Carter, in “Handbook of Parvoviruses,” ed., P. Tijsser, CRC Press, pp. 155 168, 1990, which is incorporated herein by reference for the purposes described herein). Typical AAV2-derived ITR sequences are about 145 nucleotides in length. In some aspects, at least or exactly 80% of a typical ITR sequence (e.g., at least or exactly 85%, at least or exactly 90%, at least or exactly 95%, or at least or exactly 100%, etc.) is incorporated into a construct provided herein. The ability to modify these ITR sequences is within the skill of the art. (See, e.g., texts such as Sambrook et al., “Molecular Cloning. A Laboratory Manual”, 2d ed., Cold Spring Harbor Laboratory, New York, 1989; and K. Fisher et al., J Virol. 70:520 532, 1996, each of which is incorporated herein by reference for the purposes described herein). In some aspects, any of the coding sequences and/or constructs described herein are flanked by 5′ and 3′ AAV ITR sequences. The AAV ITR sequences may be obtained from any known AAV, including presently identified AAV types.

In some aspects, polynucleotide constructs described in accordance with this disclosure and in a pattern known to the art (see, e.g., Asokan et al., Mal. Ther. 20: 699-7080, 2012, which is incorporated herein by reference for the purposes described herein) are typically comprised of, a coding sequence or a portion thereof, at least one and/or control sequence, and optionally 5′ and 3′ AAV inverted terminal repeats (ITRs). In some aspects, provided constructs can be packaged into a capsid to create an AAV particle. An AAV particle may be delivered to a selected target cell. In some aspects, provided constructs comprise an additional optional coding sequence that is a nucleic acid sequence (e.g., inhibitory nucleic acid sequence), heterologous to the construct sequences, which encodes a polypeptide, protein, functional RNA molecule (e.g., miRNA, miRNA inhibitor) or other gene product, of interest. In some aspects, a nucleic acid coding sequence is operatively linked to and/or control components in a manner that permits coding sequence transcription, translation, and/or expression in a cell of a target tissue.

In some aspects, an unmodified AAV endogenous genome includes two open reading frames, “cap” and “rep,” which are flanked by ITRs. In some aspects, recombinant AAV constructs similarly comprise one or more open reading frames flanked by ITR sequences. In some aspects, an AAV construct also comprises conventional control elements that are operably linked to the coding sequence in a manner that permits its transcription, translation and/or expression in a cell transfected with the polynucleotide construct or infected with a virus particle produced by the disclosure. In some aspects, an AAV construct optionally comprises a promoter, an enhancer, an untranslated region (e.g., a 5′ UTR, 3′ UTR), a Kozak sequence, an internal ribosomal entry site (IRES), splicing sites (e.g., an acceptor site, a donor site), a polyadenylation site, or any combination thereof.

In some aspects, a construct is an AAV construct. In some aspects, an AAV construct can include at least 500 bp, at least 1 kb, at least 1.5 kb, at least 2 kb, at least 2.5 kb, at least 3 kb, at least 3.5 kb, at least 4 kb, at least 4.5 kb, or at least 4.7 kb. In some aspects, an AAV construct can include at most 7.5 kb, at most 7 kb, at most 6.5 kb, at most 6 kb, at most 5.5 kb, at most 5 kb, at most 4.5 kb, at most 4 kb, at most 3.5 kb, at most 3 kb, or at most 2.5 kb. In some aspects, an AAV construct can include about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, or about 4 kb to about 5 kb.

Any of the constructs described herein can further include regulatory and/or control sequences, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or any combination thereof. In some aspects, a promoter can be a native promoter, a constitutive promoter, an inducible promoter, and/or a tissue-specific promoter. Non-limiting examples of control sequences are described herein and others are known in the art

### C. AAV Capsids

In some aspects, the present disclosure provides one or more polynucleotide constructs packaged into an AAV capsid. In some aspects, an AAV capsid is from or is derived from an AAV capsid of an AAV2, 3, 4, 5, 6, 7, 8, 9, 10, rh8, rh10, rh39, rh43 or Ancestral serotype, or one or more hybrids thereof. In some aspects, an AAV capsid is from an AAV ancestral serotype. In some aspects, an AAV capsid is an ancestral (Anc) AAV capsid. An Anc capsid is created from a construct sequence that is constructed using evolutionary probabilities and evolutionary modeling to determine a probable ancestral sequence. Thus, an Anc capsid/construct sequence is not known to have existed in nature. As provided herein, in some aspects, any combination of AAV capsids and AAV constructs (e.g., comprising AAV ITRs) may be used in recombinant AAV particles of the present disclosure.

### D. Exemplary AAV Construct Components

### 1. Inverted Terminal Repeat Sequences (ITRs)

AAV derived sequences of a construct typically comprises the cis-acting 5′ and 3′ ITRs (See, e.g., B. J. Carter, in “Handbook of Parvoviruses”, ed., P. Tijsser, CRC Press, pp. 155 168 (1990), which is incorporated herein by reference for the purposes described herein). Generally, ITRs are able to form a hairpin. The ability to form a hairpin can contribute to an ITRs ability to self-prime, allowing primase-independent synthesis of a second DNA strand. ITRs can also aid in efficient encapsidation of an AAV construct in an AAV particle.

An AAV particle of the present disclosure can comprise an AAV construct comprising a coding sequence (e.g., encoding a pseudouridine synthase and/or targeting element) and associated elements flanked by a 5′ and a 3′ AAV ITR sequences. In some aspects, an ITR is or comprises about 130 nucleic acids. In some aspects, an ITR is or comprises about 145 nucleic acids. In some aspects, all or substantially all of a sequence encoding an ITR is used. In some aspects, an AAV ITR sequence may be obtained from any known AAV, including presently identified mammalian AAV types. In some aspects an ITR is an AAV2 ITR. In some aspects, an ITR is an AAV9 ITR.

A non-limiting example of a polynucleotide construct of the present disclosure is a “cisacting” construct comprising a coding sequence, in which said sequence and any associated regulatory elements are flanked by 5′ or “left” and 3′ or “right” AAV ITR sequences. 5′ and left designations refer to a position of an ITR sequence relative to an entire construct, read left to right, in a sense direction. For example, in some aspects, a 5′ or left ITR is an ITR that is closest to a promoter (e.g., as opposed to a polyadenylation sequence) for a given construct, when a construct is depicted in a sense orientation, linearly. Concurrently, 3′ and right designations refer to a position of an ITR sequence relative to an entire construct, read left to right, in a sense direction. For example, in some aspects, a 3′ or right ITR is an ITR that is closest to a polyadenylation sequence and/or stop codon (e.g., as opposed to a promoter sequence) for a given construct, when a construct is depicted in a sense orientation, linearly. In general, ITRs as provided herein are depicted in 5′ to 3′ order in accordance with a sense strand. Accordingly, one of skill in the art will appreciate that a 5′ or “left” orientation ITR can also be depicted as a 3′ or “right” ITR when converting from sense to anti sense direction. Further, it is well within the ability of one of skill in the art to transform a given sense ITR sequence (e.g., a 5/left AAV ITR) into an antisense sequence (e.g., 3′/right ITR sequence). One of ordinary skill in the art would understand how to modify a given ITR sequence for use as either a 5/left or 3′/right ITR, or an antisense version thereof.

### 2. Promoters

In some aspects, a construct (e.g., an AAV construct) comprises a promoter. The term “promoter” refers to a DNA sequence recognized by enzymes/proteins that can promote and/or initiate transcription of an operably linked gene. For example, a promoter typically refers to, e.g., a nucleotide sequence to which an RNA polymerase and/or any associated factor binds and from which it can initiate transcription. Thus, in some aspects, a construct (e.g., an AAV construct) comprises a promoter operably linked to one of the non-limiting example promoters described herein.

In some aspects, a promoter is an inducible promoter, a constitutive promoter, a mammalian cell promoter, a viral promoter, a chimeric promoter, an engineered promoter, a tissue-specific promoter, or any other type of promoter known in the art. In some aspects, a promoter is a RNA polymerase II promoter, such as a mammalian RNA polymerase II promoter. In some aspects, a promoter is a RNA polymerase III promoter, including, but not limited to, a HI promoter, a human U6 promoter, a mouse U6 promoter, or a swine U6 promoter. A promoter will generally be one that is able to promote transcription in a mammalian cell.

A variety of promoters are known in the art, which in some aspects, can be used herein. Nonlimiting examples of promoters that can be used herein in some aspects include: human EFlα, human cytomegalovirus (CMV) (U.S. Pat. No. 5,168,062, which is incorporated herein by reference for the purposes described herein), human ubiquitin C (UBC), mouse phosphoglycerate kinase 1, polyoma adenovirus, simian virus 40 (SV40), β-globin, β-actin, α-fetoprotein, γ-globin, β-interferon, γ-glutamyl transferase, mouse mammary tumor virus (MMTV), Rous sarcoma virus, rat insulin, glyceraldehyde-3-phosphate dehydrogenase, metallothionein II (MT II), amylase, cathepsin, MI muscarinic receptor, retroviral LTR (e.g., human T-cell leukemia virus HTLV), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2K b, HSP70, proliferin, tumor necrosis factor, thyroid stimulating hormone a gene, immunoglobulin light chain, T-cell receptor, HLA DQa and DQ, interleukin-2 receptor, MHC class II, MHC class II HLA-DRa, muscle creatine kinase, prealbumin (transthyretin), elastase I, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, and Gibbon Ape Leukemia Virus (GAL V) promoters. Additional examples of promoters are known in the art. See, e.g., Lodish, Molecular Cell Biology, Freeman and Company, New York 2007, each of which is incorporated herein by reference for the purposes described herein. In some aspects, a promoter is the CMV immediate early promoter. In some aspects, the promoter is a CAG promoter and/or a CAG/CBA promoter.

The term “constitutive” promoter refers to a nucleotide sequence that, when operably linked with a nucleic acid encoding a gene (e.g., encoding a pseudouridine synthase and/or targeting element), causes RNA to be transcribed from the nucleic acid in a cell under most or all physiological conditions. Examples of constitutive promoters include, without limitation, the retroviral Rous sarcoma virus (RSV) LTR promoter, the cytomegalovirus (CMV) promoter (see, e.g., Boshart et al., Cell 41:521-530, 1985, which is incorporated herein by reference for the purposes described herein), the SV 40 promoter, the dihydrofolate reductase promoter, the beta-actin promoter, the phosphoglycerol kinase (PGK) promoter, and the EFl-alpha promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Additional examples of inducible promoters are known in the art. Examples of inducible promoters regulated by exogenously supplied compounds include the zinc-inducible sheep metallothionein (MT) promoter, the dexamethasone (Dex) inducible mouse mammary tumor virus (MMTV) promoter, the T7 polymerase promoter system (see e.g., WO 98/10088, which is incorporated herein by reference for the purposes described herein); the ecdysone insect promoter (see e.g., No et al., Proc. Natl. Acad Sci. U.S.A 93:3346-3351, 1996, which is incorporated herein by reference for the purposes described herein), the tetracycline-repressible system (see e.g., Gossen et al., Proc. Natl. Acad Sci. U.S.A 89:5547-5551, 1992, which is incorporated herein by reference for the purposes described herein), the tetracycline-inducible system (see e.g., Gossen et al., Science 268: 1766-1769, 1995, see also Harvey et al., Curr. Opin. Chem. Biol. 2:512-518, 1998, each of which is incorporated herein by reference for the purposes described herein), the RU486-inducible system (see e.g., Wang et al., Nat. Biotech. 15:239-243, 1997, and Wang et al., Gene Ther. 4:432-441, 1997, each of which is incorporated herein by reference for the purposes described herein), and the rapamycin-inducible system (see e.g., Magari et al., J Clin. Invest. 100:2865-2872, 1997, which is incorporated herein by reference for the purposes described herein).

The term “tissue-specific” promoter refers to a promoter that is active only in certain specific cell types and/or tissues (e.g., transcription of a specific gene occurs only within cells expressing transcription regulatory and/or control proteins that bind to the tissue-specific promoter). In some aspects, regulatory and/or control sequences impart tissue-specific gene expression capabilities. In some cases, tissue-specific regulatory and/or control sequences bind tissue-specific transcription factors that induce transcription in a tissue-specific manner. In some aspects, a tissue-specific promoter is a neuron-specific promoter. In some aspects, a tissue-specific promoter is hematopoietic lineage cell-specific promoter. In some aspects, a tissue-specific promoter is an immune cell-specific promoter.

### 3. Enhancers

In some aspects, a construct can include an enhancer sequence. The term “enhancer” as used herein refers to a nucleotide sequence that can increase the level of transcription of a nucleic acid encoding a protein and/or RNA molecule of interest (e.g., pseudouridine synthase and/or targeting element), and/or increase or modify the translational efficiency of a transcript following transcription. In some aspects, enhancer sequences (generally 50-1500 bp in length) generally increase the level of transcription by providing additional binding sites for transcription-associated proteins (e.g., transcription factors), and/or stabilize or modify post-transcriptional regulatory machinery. In some aspects, an enhancer sequence is found within an intronic sequence. In some aspects, an enhancer sequence is found in a 3′ and/or 5′ UTR. In some aspects, an enhancer region is found downstream of a coding sequence comprising a transgene and proximal to a poly adenylation sequence. Unlike promoter sequences, enhancer sequences can act at much larger distance away from the transcription start site (e.g., as compared to a promoter). Non-limiting examples of enhancers include a woodchuck hepatitis virus post-transcriptional regulatory element (WPRE), RSV enhancer, a CMV enhancer, and/or a SV40 enhancer.

### 4. Flanking Untranslated Regions, 5′ UTR and 3′ UTR

In some aspects, any of the constructs described herein can include an untranslated region (UTR), such as a 5′ UTR or a 3′ UTR. UTRs of a gene are transcribed but not translated. A 5′ UTR starts at the transcription start site and continues to the start codon but does not include the start codon. A 3′ UTR starts immediately following the stop codon and continues until the transcriptional termination signal. The regulatory and/or control features of a UTR can be incorporated into any of the constructs, particles, polynucleotides, compositions, kits, or methods as described herein to enhance or otherwise modulate the expression of a gene.

Natural 5′ UTRs include a sequence that plays a role in translation initiation. In some aspects, a 5′ UTR can comprise sequences, like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), and the start codon is followed by another “G”. In some aspects, 5′ UTRs also form secondary structures that are involved in elongation factor binding. In some aspects, a 5′ UTR is included in any of the constructs described herein. Non-limiting examples of 5′ UTRs, including those from the following genes: albumin, serum amyloid A, Apolipoprotein A/B/E, transferrin, alpha fetoprotein, erythropoietin, and Factor VIII, can be used to enhance expression of a nucleic acid molecule, such as an mRNA.

3′ UTRs are known to have stretches of adenosines and uridines (in the RNA form) or thymidines (in the DNA form) embedded in them. These AU-rich signatures are particularly prevalent in genes with high rates of turnover. Based on their sequence features and functional properties, the AU-rich elements (AREs) can be separated into three classes (see e.g., Chen et al., Mol. Cell. Biol. 15:5777-5788, 1995; Chen et al., Mol. Cell Biol. 15:2010-2018, 1995, each of which is incorporated herein by reference for the purposes described herein): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif, two well-studied examples of this class are c-Jun and myogenin mRNAs.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules may lead to HuR binding and thus, stabilization of the message in vivo.

In some aspects, the introduction, removal, or modification of 3′ UTR AREs can be used to modulate the stability of an mRNA encoding a gene of interest. In other aspects, AREs can be removed or mutated to increase the intracellular stability and thus increase translation and production of a protein of interest.

In some aspects, non-ARE sequences may be incorporated into the 5′ or 3′ UTRs. In some aspects, introns or portions of intron sequences may be incorporated into the flanking regions of the polynucleotides in any of the constructs, particles, polynucleotides, compositions, kits, and methods provided herein. Incorporation of intronic sequences may increase protein production as well as mRNA levels.

### 5. Internal Ribosome Entry Sites (IRES)

In some aspects, a construct described herein can include an internal ribosome entry site (IRES). An IRES forms a complex secondary structure that allows translation initiation to occur from any position with an mRNA immediately downstream from where the IRES is located (see, e.g., Pelletier and Sonenberg, Mol. Cell. Biol. 8(3): 1103-1112, 1988, which is incorporated herein by reference for the purposes described herein). There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HA V), hepatitis C virus (HCV), and poliovirus (PV) (see e.g., Alberts, Molecular Biology of the Cell, Garland Science, 2002; and Hellen et al., Genes Dev. 15(13):1593-612, 2001, each of which are incorporated herein by reference for the purposes described herein).

In some aspects, an IRES sequence that is incorporated into a construct described herein is the foot and mouth disease virus (FMDV) 2A sequence. The Foot and Mouth Disease Virus 2A sequence is a small peptide (approximately 18 amino acids in length) that has been shown to mediate the cleavage of polyproteins (see e.g., Ryan, M D et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999, each of which is incorporated herein by reference for the purposes described herein). The cleavage activity of the 2A sequence has previously been demonstrated in artificial systems including plasmids and gene therapy constructs (e.g., AAV and retroviruses) (see e.g., Ryan et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999; de Felipe et al., Gene Therapy 6: 198-208, 1999; de Felipe et al., Human Gene Therapy II: 1921-1931, 2000; and Klump et al., Gene Therapy 8:811-817, 2001, each of which is incorporated herein by reference for the purposes described herein).

In some aspects, an IRES can be utilized in an AAV construct. In some aspects, a construct can include a polynucleotide internal ribosome entry site (IRES). In some aspects, an IRES can be part of a composition comprising more than one construct. In some aspects, an IRES is used to produce more than one polypeptide from a single gene transcript.

### 6. Splice Sites

In some aspects, any of the constructs provided herein can include splice donor and/or splice acceptor sequences, which are functional during RNA processing occurring during transcription. In some aspects, splice sites are involved in trans-splicing.

### 7. Polyadenylation Sequences

In some aspects, a construct provided herein can include a polyadenylation (poly(A)) signal sequence. Most nascent eukaryotic mRNAs possess a poly(A) tail at their 3′ end, which is added during a complex process that includes cleavage of the primary transcript and a coupled polyadenylation reaction driven by the poly(A) signal sequence (see, e.g., Proudfoot et al., Cell 108:501-512, 2002, which is incorporated herein by reference for the purposes described herein). A poly(A) tail confers mRNA stability and transferability (see e.g., Molecular Biology of the Cell, Third Edition by B. Alberts et al., Garland Publishing, 1994, which is incorporated herein by reference for the purposes described herein). In some aspects, a poly(A) signal sequence is positioned 3′ to a coding sequence.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. A 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In some aspects, a poly(A) tail is added onto transcripts that contain a specific sequence, e.g., a poly(A) signal. A poly(A) tail and associated proteins aid in protecting mRNA from degradation by exonucleases. Polyadenylation also plays a role in transcription termination, export of the mRNA from the nucleus, and translation. Polyadenylation typically occurs in the nucleus immediately after transcription of DNA into RNA, but also can occur later in the cytoplasm. After transcription has been terminated, an mRNA chain is cleaved through the action of an endonuclease complex associated with RNA polymerase. A cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

There are several poly(A) signal sequences that can be used in some aspects, including those derived from bovine growth hormone (bGH) (Woychik et al., Proc. Natl. Acad Sci. U.S.A. 81(13):3944-3948, 1984; U.S. Pat. No. 5,122,458, each of which is incorporated herein by reference for the purposes described herein), mouse-β-globin, mouse-α-globin (Orkin et al., EMBO J 4(2):453-456, 1985; Thein et al., Blood 71(2):313-319, 1988, each of which is incorporated herein by reference for the purposes described herein), human collagen, polyoma virus (Batt et al., Mol. Cell Biol. 15(9):4783-4790, 1995, which is incorporated herein by reference for the purposes described herein), the Herpes simplex virus thymidine kinase gene (HSV TK), IgG heavy-chain gene polyadenylation signal (US 2006/0040354, which is incorporated herein by reference for the purposes described herein), human growth hormone (hGH) (Szymanski et al., Mol Therapy 15(7):1340-1347, 2007, which is incorporated herein by reference for the purposes described herein), and/or the group consisting of SV40 poly(A) site, such as the SV40 late and early poly(A) site (see e.g., Schek et al., Mol Cell Biol. 12(12):5386-5393, 1992, which is incorporated herein by reference for the purposes described herein).

In some aspects, the poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414, which is incorporated herein by reference for the purposes described herein). In some aspects, a poly(A) signal sequence can be a synthetic polyadenylation site (see, e.g., the pCl-neo expression construct of Promega that is based on Levitt et al., Genes Dev. 3(7):1019-1025, 1989, which is incorporated herein by reference for the purposes described herein).

### 8. Additional Sequences

In some aspects, constructs of the present disclosure may comprise a 2A element or sequence. In some aspects, constructs of the present disclosure may include one or more cloning sites. In some such aspects, cloning sites may not be fully removed prior to manufacturing for administration to a subject. In some aspects, cloning sites may have functional roles including as linker sequences, or as portions of a Kozak site. As will be appreciated by those skilled in the art, cloning sites may vary significantly in primary sequence while retaining their desired function.

In some aspects, a 2A element is a T2A, P2A, E2A, and/or F2A element. In some aspects, a 2A sequence may comprise an optional 5′ linker sequence, such as but not limited to GSG (e.g., Glycine, Serine, Glycine).

### 9. Destabilization Domains

In some aspects, any of the constructs provided herein can optionally include a sequence encoding a destabilizing domain (“a destabilizing sequence”) for temporal and/or spatial control of protein expression. Non-limiting examples of destabilizing sequences include sequences encoding a FK506 sequence, a dihydrofolate reductase (DHFR) sequence, or other exemplary destabilizing sequences.

In the absence of a stabilizing ligand, a protein sequence operatively linked to a destabilizing sequence is degraded by ubiquitination. In contrast, in the presence of a stabilizing ligand, protein degradation is inhibited, thereby allowing the protein sequence operatively linked to the destabilizing sequence to be actively expressed. As a positive control for stabilization of protein expression, protein expression can be detected by conventional means, including enzymatic, radiographic, colorimetric, fluorescence, or other spectrographic assays, fluorescent activating cell sorting (FACS) assays, and/or immunological assays (e.g., enzyme linked immunosorbent assay (ELISA), radioimmunoassay (RIA), and immunohistochemistry).

Additional examples of destabilizing sequences are known in the art. In some aspects, the destabilizing sequence is a FK506- and rapamycin-binding protein (FKBP12) sequence, and the stabilizing ligand is Shield-I (Shld1) (see e.g., Banaszynski et al. (2012) Cell 126(5):995-1004, which is incorporated herein by reference for the purposes described herein). In some aspects, a destabilizing sequence is a DI-FR sequence, and a stabilizing ligand is trimethoprim (TMP) (see e.g., Iwamoto et al., (2010) Chem Biol 17:981-988, which is incorporated herein by reference for the purposes described herein).

### 10. Reporter Sequences or Elements

In some aspects, constructs provided herein can optionally include a sequence encoding a reporter polypeptide and/or protein (“a reporter sequence”). Non-limiting examples of reporter sequences include DNA sequences encoding: a beta-lactamase, a betagalactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences are known in the art. When associated with control elements which drive their expression, the reporter sequence can provide signals detectable by conventional means, including enzymatic, radiographic, colorimetric, fluorescence, or other spectrographic assays, fluorescent activating cell sorting (FACS) assays and/or immunological assays (e.g., enzyme linked immunosorbent assay (ELISA), radioimmunoassay (RIA), and immunohistochemistry). In some aspects, a reporter sequence is a FLAG tag (e.g., a 3×FLAG tag), and the presence of a construct carrying the FLAG tag in a cell is detected by protein binding or detection assays (e.g., Western blots, immunohistochemistry, radioimmunoassay (RIA), mass spectrometry).

In some aspects, a reporter sequence is the Lacz gene, and the presence of a construct carrying the Lacz gene in a cell is detected by assays for beta-galactosidase activity. In some aspects, a reporter sequence is a fluorescent protein (e.g., green fluorescent protein (GFP)) or luciferase. In aspects where a reporter sequence is a fluorescent protein or luciferase, the presence of a construct carrying the fluorescent protein or luciferase in a cell may be measured by fluorescent imaging techniques (e.g., fluorescent microscopy or FACS) or light production in a luminometer (e.g., a spectrophotometer or an IVIS imaging instrument). In some aspects, a reporter sequence can be used to verify tissue-specific targeting capabilities and/or tissue-specific promoter regulatory and/or control activity of any of the constructs described herein.

### III. Methods of Use

In some aspects, provided herein are methods of using the provided compositions and technologies disclosed herein.

In certain aspects, provided herein are methods of treating diseases and/or disorders associated with (e.g., correlated with and/or potentially caused by) insufficient functional protein levels. For example, in some aspects a disease and/or disorder is associated with and/or characterized by gene haploinsufficiency, gene downregulation, and/or gene translation inhibiting polymorphisms (e.g., premature stop codons, rare codons, missense codons, etc.), etc. In some aspects, a diseases and/or disorder is associated with a premature stop codon in a gene. In some aspects, a disease and/or disorder is associated with haploinsufficiency. In some aspects, a disease and/or disorder is associated with genes comprising one or more rare codons. In some aspects, a rare codon comprises, consists essentially of, or consists of one or more of ATA, TTT, CAT, TTA, AAT, or TAT (AUA, UUU, CAU, UUA, AAU, or UAU in RNA, respectively). In some aspects, a rare codon is ATA (AUA in RNA). In some aspects, a disease and/or disorder is associated with genes comprising more than one rare codon. In some aspects, a disease and/or disorder is associated with genes comprising more than one of the same rare codon.

In some aspects, a disease and/or disorder is associated with a protein and/or polypeptide that has a relatively small size. In some aspects, a relatively small protein is one that is less than or equal to 500, 450, 400, 350, 300, 250, 225, 200, 195, 190, 185, 180, 175, 170, 165, 160, 155, 150, 145, 140, 135, 130, 125, 120, 115, 110, 105, 100, 95, 90, 85, 80, 75, 70, 65, 60, 55, 50, 45, 40, 35, 30, 25, 20, 15, 10, or 5 amino acids, or less than 5 amino acids, or any range derivable therein. In some aspects, a relatively small protein and/or polypeptide is one that is less than or equal to 50, 45, 40, 35, 30, 25, 22.5, 20.0, 19.5, 19.0, 18.5, 18.0, 17.5, 17.0, 16.5, 16.0, 15.5, 15.0, 14.5, 14.0, 13.5, 13.0, 12.5, 12.0, 11.5, 11.0, 10.5, 10.0, 9.5, 9.0, 8.5, 8.0, 7.5, 7.0, 6.5, 6.0, 5.5, 5.0, 4.5, 4.0, 3.5, 3.0, 2.5, 2.0, 1.5, or 1 kDa, or less than 1 kDa, or any range derivable therein.

In some aspects, a disease and/or disorder is associated with a relatively small protein and one or more rare codons (e.g., one or more ATA (AUA in RNA), TTT (UUU in RNA), etc).

In some aspects, a disease and/or disorder is associated with insufficient levels of Glomulin (GLMN, FKBP Associated Protein) protein. In some aspects, a disease and/or disorder is associated with insufficient levels of Protection of Telomers 1 (POT1) protein. In some aspects, a disease and/or disorder is associated with insufficient levels of TANK-binding kinase 1 (TBK1) protein. In some aspects, a disease and/or disorder is associated with insufficient levels of Krev1 interaction trapped gene 1 (KRIT1). In some aspects, a disease and/or disorder is associated with insufficient levels of Nibrin (NBN) protein. In some aspects, a disease and/or disorder is associated with insufficient levels of FA Complementation Group B (FANCB) protein. In some aspects, a disease and/or disorder is associated with insufficient levels of Desmocollin 2 (DSC2) protein. In some aspects, a disease and/or disorder is associated with insufficient levels of DNA Ligase 4 (LIG4) protein. In some aspects, a disease and/or disorder is associated with insufficient levels of ATPase Secretory Pathway Ca2+ Transporting 1 (ATP2C1) protein. In some aspects, a disease and/or disorder is associated with insufficient levels of Post-GPI Attachment To Proteins Inositol Deacylase 1 (PGAP1) protein. In some aspects, a disease and/or disorder is associated with insufficient levels of RB Transcriptional Corepressor 1 (RB1) protein. In some aspects, a disease and/or disorder is associated with insufficient levels of MutS Homolog 2 (MSH2) protein. In some aspects, a disease and/or disorder is associated with insufficient levels of RAS P21 Protein Activator 1 (RASA1) protein. In some aspects, a disease and/or disorder is associated with insufficient levels of Desmoglein 1 (DSG1) protein. In some aspects, a disease and/or disorder is associated with insufficient levels of Kinesin Family Member 11 (KIF11) protein. In some aspects, a disease and/or disorder is associated with insufficient levels of Cell Adhesion Molecule L1 Like (CHL1) protein. In some aspects, a disease and/or disorder is associated with insufficient levels of RAD50 Double Strand Break Repair Protein (RAD50) protein. In some aspects, a disease and/or disorder is associated with insufficient levels of ATPase Copper Transporting Alpha (ATP7A) protein. In some aspects, a disease and/or disorder is associated with insufficient levels of Pleckstrin Homology Domain Interacting Protein (PHIP) protein. In some aspects, a disease and/or disorder is associated with insufficient levels of Sucrase-Isomaltase (SI) protein. In some aspects, a disease and/or disorder is associated with insufficient levels of APC Regulator Of WNT Signaling Pathway (APC) protein. In some aspects, a disease and/or disorder is associated with insufficient levels of ATM Serine/Threonine Kinase (ATM) protein. In some aspects, a disease and/or disorder is associated with insufficient levels of Breast Cancer Gene 2 (BRCA2) protein. In some aspects, a disease and/or disorder is associated with insufficient levels of KRAS Proto-Oncogene, GTPase (KRAS) protein. In some aspects, a disease and/or disorder is associated with insufficient levels of CF Transmembrane Conductance Regulator (CTFR) protein. In some aspects, a disease and/or disorder is associated with insufficient levels of Tumor Protein P53 (p53) protein. In some aspects, a disease and/or disorder is associated with insufficient levels of Cell Division Cycle 6 (CDC6) protein. In some aspects, a disease and/or disorder is associated with insufficient levels of Autocrine Motility Factor Receptor (AMFR) protein. In some aspects, a disease and/or disorder is associated with insufficient levels of Sterol Carrier Protein 2 (SCP2) protein. In some aspects, a disease and/or disorder is associated with insufficient levels of ERH MRNA Splicing And Mitosis Factor (ERH) protein. In some aspects, a disease and/or disorder is associated with insufficient levels of CF Transmembrane Conductance Regulator (CFTR) protein.

### A. Target RNA

In some aspects, methods provided herein comprise site-specific targeting and/or modification of one or more target RNA molecules. In some aspects, a target RNA is a messenger RNA (mRNA). In some aspects, a target RNA is not a ribosomal RNA (rRNA). In some aspects, a target RNA is not a tRNA. In some aspects, a target RNA is not a non-coding RNA, such as miRNA, lncRNA, piRNA, siRNA, eRNA, or promoter-associated RNA.

In some aspects, a target site in a target RNA is a premature stop codon. In some aspects, a target site in a target RNA is not a premature stop codon. In some aspects, a target site in a target RNA is a non-canonical codon and/or a codon that is a result of a mutation. In some aspects, a target site is a codon containing 1, 2, or 3 uracils.

In some aspects, a target site in a target RNA comprises one or more rare codons. In some aspects, a rare codon is ATA (AUA in RNA), TTT (UUU in RNA), CAT (CAU in RNA), TTA (UUA in RNA), AAT (AAU in RNA), or TAT (UAU in RNA). In some aspects, a rare codon is ATA (AUA in RNA). In some aspects, a rare codon can be enriched in multiple different mammalian tissues. In some aspects, a rare codon can be enriched in specific mammalian tissues. In some aspects, a rare codon can be enriched in specific cell types.

In some aspects, a target RNA is associated with a disease characterized by haploinsufficiency. In some aspects, a target RNA encodes a relatively small protein. In some aspects, a target RNA comprises one or more rare codons (e.g., one or more ATA, TTT, etc.). In some aspects, a target RNA encodes a relatively small protein and comprises one or more rare codons (e.g., one or more ATA, TTT, etc.). In some aspects, a target RNA comprises more than one rare codon.

In some aspects, a target gene is GLMN(ENSG00000174842.17). In some aspects, a target RNA is GLMNtranscript ENST00000370360.8. In some aspects, a target GLMNRNA comprises one or more rare codons. In some aspects, a target GLMN RNA comprises one or more ATA (AUA in RNA) codons.

In some aspects, a target gene is POT1 (ENSG00000128513.16). In some aspects, a target RNA is POT1transcript ENST00000357628.8. In some aspects, a target POT1 RNA comprises one or more rare codons. In some aspects, a target POT1 RNA comprises one or more ATA (AUA in RNA) codons.

In some aspects, a target gene is TBK1(ENSG00000183735.11). In some aspects, a target RNA is TBK1transcript ENST00000331710.10. In some aspects, a target TBK1 RNA comprises one or more rare codons. In some aspects, a target TBK1 RNA comprises one or more ATA (AUA in RNA) codons.

In some aspects, a target gene is KRIT1(ENSG00000001631.17). In some aspects, a target RNA is KRIT1transcript ENST00000394505.7. In some aspects, a target KRIT1 RNA comprises one or more rare codons. In some aspects, a target KRIT1 RNA comprises one or more ATA (AUA in RNA) codons.

In some aspects, a target gene is NBN(ENSG00000104320.14). In some aspects, a target RNA is NBN transcript ENST00000265433.8. In some aspects, a target NBN RNA comprises one or more rare codons. In some aspects, a target NBNRNA comprises one or more ATA (AUA in RNA) codons.

In some aspects, a target gene is FANCB (ENSG00000181544.15). In some aspects, a target RNA is FANCB transcript ENST00000650831.1. In some aspects, a target FANCB RNA comprises one or more rare codons. In some aspects, a target FANCB RNA comprises one or more ATA (AUA in RNA) codons.

In some aspects, a target gene is DSC2 (ENSG00000134755.18). In some aspects, a target RNA is DSC2 transcript ENST00000280904.11. In some aspects, a target DSC2 RNA comprises one or more rare codons. In some aspects, a target DSC2 RNA comprises one or more ATA (AUA in RNA) codons.

In some aspects, a target gene is LIG4 (ENSG00000174405.15). In some aspects, a target RNA is LIG4 transcript ENST00000442234.6. In some aspects, a target LIG4 RNA comprises one or more rare codons. In some aspects, a target LIG4 RNA comprises one or more ATA (AUA in RNA) codons.

In some aspects, a target gene is ATP2C1 (ENSG00000017260.20). In some aspects, a target RNA is ATP2C1 transcript ENST00000510168.6. In some aspects, a target ATP2C1 RNA comprises one or more rare codons. In some aspects, a target ATP2C1 RNA comprises one or more ATA (AUA in RNA) codons.

In some aspects, a target gene is PGAP1 (ENSG00000197121.15). In some aspects, a target RNA is PGAP1 transcript ENST00000354764.9. In some aspects, a target PGAP1 RNA comprises one or more rare codons. In some aspects, a target PGAP1 RNA comprises one or more ATA (AUA in RNA) codons.

In some aspects, a target gene is RB1(ENSG00000139687.16). In some aspects, a target RNA is RB1 transcript ENST00000267163.6. In some aspects, a target RB1 RNA comprises one or more rare codons. In some aspects, a target RB1 RNA comprises one or more ATA (AUA in RNA) codons.

In some aspects, a target gene is MSH2 (ENSG00000095002.15). In some aspects, a target RNA is MSH2 transcript ENST00000233146.7. In some aspects, a target MSH2 RNA comprises one or more rare codons. In some aspects, a target MSH2 RNA comprises one or more ATA (AUA in RNA) codons.

In some aspects, a target gene is RASA1 (ENSG00000145715.15). In some aspects, a target RNA is RASA1 transcript ENST00000274376.11. In some aspects, a target RASA1 RNA comprises one or more rare codons. In some aspects, a target RASA1 RNA comprises one or more ATA (AUA in RNA) codons.

In some aspects, a target gene is DSG1 (ENSG00000134760.6). In some aspects, a target RNA is DSG1 transcript ENST00000257192.5. In some aspects, a target DSG1 RNA comprises one or more rare codons. In some aspects, a target DSG1 RNA comprises one or more ATA (AUA in RNA) codons.

In some aspects, a target gene is KIF11 (ENSG00000138160.7). In some aspects, a target RNA is KIF11 transcript ENST00000260731.5. In some aspects, a target KIF11 RNA comprises one or more rare codons. In some aspects, a target KIF11 RNA comprises one or more ATA (AUA in RNA) codons.

In some aspects, a target gene is CHL1 (ENSG00000134121.10). In some aspects, a target RNA is CHL1 transcript ENST00000256509.7. In some aspects, a target CHL1 RNA comprises one or more rare codons. In some aspects, a target CHL1 RNA comprises one or more ATA (AUA in RNA) codons.

In some aspects, a target gene is RAD50 (ENSG00000113522.14). In some aspects, a target RNA is RAD50 transcript ENST00000378823.8. In some aspects, a target RAD50 RNA comprises one or more rare codons. In some aspects, a target RAD50 RNA comprises one or more ATA (AUA in RNA) codons.

In some aspects, a target gene is ATP7A (ENSG00000165240.22). In some aspects, a target RNA is ATP7A transcript ENST00000341514.11. In some aspects, a target ATP7A RNA comprises one or more rare codons. In some aspects, a target ATP7A RNA comprises one or more ATA (AUA in RNA) codons.

In some aspects, a target gene is PHIP (ENSG00000146247.14). In some aspects, a target RNA is PHIP transcript ENST00000275034.5. In some aspects, a target PHIP RNA comprises one or more rare codons. In some aspects, a target PHIP RNA comprises one or more ATA (AUA in RNA) codons.

In some aspects, a target gene is SI (ENSG00000090402.8). In some aspects, a target RNA is SI transcript ENST00000264382.8. In some aspects, a target SIRNA comprises one or more rare codons. In some aspects, a target SIRNA comprises one or more ATA (AUA in RNA) codons.

In some aspects, a target gene is APC (ENSG00000134982.17). In some aspects, a target RNA is APC transcript ENST00000257430.9. In some aspects, a target APC RNA comprises one or more rare codons. In some aspects, a target APC RNA comprises one or more ATA (AUA in RNA) codons.

In some aspects, a target gene is ATM(ENSG00000149311.20). In some aspects, a target RNA is ATM transcript ENST00000675843.1. In some aspects, a target ATM RNA comprises one or more rare codons. In some aspects, a target ATM RNA comprises one or more ATA (AUA in RNA) codons.

In some aspects, a target gene is BRCA2 (ENSG00000139618.17). In some aspects, a target RNA is BRCA2 transcript ENST00000380152.8. In some aspects, a target BRCA2 RNA comprises one or more rare codons. In some aspects, a target BRCA2 RNA comprises one or more ATA (AUA in RNA) codons.

In some aspects, a target gene is DMD (ENSG00000198947). In some aspects, a DMD RNA comprises one or more rare codons.

In some aspects, a target gene is HBB (ENSG00000244734). In some aspects, a HBB RNA comprises one or more rare codons.

In some aspects, a target gene is IDUA (ENSG00000127415). In some aspects, a IDUA RNA comprises one or more rare codons.

In some aspects, a target gene is NF1(ENSG00000196712). In some aspects, a NF RNA comprises one or more rare codons.

In some aspects, a target gene and/or transcript thereof comprises KRAS, p53, DMD, HBB, IDUA NF1, CDC6, AMFR, SCP2, ERH, CFTR, GLMN, POT1, TBK1, KRIT1, NBN, FANCB, DSC2, LIG4, ATP2C1, PGAP1, RB1, MSH2, RASA1, DSG1, KIF11, CHL1, RAD50, ATP7A, PHIP, SI, APC, ATM, and/or BRCA2.

In certain aspects, pseudouridine installation in a target RNA does not necessarily result in increased RNA stability. In certain aspects, pseudouridine installation in a target RNA results in increased RNA stability. In certain aspects, pseudouridine installation in a target mRNA increases translational efficiencies, translational readthrough, and/or translational rates.

SEQ ID NO: 29-WT p53 isoform 1 polynucleotide sequence
(SEQ ID NO: 29)
ATGGAGGAGCCGCAGTCAGATCCTAGCGTCGAGCCCCCTCTGAGTCAGGAAACATTTTCAGA
CCTATGGAAACTACTTCCTGAAAACAACGTTCTGTCCCCCTTGCCGTCCCAAGCAATGGATG
ATTTGATGCTGTCCCCGGACGATATTGAACAATGGTTCACTGAAGACCCAGGTCCAGATGAA
GCTCCCAGAATGCCAGAGGCTGCTCCCCCCGTGGCCCCTGCACCAGCAGCTCCTACACCGGC
GGCCCCTGCACCAGCCCCCTCCTGGCCCCTGTCATCTTCTGTCCCTTCCCAGAAAACCTACC
AGGGCAGCTACGGTTTCCGTCTGGGCTTCTTGCATTCTGGGACAGCCAAGTCTGTGACTTGC
ACGTACTCCCCTGCCCTCAACAAGATGTTTTGCCAACTGGCCAAGACCTGCCCTGTGCAGCT
GTGGGTTGATTCCACACCCCCGCCCGGCACCCGCGTCCGCGCCATGGCCATCTACAAGCAGT
CACAGCACATGACGGAGGTTGTGAGGCGCTGCCCCCACCATGAGCGCTGCTCAGATAGCGAT
GGTCTGGCCCCTCCTCAGCATCTTATCCGAGTGGAAGGAAATTTGCGTGTGGAGTATTTGGA
TGACAGAAACACTTTTCGACATAGTGTGGTGGTGCCCTATGAGCCGCCTGAGGTTGGCTCTG
ACTGTACCACCATCCACTACAACTACATGTGTAACAGTTCCTGCATGGGCGGCATGAACCGG
AGGCCCATCCTCACCATCATCACACTGGAAGACTCCAGTGGTAATCTACTGGGACGGAACAG
CTTTGAGGTGCGTGTTTGTGCCTGTCCTGGGAGAGACCGGCGCACAGAGGAAGAGAATCTCC
GCAAGAAAGGGGAGCCTCACCACGAGCTGCCCCCAGGGAGCACTAAGCGAGCACTGCCCAAC
AACACCAGCTCCTCTCCCCAGCCAAAGAAGAAACCACTGGATGGAGAATATTTCACCCTTCA
GATCCGTGGGCGTGAGCGCTTCGAGATGTTCCGAGAGCTGAATGAGGCCTTGGAACTCAAGG
ATGCCCAGGCTGGGAAGGAGCCAGGGGGGAGCAGGGCTCACTCCAGCCACCTGAAGTCCAAA
AAGGGTCAGTCTACCTCCCGCCATAAAAAACTCATGTTCAAGACAGAAGGGCCTGACTCAGA
C
SEQ ID NO: 30-R213X p53 isoform 1 polynucleotide sequence
(SEQ ID NO: 30)
ATGGAGGAGCCGCAGTCAGATCCTAGCGTCGAGCCCCCTCTGAGTCAGGAAACATTTTCAGA
CCTATGGAAACTACTTCCTGAAAACAACGTTCTGTCCCCCTTGCCGTCCCAAGCAATGGATG
ATTTGATGCTGTCCCCGGACGATATTGAACAATGGTTCACTGAAGACCCAGGTCCAGATGAA
GCTCCCAGAATGCCAGAGGCTGCTCCCCCCGTGGCCCCTGCACCAGCAGCTCCTACACCGGC
GGCCCCTGCACCAGCCCCCTCCTGGCCCCTGTCATCTTCTGTCCCTTCCCAGAAAACCTACC
AGGGCAGCTACGGTTTCCGTCTGGGCTTCTTGCATTCTGGGACAGCCAAGTCTGTGACTTGC
ACGTACTCCCCTGCCCTCAACAAGATGTTTTGCCAACTGGCCAAGACCTGCCCTGTGCAGCT
GTGGGTTGATTCCACACCCCCGCCCGGCACCCGCGTCCGCGCCATGGCCATCTACAAGCAGT
CACAGCACATGACGGAGGTTGTGAGGCGCTGCCCCCACCATGAGCGCTGCTCAGATAGCGAT
GGTCTGGCCCCTCCTCAGCATCTTATCCGAGTGGAAGGAAATTTGCGTGTGGAGTATTTGGA
TGACAGAAACACTTTTTGACATAGTGTGGTGGTGCCCTATGAGCCGCCTGAGGTTGGCTCTG
ACTGTACCACCATCCACTACAACTACATGTGTAACAGTTCCTGCATGGGCGGCATGAACCGG
AGGCCCATCCTCACCATCATCACACTGGAAGACTCCAGTGGTAATCTACTGGGACGGAACAG
CTTTGAGGTGCGTGTTTGTGCCTGTCCTGGGAGAGACCGGCGCACAGAGGAAGAGAATCTCC
GCAAGAAAGGGGAGCCTCACCACGAGCTGCCCCCAGGGAGCACTAAGCGAGCACTGCCCAAC
AACACCAGCTCCTCTCCCCAGCCAAAGAAGAAACCACTGGATGGAGAATATTTCACCCTTCA
GATCCGTGGGCGTGAGCGCTTCGAGATGTTCCGAGAGCTGAATGAGGCCTTGGAACTCAAGG
ATGCCCAGGCTGGGAAGGAGCCAGGGGGGAGCAGGGCTCACTCCAGCCACCTGAAGTCCAAA
AAGGGTCAGTCTACCTCCCGCCATAAAAAACTCATGTTCAAGACAGAAGGGCCTGACTCAGA
C
SEQ ID NO: 31-KRAS transcript variant “a” coding polynucleotide sequence (the ATA
associated with each of U62, U107, U251, and U563 are underlined)
(SEQ ID NO: 31)
ATGACTGAATATAAACTTGTGGTAGTTGGAGCTGGTGGCGTAGGCAAGAGTGCCTTGACGAT
ACAGCTAATTCAGAATCATTTTGTGGACGAATATGATCCAACAATAGAGGATTCCTACAGGA
AGCAAGTAGTAATTGATGGAGAAACCTGTCTCTTGGATATTCTCGACACAGCAGGTCAAGAG
GAGTACAGTGCAATGAGGGACCAGTACATGAGGACTGGGGAGGGCTTTCTTTGTGTATTTGC
CATAAATAATACTAAATCATTTGAAGATATTCACCATTATAGAGAACAAATTAAAAGAGTTA
AGGACTCTGAAGATGTACCTATGGTCCTAGTAGGAAATAAATGTGATTTGCCTTCTAGAACA
GTAGACACAAAACAGGCTCAGGACTTAGCAAGAAGTTATGGAATTCCTTTTATTGAAACATC
AGCAAAGACAAGACAGAGAGTGGAGGATGCTTTTTATACATTGGTGAGAGAGATCCGACAAT
ACAGATTGAAAAAAATCAGCAAAGAAGAAAAGACTCCTGGCTGTGTGAAAATTAAAAAATGC
ATTATAATGTAA
SEQ ID NO: 32-KRAS transcript variant “b” coding polynucleotide sequence (the ATA
associated with each of U62, U107, and U251 are underlined)
(SEQ ID NO: 32)
ATGACTGAATATAAACTTGTGGTAGTTGGAGCTGGTGGCGTAGGCAAGAGTGCCTTGACGAT
ACAGCTAATTCAGAATCATTTTGTGGACGAATATGATCCAACAATAGAGGATTCCTACAGGA
AGCAAGTAGTAATTGATGGAGAAACCTGTCTCTTGGATATTCTCGACACAGCAGGTCAAGAG
GAGTACAGTGCAATGAGGGACCAGTACATGAGGACTGGGGAGGGCTTTCTTTGTGTATTTGC
CATAAATAATACTAAATCATTTGAAGATATTCACCATTATAGAGAACAAATTAAAAGAGTTA
AGGACTCTGAAGATGTACCTATGGTCCTAGTAGGAAATAAATGTGATTTGCCTTCTAGAACA
GTAGACACAAAACAGGCTCAGGACTTAGCAAGAAGTTATGGAATTCCTTTTATTGAAACATC
AGCAAAGACAAGACAGGGTGTTGATGATGCCTTCTATACATTAGTTCGAGAAATTCGAAAAC
ATAAAGAAAAGATGAGCAAAGATGGTAAAAAGAAGAAAAAGAAGTCAAAGACAAAGTGTGTA
ATTATGTAA
SEQ ID NO: 33-KRAS transcript variant “c” coding polynucleotide sequence (the ATA
associated with each of U62, U107, U251, and U563 are underlined)
(SEQ ID NO: 33)
ATGACTGAATATAAACTTGTGGTAGTTGGAGCTGGTGGCGTAGGCAAGAGTGCCTTGACGAT
ACAGCTAATTCAGAATCATTTTGTGGACGAATATGATCCAACAATAGAGGATTCCTACAGGA
AGCAAGTAGTAATTGATGGAGAAACCTGTCTCTTGGATATTCTCGACACAGCAGGTCAAGAG
GAGTACAGTGCAATGAGGGACCAGTACATGAGGACTGGGGAGGGCTTTCTTTGTGTATTTGC
CATAAATAATACTAAATCATTTGAAGATATTCACCATTATAGAGAACAAATTAAAAGAGTTA
AGGACTCTGAAGATGTACCTATGGTCCTAGTAGGAAATAAATGTGATTTGCCTTCTAGAACA
GTAGACACAAAACAGGCTCAGGACTTAGCAAGAAGTTATGGAATTCCTTTTATTGAAACATC
AGCAAAGACAAGACAGAGAGTGGAGGATGCTTTTTATACATTGGTGAGAGAGATCCGACAAT
ACAGATTGAAAAAAATCAGCAAAGAAGAAAAGACTCCTGGCTGTGTGAAAATTAAAAAATGC
ATTATAATGTAA
SEQ ID NO: 34-KRAS transcript variant “d” coding polynucleotide sequence (the ATA
associated with each of U62, U107, and U251 are underlined)
(SEQ ID NO: 34)
ATGACTGAATATAAACTTGTGGTAGTTGGAGCTGGTGGCGTAGGCAAGAGTGCCTTGACGAT
ACAGCTAATTCAGAATCATTTTGTGGACGAATATGATCCAACAATAGAGGATTCCTACAGGA
AGCAAGTAGTAATTGATGGAGAAACCTGTCTCTTGGATATTCTCGACACAGCAGGTCAAGAG
GAGTACAGTGCAATGAGGGACCAGTACATGAGGACTGGGGAGGGCTTTCTTTGTGTATTTGC
CATAAATAATACTAAATCATTTGAAGATATTCACCATTATAGAGAACAAATTAAAAGAGTTA
AGGACTCTGAAGATGTACCTATGGTCCTAGTAGGAAATAAATGTGATTTGCCTTCTAGAACA
GTAGACACAAAACAGGCTCAGGACTTAGCAAGAAGTTATGGAATTCCTTTTATTGAAACATC
AGCAAAGACAAGACAGGGTGTTGATGATGCCTTCTATACATTAGTTCGAGAAATTCGAAAAC
ATAAAGAAAAGATGAGCAAAGATGGTAAAAAGAAGAAAAAGAAGTCAAAGACAAAGTGTGTA
ATTATGTAA

### B. Methods of Treatment

The present disclosure additionally provides methods of detecting, methods of measuring, methods of diagnosing, and/or methods of ameliorating and/or treating diseases. In some aspects, constructs, vectors, particles, polypeptides, polynucleotides, and/or compositions described herein may be comprised in a formulation with one or more additional therapeutic agents. In some aspects, constructs, vectors, particles, polypeptides, polynucleotides, and/or compositions described herein may be comprised in a formulation wherein the formulation comprises pharmaceutically acceptable excipients.

In some aspects, constructs, vectors, particles, polypeptides, polynucleotides, and/or compositions described herein may be administered to a cell in an in vitro environment. In some aspects, a cell may be derived from a subject. In some aspects, a cell is an immune cell, a stem cell, an induced pluripotent stem cell, a precursor cell, and/or a terminally differentiated cell. In some aspects, constructs, vectors, particles, polypeptides, polynucleotides, and/or compositions described herein may be administered to a cell in vivo via administration to a subject.

In some aspects, constructs, vectors, particles, polypeptides, polynucleotides, and/or compositions described herein are administered to a subject in need thereof. In some aspects, a subject may have, may be diagnosed with, or may be susceptible to a disease, such as an infectious disease, a genetic disorder, an autoimmune disease, and/or cancer.

In some aspects, a subject is a mammal. In some aspects, a subject is a domestic animal. In some aspects, a subject is a farm animal. In some aspects, a subject is a zoo animal. In some aspects, a subject is a dog or a cat. In some aspects, a subject is a cow, a horse, a sheep, or a goat. In some aspects, a subject can be but is not limited to, a dog, cat, ferret, rabbit, cow, duck, pig, goat, chicken, horse, llama, camel, ostrich, deer, turkey, dove, sheep, goose, oxen, and/or reindeer. In some aspects, a subject is a human. In some aspects, a subject is equal to, less than, or greater than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years of age.

In some aspects, administration regimens comprising constructs, vectors, particles, polypeptides, polynucleotides, and/or compositions described herein comprise administering of more than one composition, such as 2 compositions, 3 compositions, 4 compositions, or more than 4 compositions. Various combinations of the agents may be employed. In some aspects, constructs, vectors, particles, polypeptides, polynucleotides, and/or compositions of the disclosure may be administered by the same route of administration or by different routes of administration. In some aspects, agents described herein and/or additional therapeutic agents are administered intravenously, intramuscularly, subcutaneously, topically, orally, transdermally, intraperitoneally, intraorbitally, by implantation, by inhalation, intrathecally, intraventricularly, or intranasally. In some aspects, constructs, vectors, particles, polypeptides, polynucleotides, and/or compositions described herein and/or additional therapeutic agents are administered intravenously, intramuscularly, subcutaneously, topically, orally, transdermally, intraperitoneally, intraorbitally, by implantation, by inhalation, intrathecally, intraventricularly, or intranasally. In some aspects, an appropriate dosage may be determined based on the type of disease to be treated and/or prevented, severity, and/or course of the disease, the clinical condition of the individual, the individual's clinical history and response to the treatment, and/or at the discretion of the attending physician.

In some aspects, administration to a subject may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

In some aspects, the quantity to be administered, both according to number of treatments and unit dose, depends on the treatment effect desired. An effective dose is understood to refer to an amount necessary to achieve a particular effect. In the practice in certain aspects, it is contemplated that doses in the range from 0.10 mg/kg to 200 mg/kg can affect the functionality of the described agents. In certain aspects, it is contemplated that doses may comprise a composition comprising an AAV particle in a concentration of about 10⁸ to about 10¹⁴ viral genomes per ml. Furthermore, such doses can be administered at multiple times during a day, and/or on multiple days, weeks, or months.

In certain aspects, precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, 12 week, or more than 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

In some aspects, wherein a composition is proteinaceous, the proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

In some aspects, a pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some aspects, sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

In some aspects, upon formulation, compositions described herein may be administered in a manner compatible with the dosage formulation and in such amount as is therapeutically or prophylactically effective. In some aspects, formulations are administered in a variety of dosage forms, such as the type of injectable solutions described above.

### 1. Diseases or Disorders

In some aspects, constructs, vectors, particles, polypeptides, polynucleotides, and/or compositions described herein may be used in a method of preventing, treating, reducing the progression of, and/or reducing the risk of a disease or disorder. In some aspects, constructs, vectors, particles, polypeptides, polynucleotides, and/or compositions described herein may be used in treating a disease or disorder, wherein the disease or disorder is a neurodegenerative disease, an inflammatory disease, an autoimmune disease, a metabolic syndrome, a cancer, a vascular disease, a fibrotic disease, a viral infection, a bacterial infection, a fungal infection, a parasitic infection, a musculoskeletal disease (such as a myopathy), an ocular disease, or a genetic disorder.

In some aspects, the disease or disorder is an inflammatory disease. In some aspects, the inflammatory disease is arthritis, psoriatic arthritis, psoriasis, juvenile idiopathic arthritis, asthma, allergic asthma, bronchial asthma, tuberculosis, chronic airway disorder, cystic fibrosis, glomerulonephritis, membranous nephropathy, sarcoidosis, vasculitis, ichthyosis, transplant rejection, interstitial cystitis, atopic dermatitis, or inflammatory bowel disease. In some aspects, the inflammatory bowel disease is Crohn’ disease, ulcerative colitis, inflammatory bowel disease, or celiac disease.

In some aspects, the disease or disorder is an autoimmune disease. In some aspects, the autoimmune disease is systemic lupus erythematosus, type 1 diabetes, multiple sclerosis, psoriasis/psoriatic arthritis, inflammatory bowel disease, Addison's disease, Graves' disease, Sjogren's syndrome, Hashimoto's thyroiditis, Myasthenia gravis, autoimmune vasculitis, pernicious anemia, celiac disease, or rheumatoid arthritis.

In some aspects, the disease or disorder is a metabolic syndrome. In some aspects, the metabolic syndrome is acute pancreatitis, chronic pancreatitis, alcoholic liver steatosis, obesity, glucose intolerance, insulin resistance, hyperglycemia, fatty liver, dyslipidemia, hyperlipidemia, hyperhomocysteinemia, or type 2 diabetes. In some aspects, the metabolic syndrome is alcoholic liver steatosis, obesity, glucose intolerance, insulin resistance, hyperglycemia, fatty liver, dyslipidemia, hyperlipidemia, hyperhomocysteinemia, or type 2 diabetes.

In some aspects, the disease or disorder is a cancer. In some aspects, the cancer is pancreatic cancer, breast cancer, kidney cancer, bladder cancer, prostate cancer, testicular cancer, urothelial cancer, endometrial cancer, ovarian cancer, cervical cancer, renal cancer, esophageal cancer, gastrointestinal stromal tumor (GIST), multiple myeloma, cancer of secretory cells, thyroid cancer, gastrointestinal carcinoma, chronic myeloid leukemia, hepatocellular carcinoma, colon cancer, melanoma, malignant glioma, glioblastoma, glioblastoma multiforme, astrocytoma, dysplastic gangliocytoma of the cerebellum, Ewing's sarcoma, rhabdomyosarcoma, ependymoma, medulloblastoma, ductal adenocarcinoma, adenosquamous carcinoma, nephroblastoma, acinar cell carcinoma, neuroblastoma, or lung cancer. In some aspects, the cancer of secretory cells is non-Hodgkin's lymphoma, Burkitt's lymphoma, chronic lymphocytic leukemia, monoclonal gammopathy of undetermined significance (MGUS), plasmacytoma, lymphoplasmacytic lymphoma or acute lymphoblastic leukemia.

In some aspects, the disease or disorder is a musculoskeletal disease (such as a myopathy). In some aspects, the musculoskeletal disease is a myopathy, a muscular dystrophy, a muscular atrophy, a muscular wasting, or sarcopenia. In some aspects, the muscular dystrophy is Duchenne muscular dystrophy (DMD), Becker's disease, myotonic dystrophy, X-linked dilated cardiomyopathy, spinal muscular atrophy (SMA), or metaphyseal chondrodysplasia, Schmid type (MCDS). In some aspects, the myopathy is a skeletal muscle atrophy. In some aspects, the musculoskeletal disease (such as the skeletal muscle atrophy) is triggered by ageing, chronic diseases, stroke, malnutrition, bedrest, orthopedic injury, bone fracture, cachexia, starvation, heart failure, obstructive lung disease, renal failure, Acquired Immunodeficiency Syndrome (AIDS), sepsis, an immune disorder, a cancer, ALS, a burn injury, denervation, diabetes, muscle disuse, limb immobilization, mechanical unload, myositis, or a dystrophy.

In some aspects, the disease or disorder is a musculoskeletal disease. In some aspects, skeletal muscle mass, quality and/or strength are increased. In some aspects, synthesis of muscle proteins is increased. In some aspects, skeletal muscle fiber atrophy is inhibited.

In some aspects, the disease or disorder is a vascular disease. In some aspects, the vascular disease is atherosclerosis, abdominal aortic aneurism, carotid artery disease, deep vein thrombosis, Buerger's disease, chronic venous hypertension, vascular calcification, telangiectasia or lymphoedema.

In some aspects, the disease or disorder is genetic disorder. In some aspects, a genetic disorder is arrhythmogenic right ventricular dysplasia/cardiomyopathy, Brugada Syndrome, Charcot-Marie-Tooth Disease, Cleft Lip and Palate, Cleidocranial Dysplasia, Cystic Fibrosis, Familial Adenomatous Polyposis, Hirschsprungs Disease, Huntington's Disease, Klinefelter Syndrome, Kneist Syndrome, Marfan Syndrome, Mucopolysaccharidoses, Muscular Dystrophy, Sickle Cell Disease, Von Hippel-Lindau Syndrome, Congenital Deafness, Familial Hypercholesterolemia, Hemochromatosis, Neurofibromatosis type 1, Tay-Sachs Disease, Usher Syndrome, AA amyloidosis, Adrenoleukodystrophy, Ehlers-Danlos Syndrome, Lysosomal disorders, and/or Mitochondrial disorders.

In some aspects, the disease or disorder is an ocular disease. In some aspects, the ocular disease is glaucoma, age-related macular degeneration, inflammatory retinal disease, retinal vascular disease, diabetic retinopathy, uveitis, rosacea, Sjogren's syndrome, retinitis pigmentosa, retinoschisis, Stargardt disease, Leber congenital amaurosis, or neovascularization in proliferative retinopathy.

### 2. Pharmaceutical Compositions

In certain aspects, the constructs, vectors, particles, polypeptides, polynucleotides, and/or compositions (collectively described as “agents”) for use in the methods, such as methods of targeted pseudouridine installation, are suitably contained in a pharmaceutically acceptable carrier. In some aspects, the carrier is non-toxic, biocompatible and is selected so as not to detrimentally affect the biological activity of the agent. In some aspects, agents may be formulated into preparations for local delivery (i.e. to a specific location of the body, such as brain tissues, muscle tissue, fat tissue, etc.) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

In some aspects, suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

In certain aspects, the carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In some aspects, solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

In some aspects, non-limiting examples of non-aqueous solvents are propylene glycol, polyethylene glycol, vegetable oil and injectable organic esters such as ethyloleate. In some aspects, non-limiting examples of aqueous carriers include water, alcoholic/aqueous solutions, saline solutions, parenteral vehicles such as sodium chloride, Ringer's dextrose, etc. In some aspects, intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

In certain aspects, formulations comprising constructs described herein and/or co-administered formulations may be suitable for oral administration. In some aspects, oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### IV. Kits

Certain aspects of the present disclosure also concern kits containing compositions of the disclosure or compositions to implement methods disclosed herein. In some aspects, disclosed are kits that can be used to install pseudouridine in a target RNA. In some aspects, disclosed are kits that can be used to quantify and/or detect pseudouridine installation in a target RNA. In some aspects, a kit may also include additional components that are useful for purifying, amplifying, or sequencing RNA or DNA, or for other applications of the present disclosure as described herein.

The kit may optionally provide additional components that are useful in the procedure. These optional components include buffers, capture reagents, developing reagents, labels, reacting surfaces, means for detection, control samples, instructions, and interpretive information. In certain aspects, a kit contains, contains at least, or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein.

Kits may comprise components, which may be individually packaged or placed in a container, such as a tube, bottle, vial, syringe, or other suitable container means.

Individual components may also be provided in a kit in concentrated amounts; in some aspects, a component is provided individually in the same concentration as it would be in a solution with other components. Concentrations of components may be provided as 1×, 2×, 5×, 10×, or 20× or more.

In certain aspects, negative and/or positive control nucleic acids, probes, and inhibitors are included in some kit aspects. In addition, a kit may include a sample that is a negative or positive control, for example a nucleic acid that does not comprise a pseudouridine may be included as a negative control and a nucleic acid that does comprise a pseudouridine may be included as a positive control.

It is specifically contemplated that a kit of the present disclosure may exclude any one or more of the described components in certain aspects.

### EXAMPLES

The following examples are included to demonstrate certain aspects of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific aspects which are disclosed and still obtain a like or similar result without departing from the spirit and scope of the invention.

### Example 1—Ψ(Peudouridine) Sites were Enriched in Rare Codons in Mammalian mRNA

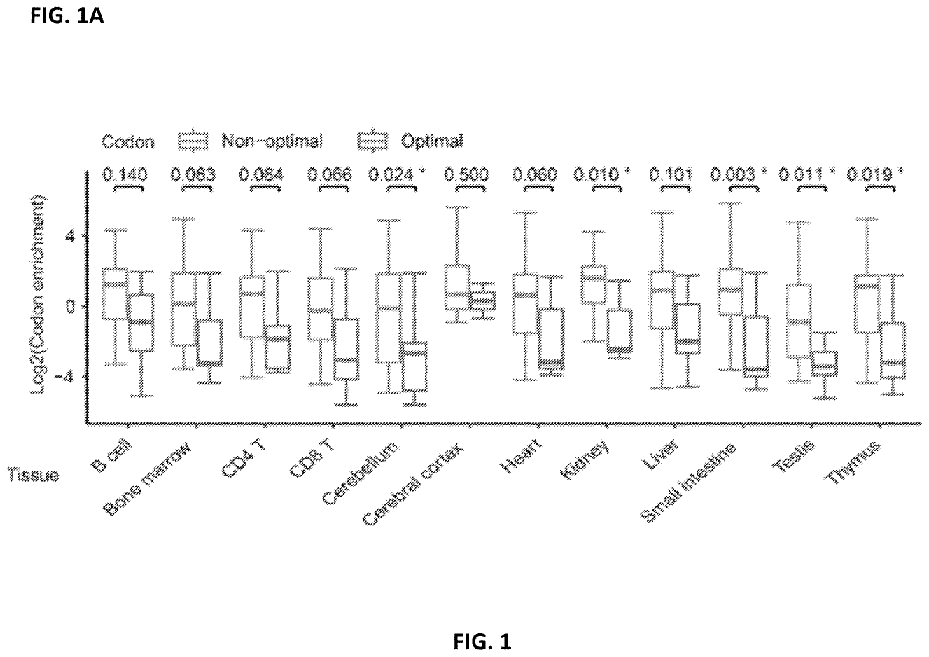

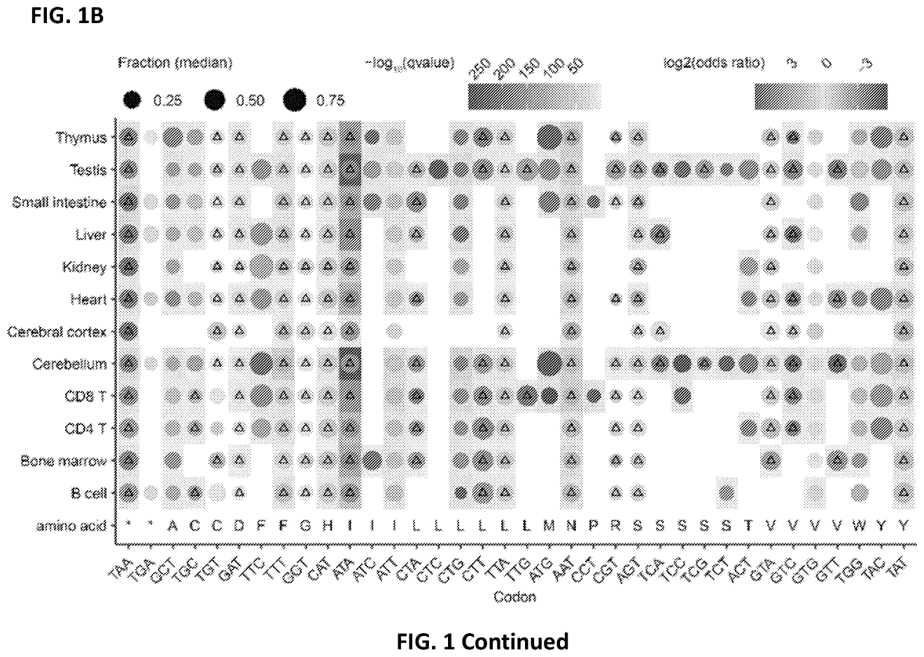

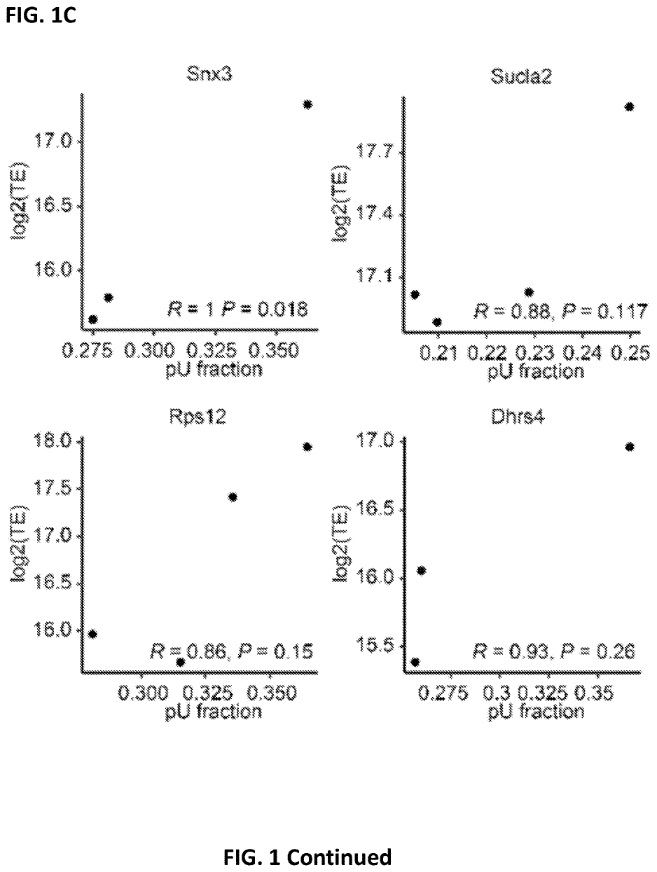

The inventors examined Ψ sites obtained from BID-seq in 12 different mouse tissues and calculated the enrichment of Ψ for each T-containing codon (i.e., U-containing codon in RNA). The inventors discovered that Ψ sites were found to be more enriched in nonoptimal codons (i.e., rare codons) despite their overall low abundance in the genome. These findings suggested a stronger propensity for Ψ modification in rare codons relative to non-rare codons (see FIG. 1A). To further examine which rare codon had a stronger propensity to be Ψ modified, the inventors compared the enrichment of all individual T-containing codons from mRNAs in 12 different mouse tissues. The results showed that numerous rare codons were enriched in Ψ in myriad tissues, and that at least six rare codons were enriched in Ψ in all 12 of the mouse tissues tested. These rare codons included TTT, CAT, ATA, TTA, AAT, and TAT. Among these rare codons ATA shows the greatest Ψ enrichment (see FIG. 1B). The inventors then plotted the Ψ fraction versus protein synthesis efficiency, which was calculated as the protein intensity detected by mass spectrometry (see e.g., PMID: 21183079, which is incorporated herein by reference in its entirety for the purposes described herein) normalized by the respective mRNA levels. Notably, Ψ in ATA codons was found to positively correlate with protein translation efficiency (see FIG. 1C). Thus, the inventors determined that as an exemplary codon, the ATA codon could be utilized to validate the discovery that site-specific engineered Ψ installation could increase protein expression.

Ribosome profiling is performed to confirm the above described observation that Ψ promotes the ribosome decoding rate at rare codons.

### Example 2—Targeted Peudouridine Installation Using Targeting Element Linked Peudouridine Synthases

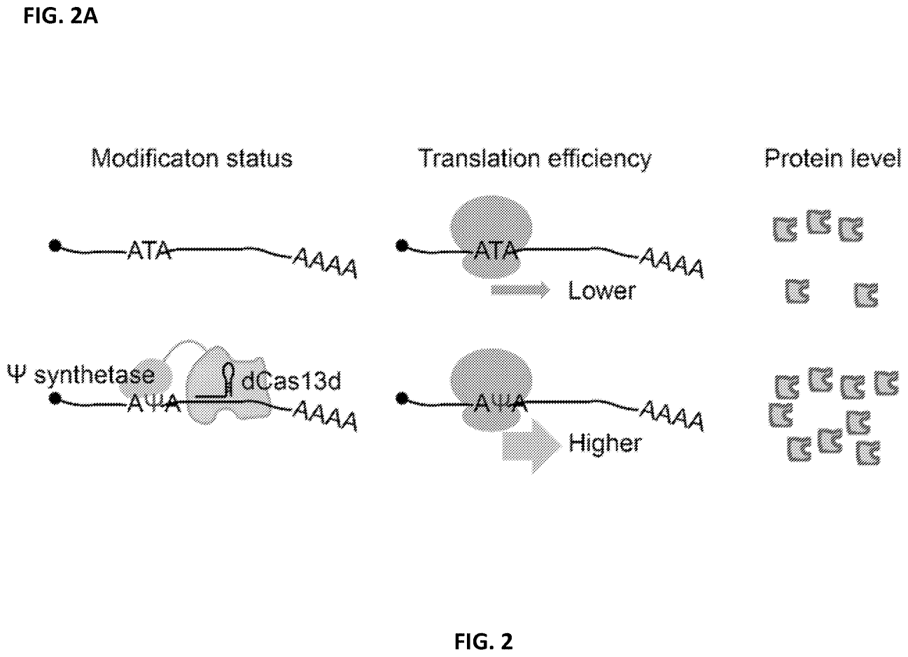

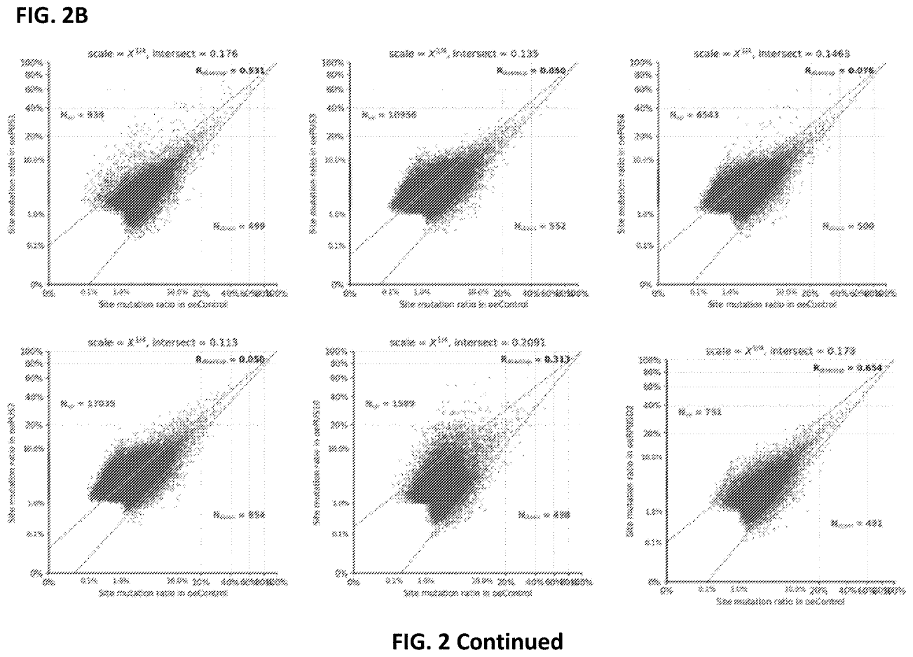

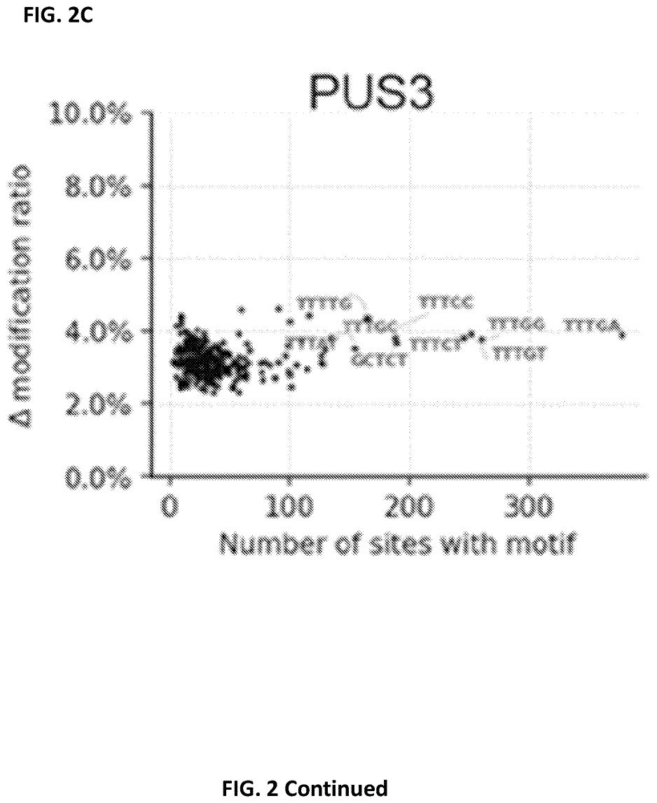

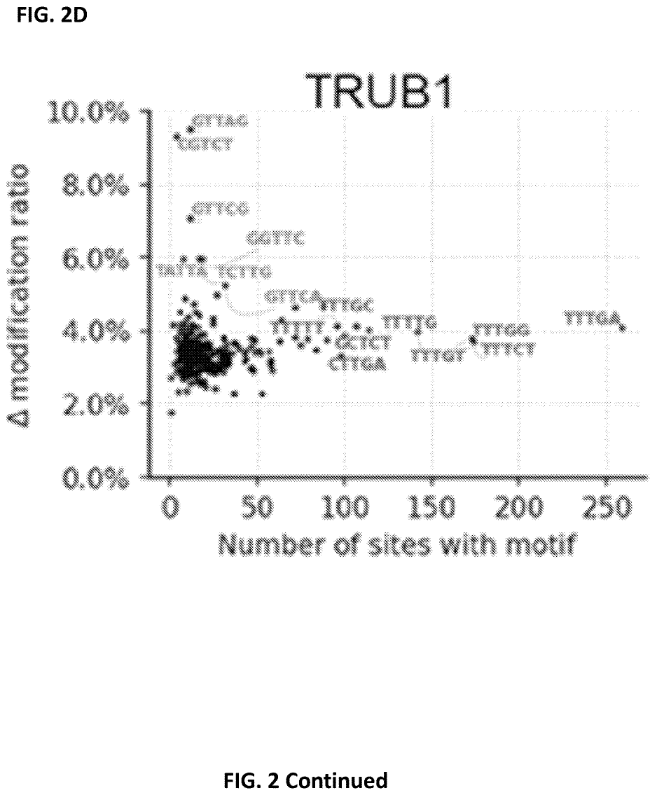

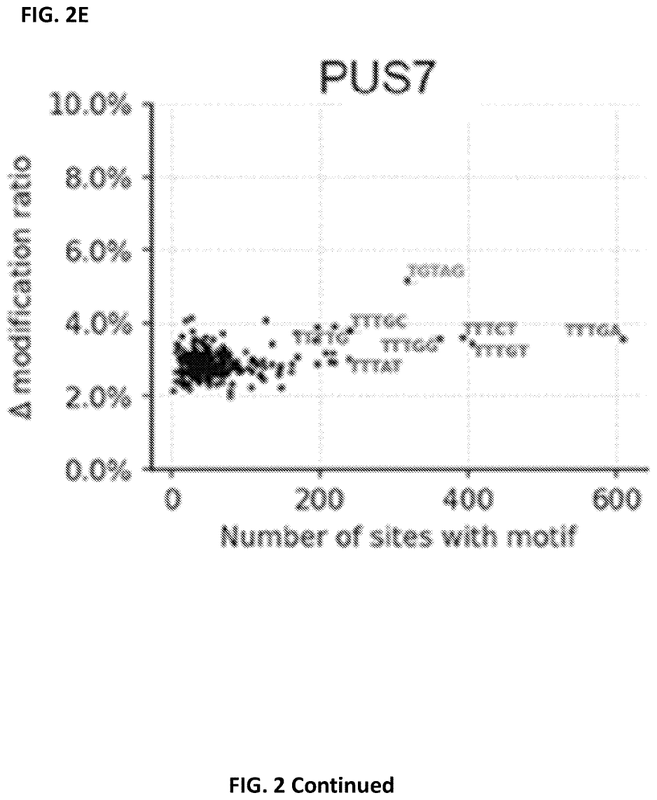

To test the causal relation between Ψ and protein expression, the inventors tethered a catalytically inactivated (“dead”) Cas13d (dCas13d) (SEQ ID NO: 20) to individual pseudouridine synthase (PUS) proteins for the programmable site-specific installation of Ψ on mRNA (see FIG. 2A). To gain a higher Ψ installation efficiency in mRNA, the inventors first identified the PUS enzymes that could catalyze Ψ in mRNAs. BID-seq was performed on human HeLa cells transiently overexpressing each of PUS1 (SEQ ID NO: 2), PUS3 (SEQ ID NO: 4), PUS4 (TRUB1) (SEQ ID NO: 6), PUS7 (SEQ ID NO: 14), PUS10 (SEQ ID NO: 16), and RPUSD2 (SEQ ID NO: 18), respectively. By comparing the Ψ fractions between the control and overexpressed (OE) sample pairs for each PUS, the inventors observed a large amount of hypermodified sites in PUS3, TRUB1, and PUS7 OE samples. The results suggested that these PUS enzymes could effectively install Ψ on mammalian mRNA (see FIG. 2B). Among these three PUS enzymes, the inventors sought to identify the enzyme with the greatest motif diversity. By identifying and selecting the PUS enzyme with the greatest motif diversity, the inventors could potentially reduce target site sequence bias. The inventors analyzed the difference in motif frequency and modification fraction of all confident mRNA Ψ sites between the control and the OE sample pairs for PUS3, TRUB1, and PUS7 respectively. TRUB1 demonstrated the highest modification activity and greatest motif diversity (see FIG. 2D), with motifs GUUAG, CGUCU, GUUCG, GGUUC, UAUUA, UCUUG, and GUUCA all showing relatively high modification levels; in contrast, PUS7 showed lower activity with a site-specific UGUAG motif bias (see FIG. 2E), while PUS3 showed lower activity with no site-specific motif bias (see FIG. 2C). Therefore, the inventors tethered (i.e., linked) dCas13d to TRUB1, creating a new protein suitable for site-directed introduction of Ψ to desired codons, such as rare codons and/or premature stop codons.

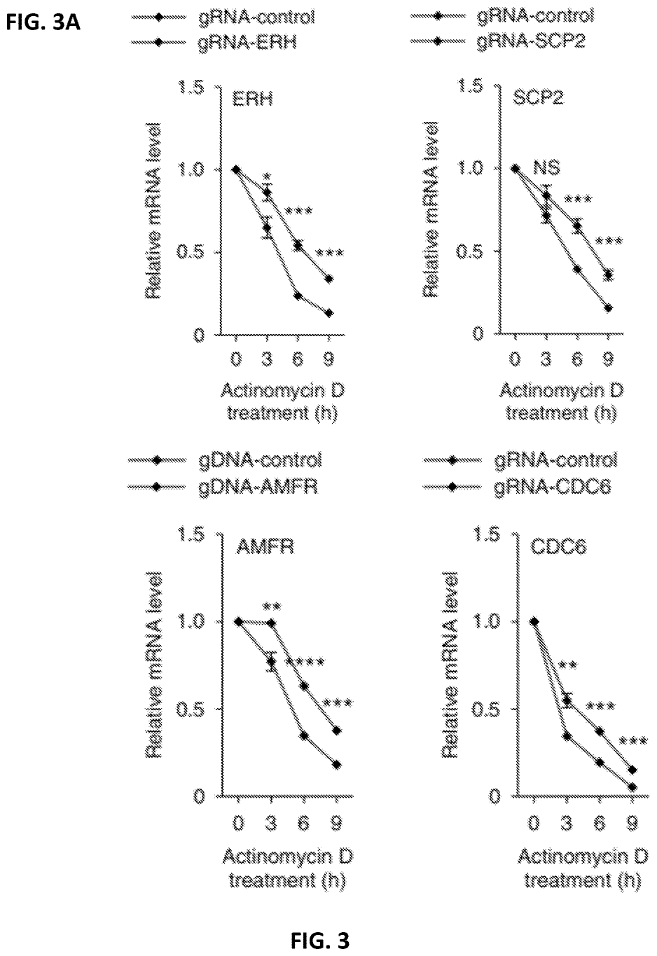

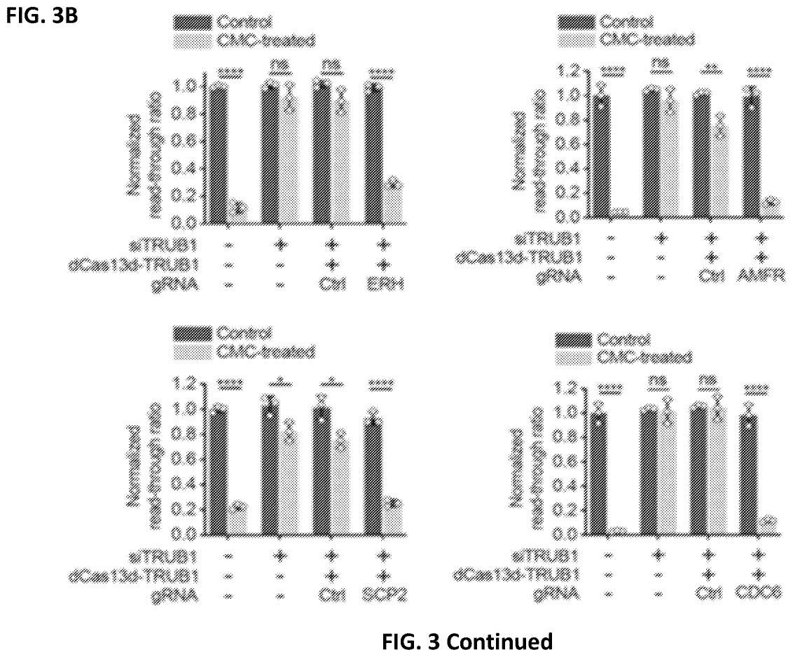

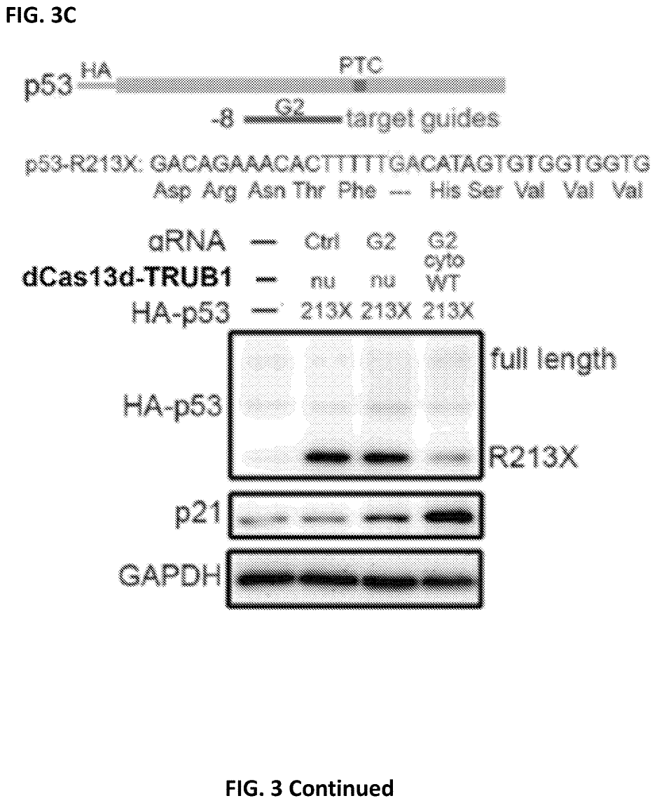

To validate the programmable Ψ deposition by the dCas13d-TRUB1 system, the inventors examined mRNA stability changes after Ψ deposition, as it has been reported that endogenous Ψ can stabilize mRNA through unknown mechanism(s) (see e.g., PMID: 25219674, PMID: 36302989, each of which are incorporated herein by reference in their entirety for the purposes described herein). The inventors designed gRNAs targeting four exemplary genes ERH, SCP2, AMFR, and CDC6, each of which were found by the inventors to comprise native Ψ sites installed by TRUB1 (e.g., as identified in the aforementioned HeLa cell BID-seq data). Endogenous-TRUB1 was knocked down in HeLa cells using RNA interference, and in these same cells transgenic dCas13d-TRUB1 overexpression was coupled with control guide RNA or ERH, SCP2, AMFR, or CDC6 specific guide RNA (SEQ ID NOs: 21-24, respectively). The data showed prolonged mRNA lifetime after Ψ restoration by guided dCas13d-TRUB1, confirming target specific Ψ deposition to these mRNAs (see FIG. 3A). To further validate the introduction of Ψ to the targeted region in the mRNA, the inventors performed N-cyclohexyl-N′-(2-morpholinoethyl) carbodiimide methyl-p-toluenesulfonate (CMC)-based RT with quantitative PCR (RT-qPCR). In general, the CMC treatment reduces the SuperScript III Reverse Transcriptase readthrough ratio at the Ψ modified sites. The inventors observed reduced readthrough ratios in the samples expressing dCas13d-TRUB1 when compared to the endogenous-TRUB1 KD samples, these results suggested base resolution precision of the engineered Ψ deposition system (see FIG. 3B).

To test if the installed Ψ could regulate translation, the inventors then targeted the dCas13d-TRUB1 system to the premature stop codon found in the p53 mutant gene, p53-R213X. In test conditions relative to controls, expression of the full-length p53 and significant reductions of the truncated p53-R213X protein were observed (see FIG. 3C). Consistently, the expression of the downstream p21 was also found to be upregulated (see FIG. 3C). Based on these results, it was concluded that Ψ installation at stop codons could facilitate transcript readthrough, and restoration of full length protein production.

### Example 3—Targeting of Rare Codons Promoted Protein Translation

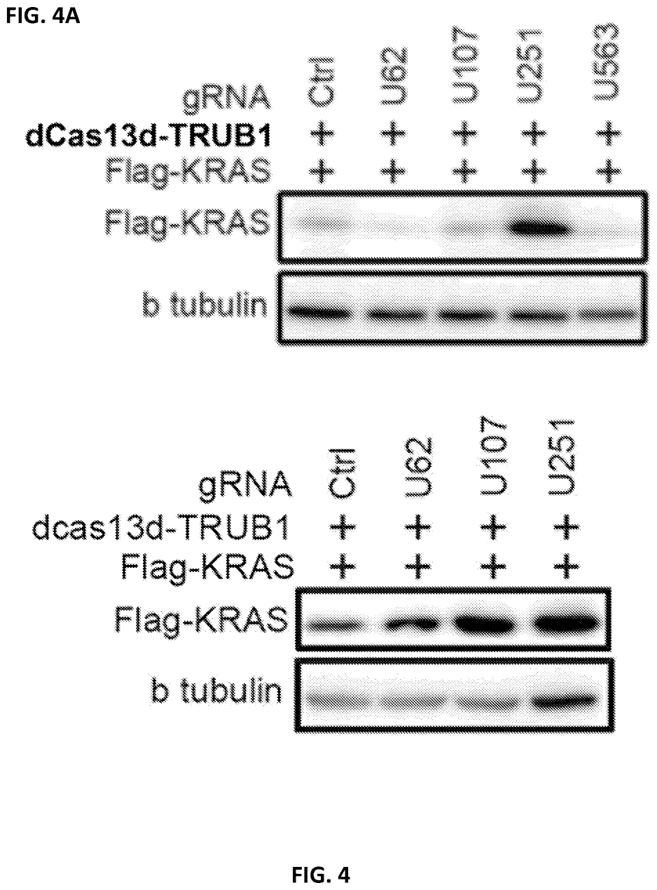

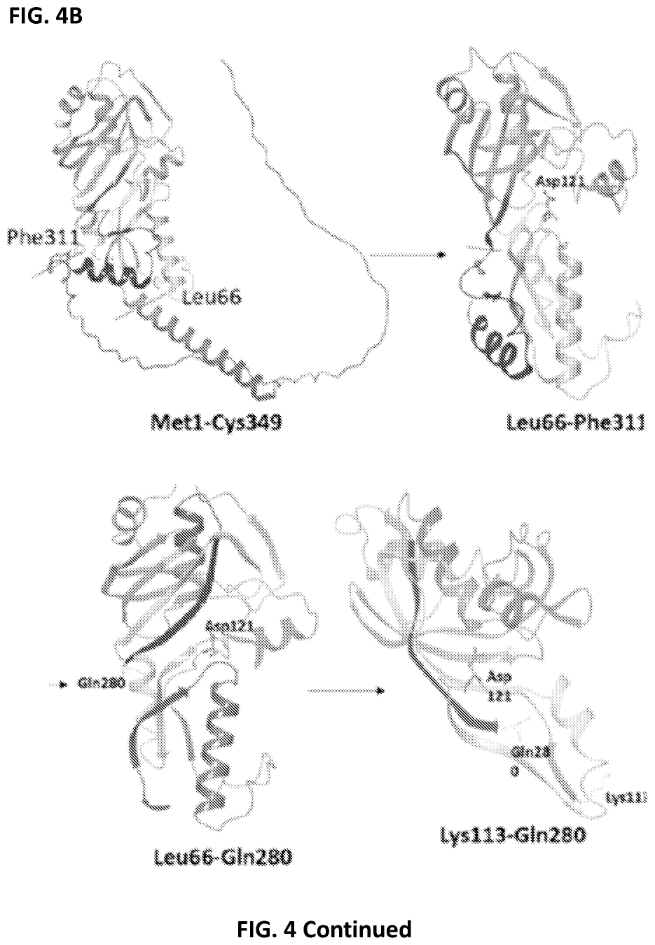

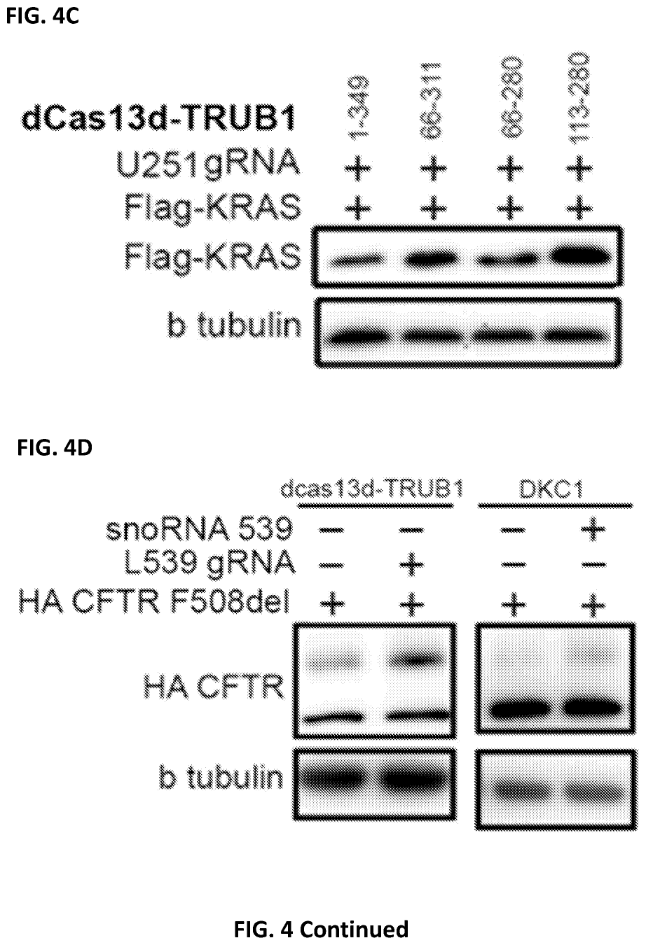

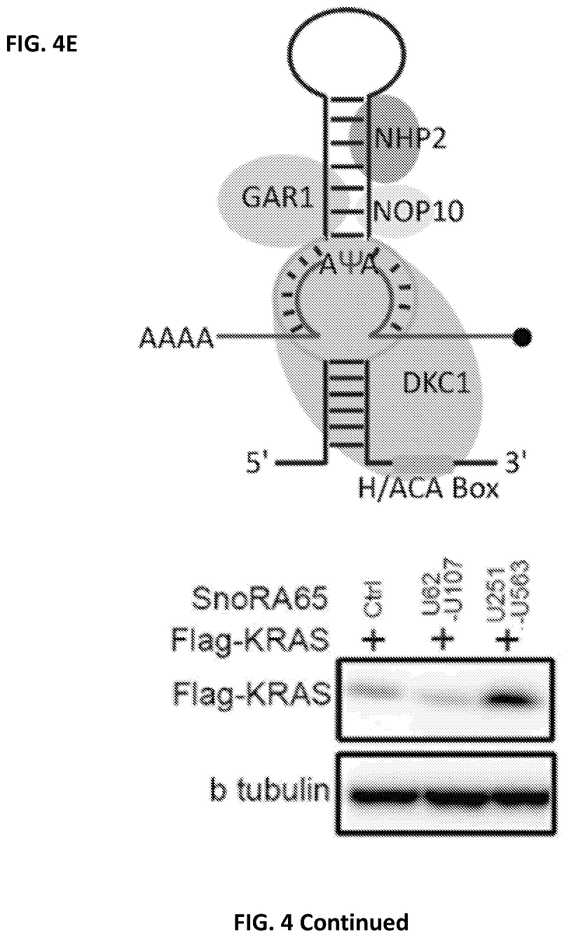

Bolstered by the above results, the Inventors then sought to determine whether targeting of rare codons, such as ATA, could increase the levels of protein translated from the mRNA. To exemplify this characteristic, the inventors used KRAS as the model gene. The inventors tested whether targeting dCas13d-TRUB1 to any one of 4 specific coding ATA sites in KRAS, U62, U107, U251, and U563 (see e.g., SEQ ID NOs: 32-34), could increase KRAS protein expression. Surprisingly, the results showed that targeting of dCas13d-TRUB1 to the KRASmRNA, particularly at U251 of the KRAS gene, dramatically increased KRAS protein levels (see FIG. 4A).

In light of the results provided in Example 2, FIG. 2D, the inventors sought to further decrease the potential for sequence bias caused by TRUB1 motif preferences. While the TRUB1 protein showed relatively low levels of motif sequence bias, the system could potentially be refined further. The inventors created various truncated TRUB1 proteins (see e.g., SEQ ID NOs: 8, 10, and 12, respectively) to determine if gene expression levels could be further increased and/or if the number of potential target RNA motif sequences accessible to the PUS comprising system could be further increased. A dCas13d protein (SEQ ID NO: 20) was linked to the different versions of truncated TRUB1 (see FIG. 4B) via an XTEN derived peptide linker (SEQ ID NO: 39). Among the different variants tested, the variant comprising only the catalytic domain of TRUB1 showed the highest levels of KRAS protein expression upregulation when compared to the full-length protein and/or other variants tested (see FIG. 4C). The inventors then tested the dCas13d::TRUB1 variants and DKC1-snoRNA based systems with alternative model genes, such as the F508del CFTR model. Again, the inventors observed increased levels of protein expression in samples treated with variant dCas13d::TRUB1(Lys113-Gln280) and DKC1-snoRNA (see FIG. 4D). Together, these results suggested the stand-alone TRUB1 catalytic domain produced a higher increase in target mRNA expression relative to full length TRUB1 protein. Furthermore, these results showed increased levels of CFTR protein when utilizing the guided dCas13d-TRUB1 Lys113-Gln280 variant system relative to the snoRNA-DKC1 system.

Furthermore, the inventors recapitulated these results using a recently published snoRNA-DKC1 system (see FIG. 4E) (PMID 36521489 and PMID 36764303, each of which are incorporated herein by reference in their entirety for the purposes described herein). The inventors also observed upregulated KRAS protein expression when cells were treated with snoRA65 targeting U251 and U563. These results suggested that protein expression upregulation was promoted by the targeting of rare codons, such as ATA, rather than a CRISPR-based bias.

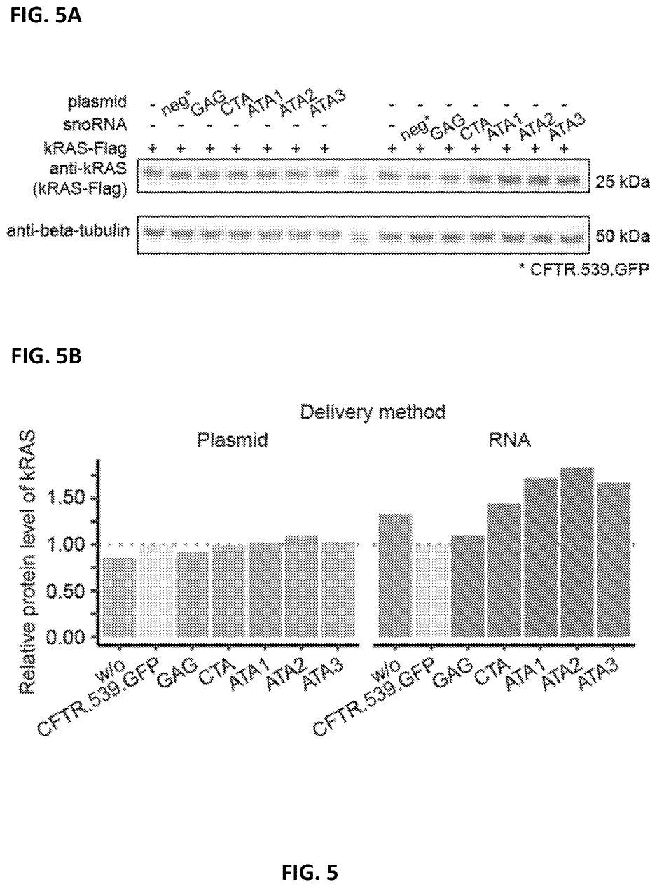

Delivery of engineered-snoRNA targeting rare codons promoted protein expression: Driven by the above results, the Inventors committed to investigate whether the endogenous DKC1 system of a cell can be leveraged using engineered snoRNAs. The Inventors engineered snoRNAs that target the GAG, CTA, ATA1, ATA2, and ATA3 codons in a KRAS-Flag reporter gene. An engineered snoRNA targeting the CFTR 1539 and GFP (neg*, CFTR.539.GFP) served as an off-target negative control. Engineered snoRNAs were delivered either as plasmids for intracellular expression or directly as RNAs (e.g., in vitro transcribed). The results showed that direct delivery of engineered-snoRNA promoted protein expression of the targeted gene (FIG. 5A-5B). However, the cellular-expressed engineered-snoRNA failed to promote the protein expression (FIG. 5A-5B). Engineered-snoRNA was in vitro transcribed using MEGAshortscript™ T7 Transcription Kit (#AM1354, Fisher Scientific). The transcribed RNA product was delivered using Lipofectamine™ RNAiMAX Transfection Reagent (#13778150, Thermo Fisher). For each transfection, 30 pmol of transcribed RNA was used per well of cells grown in a 6-well plate.

In summary, the inventors have created methods and compositions that can be utilized to modulate protein expression (e.g., upregulate or potentially downregulate) protein expression through site-specific targeting of mRNA. Without wishing to be bound by theory, targeting of pseudouridine synthases to target mRNA sites may allow for programmable Ψ installation at specific rare codons. Without wishing to be bound by theory, the technologies disclosed herein may introduce Ψ at a single-nucleotide resolution, and may regulate protein expression of a specific gene (including expression of specific transcripts) at the translational level without altering the underlying genomic sequence. Therapeutically, the technologies disclosed herein may at least provide means to rescue diseases associated with haploinsufficiency, e.g., by upregulating translation to recover protein levels to physiologically acceptable levels.

All of the methods disclosed and claimed herein can be made and executed without undue experimentation in light of the present disclosure. While the compositions and methods of this invention have been described in terms of certain aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

All references cited herein, including patent applications, patent publications, and Accession numbers, to the extent that they provide exemplary procedural or other details supplementary to those set forth herein, are specifically herein incorporated by reference in their entirety, as if each individual reference were specifically and individually indicated to be incorporated by reference.

What is claimed is:

1. A composition comprising, a pseudouridine synthase protein linked to a targeting protein.

2. The composition of claim 1, wherein the targeting protein comprises an RNA-guided protein.

3. The composition of claim 1 or 2, wherein the targeting protein comprises a clustered regularly interspaced short palindromic repeats (CRISPR) Associated (Cas) Protein

4. The composition of claim 3, wherein the Cas protein endonuclease enzymatic activity is non-functional.

5. The composition of claim 3 or 4, wherein the Cas protein comprises a dCas13d protein.

6. The composition of any one of claims 3 to 5, wherein the Cas protein comprises an amino acid sequence identity at least 80%, 85%, 90%, 95%, 99%, or 100% identical to SEQ ID NO: 20.

7. The composition of any one of claims 1 to 6, wherein the pseudouridine synthase protein comprises PUS4 (TRUB1), PUS1, PUS3, PUS7, PUS10, and/or RPUSD2.

8. The composition of any one of claims 1 to 7, wherein the pseudouridine synthase protein comprises an amino acid sequence at least 80%, 85%, 90%, 95%, 99%, or 100% identical to one or more of SEQ ID NOs: 6, 8, 10, 12, 2, 4, 14, 16, or 18.

9. The composition of any one of claims 1 to 8, wherein the pseudouridine synthase protein comprises, consists essentially of, or consists of a TRUB1 protein and/or catalytic portion thereof.

10. The composition of claim 9, wherein the TRUB1 catalytic portion comprises, consists essentially of, or consists of amino acids 1-349, 66-311, 66-280, or 113-280 of the TRUB1 protein.

11. The composition of any one of claims 1 to 10, wherein the pseudouridine synthase protein consists of or consists essentially of a protein comprising an amino acid sequence at least 80%, 85%, 90%, 95%, 99%, or 100% identical to SEQ ID NO: 6, 8, 10, or 12.

12. The composition of any one of claims 1 to 11, further comprising one or more guide RNA (gRNA) molecules that target a messenger RNA (mRNA) molecule associated with a target gene.

13. A composition comprising one or more targeting RNA molecules that target a messenger RNA (mRNA) molecule associated with a target gene.

14. The composition of claim 13, wherein the targeting RNA is a small nucleolar RNA (snoRNA) or a guide RNA (gRNA).

15. The composition of claim 14, wherein the snoRNA is in vitro transcribed RNA.

16. The composition of any one of claim 13 to 15, wherein the targeting RNA targets a gene associated with diseases and/or disorders characterized by haploinsufficiency, gene downregulation, and/or gene translation inhibiting polymorphisms.

17. The composition of any one of claims 13 to 16, wherein the gene translation inhibiting polymorphisms comprise one or more of a premature stop codon, rare codon, and/or missense codon mutation.

18. The composition of any one of claims 13 to 17, wherein the targeting RNA targets a gene comprising one or more rare codons.

19. The composition of claim 18, wherein the rare codon is ATA, TTT, CAT, TTA, AAT, or TAT.

20. The composition of claim 19, wherein the rare codon is ATA.

21. The composition of any one of claims 18 to 20, wherein the gene comprises more than one rare codon.

22. The composition of any one of claims 18 to 21, wherein the gene comprises more than one different rare codon.

23. The composition of any one of claims 13 to 22, wherein the targeting RNA molecule targets a transcript associated with any of genes KRAS, p53, DMD, HBB, IDUA NF1, CDC6, AMFR, SCP2, ERH, CFTR, GLMN, POT1, TBK1, KRIT1, NBN, FANCB, DSC2, LIG4, ATP2C1, PGAP1, RB1, MSH2, RASA1, DSG1, KIF11, CHL1, RAD50, ATP7A, PHIP, SI, APC, ATM, and/or BRCA2.

24. The composition of any one of claims 13 to 23, wherein the targeting RNA molecule comprises a polynucleotide sequence at least 85%, 90%, 95%, or 100% identical to any one or more of SEQ ID NOs: 21-28.

25. The composition of any one of claims 13 to 24, wherein the target mRNA is a transcript associated with the p53 gene.

26. The composition of claim 25, wherein the target mRNA associated with the p53 gene comprises a non-sense mutation.

27. The composition of claim 26, wherein the non-sense mutation comprises R213X.

28. The composition of any one of claims 13 to 24, wherein the target mRNA is a transcript associated with the KRAS gene.

29. The composition of any one of claims 13 to 24, wherein the targeting RNA targets uracil (U) 62, U107, U251, U563, CTA, ATA1, ATA2, ATA3, or a combination thereof of an mRNA associated with the KRAS gene.

30. A composition comprising a pseudouridine synthase protein linked to a targeting protein according to any one of claims 1 to 24, and a guide RNA according to any one of claims 13 to 24.

31. A method of modulating translation of a target mRNA comprising, contacting one or more site-specific rare codons in the target mRNA with a) one or more site-specific targeting elements, and/or b) one or more of a pseudouridine synthase protein and/or one or more box H/ACA small nucleolar ribonucleoprotein (H/ACA snoRNP) complex components.

32. The method of claim 31, wherein b) comprises a pseudouridine synthase protein fused to a targeting protein.

33. The method of claim 31, wherein b) comprises a DKC1 complex.

34. The method of claim 31, wherein b) comprises DKC1 isoform 3.

35. The method of claim 31 or 32, wherein a) comprises a targeting protein and/or polypeptide.

36. The method of any one of claims 31 to 35, wherein a) comprises an RNA-guided protein.

37. The method of any one of claims 31 to 36, wherein the targeting element comprises a clustered regularly interspaced short palindromic repeats (CRISPR) Associated (Cas) Protein.

38. The method of claim 37, wherein the Cas protein endonuclease enzymatic activity is non-functional.

39. The method of claim 38 or 39, wherein the Cas protein comprises a dCas13d protein.

40. The method of any one of claims 37 to 39, wherein the Cas protein comprises a sequence identity at least 80%, 85%, 90%, 95%, 99%, or 100% identical to SEQ ID NO: 20.

41. The method of any one of claims 31 to 40, wherein the pseudouridine synthase protein comprises PUS4 (TRUB1), PUS1, PUS3, PUS7, PUS10, and/or RPUSD2.

42. The method of any one of claims 31 to 41, wherein the pseudouridine synthase protein comprises a sequence at least 80%, 85%, 90%, 95%, 99%, or 100% identical to one or more of SEQ ID NOs: 6, 8, 10, 12, 2, 4, 14, 16, or 18.

43. The method of any one of claims 31 to 42, wherein the pseudouridine synthase protein comprises, consists essentially of, or consists of a TRUB1 protein and/or catalytic portion thereof.

44. The composition of claim 43, wherein the TRUB1 catalytic portion comprises, consists essentially of, or consists of amino acids 1-349, 66-311, 66-280, or 113-280 of the TRUB1 protein.

45. The method of any one of claims 31 to 44, wherein the pseudouridine synthase protein consists of or consists essentially of a protein comprising a sequence at least 80%, 85%, 90%, 95%, 99%, or 100% identical to SEQ ID NOs: 6, 8, 10, or 12.

46. The method of any one of claims 31 to 45, wherein the pseudouridine synthase protein consists of or consists essentially of a protein comprising a sequence at least 80%, 85%, 90%, 95%, 99%, or 100% identical to SEQ ID NO: 12.

47. The method of any one of claims 31 to 46, wherein the targeting element comprises one or more guide RNA (gRNA) molecules.

48. The method of claim 47, wherein the gRNA targets a gene associated with diseases and/or disorders characterized by haploinsufficiency, gene downregulation, and/or gene translation inhibiting polymorphisms.

49. The method of claim 48, wherein the gRNA targets a gene comprising one or more rare codons.

50. The method of claim 49, wherein the gene comprises more than one different rare codon.

51. The method of any one of claims 31 to 50, wherein the rare codon is ATA, TTT, CAT, TTA, AAT, or TAT.

52. The method of any one of claims 31 to 51, wherein the rare codon is ATA.

53. The method of any one of claims 31 to 52, wherein the target mRNA comprises more than one rare codons.

54. The method of any one of claims 31 to 53, wherein the target mRNA is a transcript associated with any of genes KRAS, p53, DMD, HBB, IDUA NF1, CDC6, AMFR, SCP2, ERH, CFTR, GLMN, POT1, TBK1, KRIT1, NBN, FANCB, DSC2, LIG4, ATP2C1, PGAP1, RB1, MSH2, RASA1, DSG1, KIF11, CHL1, RAD50, ATP7A, PHIP, SI, APC, ATM, and/or BRCA2.

55. The method of any one of claims 31 to 54, wherein the targeting element comprises a polynucleotide sequence at least 85%, 90%, 95%, or 100% identical to any one or more of SEQ ID NOs: 21-28.

56. The composition of any one of claims 31 to 54, wherein the target mRNA is a transcript associated with the p53 gene.

57. The composition of claim 56, wherein the target mRNA associated with the p53 gene comprises a non-sense mutation.

58. The composition of claim 57, wherein the non-sense mutation comprises R213X.

59. The method of any one of claims 31 to 55, wherein the target mRNA is a transcript associated with the KRAS gene.

60. The composition of any one of claims 31 to 55, wherein the targeting RNA targets uracil (U) 62, U107, U251, U563, CTA, ATA1, ATA2, ATA3, or a combination thereof of an mRNA associated with the KRAS gene.

61. The method of claim 59 or 60, wherein the targeting element comprises a polynucleotide sequence at least 85%, 90%, 95%, or 100% identical to any one or more of SEQ ID NOs: 25-28.

62. The method of claim 61, wherein the targeting element comprises a polynucleotide sequence at least 85%, 90%, 95%, or 100% identical to any one or more of SEQ ID NOs: 27.

63. The method of any one of claims 59 to 62, wherein targeting the KRAS mRNA increases KRAS protein levels.

64. The method of claim 31, wherein the targeting element comprises an engineered small nucleolar RNA (snoRNA).

65. The method of claim 64, wherein the engineered snoRNA is in vitro transcribed.

66. The method of claim 64 or 65, wherein the engineered snoRNA targets a transcript associated with any of genes KRAS, p53, DMD, HBB, IDUA NF1, CDC6, AMFR, SCP2, ERH, CFTR, GLMN, POT1, TBK1, KRIT1, NBN, FANCB, DSC2, LIG4, ATP2C1, PGAP1, RB1, MSH2, RASA1, DSG1, KIF11, CHL1, RAD50, ATP7A, PHIP, SI, APC, ATM, and/or BRCA2.

67. The method of claim 64 or 66, wherein the engineered snoRNA targets a transcript associated with KRAS.

68. The method of claim 67, wherein the engineered sno-RNA comprises, consists essentially of, or consists of a polynucleotide sequence at least 85%, 90%, 95%, or 100% identical to any one or more of SEQ ID NOs: 37-45.

69. The method of claim 67 or 68, wherein the engineered snoRNA targets uracil (U) 62, U107, U251, U563, CTA, ATA1, ATA2, ATA3, or a combination thereof of an mRNA associated with the KRAS gene.

70. A method of treating a disease and/or disorder in an individual comprising, providing the individual in need thereof with a composition according to any one of claims 1-30.

71. A method of treating a disease and/or disorder in an individual comprising, performing the method of modulating translation levels of mRNA according to any one of claims 31 to 68 on the individual in need thereof.

72. The method of claim 70 or 71, wherein the disease and/or disorder is cancer.

73. The method of any one of claims 70 to 72, wherein the disease and/or disorder is characterized by haploinsufficiency.

74. A kit comprising a composition of any one of claims 1 to 30.

75. Use of the composition, method, or kit according to any one of claims 1 to 74, as a medicament, means for treatment and/or prevention of a disease, means for diagnosis, and/or medical research tool.


SEQUENCE LISTING (45 sequences)

SEQ ID NO: 1 - RNA, 1281 nt, Homo sapiens
  source 1..1281: mol_type = mRNA
atgggcctccagcttcgcgcgctgttgggagccttcggacggtggaccctgcgcctgggaccgcgtccgtcctgctcgccgcgcatggccgggaacgcggagccgccgcccgccggagccgcatgcccccaggaccggaggtcctgcagcggccgggccgggggcgaccgcgtctgggaggacggagaacatccggcgaagaagctcaagagcggtggcgacgaggagcggcgcgagaagccgcccaagcggaagatcgtgctgctcatggcctattcgggcaagggctaccacggcatgcagaggaatgtcgggtcctcacaattcaaaacaattgaagatgacttggtgtccgccctcgtccggtcaggctgtattcctgaaaatcatggtgaggacatgaggaaaatgtccttccagcgctgcgcccggacagacaagggtgtgtccgcagccggccaggtggtatccctgaaggtgtggctgattgacgacattctagaaaagatcaacagccaccttccctctcacattcggattctgggactgaagcgggtcacgggcgggtttaactccaagaacagatgtgatgccaggacctattgctacctgctgcccacgtttgcctttgcgcacaaggaccgggacgttcaggatgagacctaccgcctgagcgccgagacgctgcagcaggtcaacaggctcctggcctgctacaagggcacgcacaacttccacaatttcacctcgcagaaggggccgcaggatcccagtgcctgccgctacatcctggagatgtactgcgaggaaccctttgtgcgggagggcctggagtttgcggtgatcagggtgaagggccagagcttcatgatgcatcagatccggaagatggtcggcctggtggtggccattgtgaagggttatgcccctgagagcgtgctggagcgcagctggggcacagagaaggtggacgtgcccaaggcgcccggactcggcctggtcctggagagggtgcacttcgagaagtacaaccagcgctttggcaacgatgggctgcatgagccgctggactgggcgcaggaggaaggaaaggtcgcagccttcaaggaggagcacatctaccccaccatcatcggcaccgagcgggacgaacgctccatggcccagtggctgagcaccttgcccatccacaacttcagtgccaccgctctcacggcaggtggcacgggcgccaaggtgcccagtcccctggaaggcagtgaaggggacggagacactgac

SEQ ID NO: 2 - protein, 427 aa, Homo sapiens
MGLQLRALLGAFGRWTLRLGPRPSCSPRMAGNAEPPPAGAACPQDRRSCSGRAGGDRVWEDGEHPAKKLKSGGDEERREKPPKRKIVLLMAYSGKGYHGMQRNVGSSQFKTIEDDLVSALVRSGCIPENHGEDMRKMSFQRCARTDKGVSAAGQVVSLKVWLIDDILEKINSHLPSHIRILGLKRVTGGFNSKNRCDARTYCYLLPTFAFAHKDRDVQDETYRLSAETLQQVNRLLACYKGTHNFHNFTSQKGPQDPSACRYILEMYCEEPFVREGLEFAVIRVKGQSFMMHQIRKMVGLVVAIVKGYAPESVLERSWGTEKVDVPKAPGLGLVLERVHFEKYNQRFGNDGLHEPLDWAQEEGKVAAFKEEHIYPTIIGTERDERSMAQWLSTLPIHNFSATALTAGGTGAKVPSPLEGSEGDGDTD

SEQ ID NO: 3 - RNA, 1443 nt, Homo sapiens
  source 1..1443: mol_type = mRNA
atggctgataatgacacagacagaaaccagactgagaagctcctaaaaagagtacgagaactggagcaagaggtgcaaagacttaaaaaggaacaggccaaaaataaggaggactcaaacattagagaaaattcatcaggagctggaaaaactaagcgtgcatttgatttcagtgctcatggccgaagacacgtagccctaagaatagcctatatgggctggggataccagggctttgctagtcaggaaaacacaaataataccattgaagagaaactgtttgaagctctaaccaagactcgactagtagaaagcagacagacatccaactatcaccgatgtgggagaacagataaaggagttagtgcctttggacaggtgatctcacttgaccttcgctctcagtttccaaggggcagggattccgaggactttaatgtaaaagaggaggctaatgctgctgctgaagagatccgttatacccacattctcaatcgggtactccctccagacatccgtatattggcctgggcccctgtagaaccaagcttcagtgctaggttcagctgccttgagcggacttaccgctattttttccctcgtgctgatttagatattgtaaccatggattatgcagctcagaagtatgttggcacccatgatttcaggaacttgtgtaaaatggatgtagccaacggtgtgattaattttcagaggactattctatctgctcaagtacagctagtgggccagagcccaggtgaggggagatggcaagaacctttccagttatgtcagtttgaagtgactggccaggcattcctttatcatcaagtccgatgtatgatggctatcctctttctgattggccaaggaatggagaagccagagattattgatgagctgctgaatatagagaaaaatccccaaaagcctcaatatagtatggctgtagaatttcctctagtcttatatgactgtaagtttgaaaatgtcaagtggatctatgaccaggaggctcaggagttcaatattacccacctacaacaactgtgggctaatcatgctgtcaaaactcacatgttgtatagtatgctacaaggactggacactgttccagtaccctgtggaataggaccaaagatggatggaatgacagaatggggaaatgttaagccctctgtcataaagcagaccagtgcctttgtagaaggagtgaagatgcgcacatataagcccctcatggaccgtcctaaatgccaaggactggaatcccggatccagcattttgtacgtaggggacgaattgagcacccacatttattccatgaggaagaaacaaaagccaaaagggactgtaatgacacactagaggaagacaatactaatttggagacaccaacgaagagggtctgtgttgacacagaaattaaaagcatcatt

SEQ ID NO: 4 - protein, 481 aa, Homo sapiens
MADNDTDRNQTEKLLKRVRELEQEVQRLKKEQAKNKEDSNIRENSSGAGKTKRAFDFSAHGRRHVALRIAYMGWGYQGFASQENTNNTIEEKLFEALTKTRLVESRQTSNYHRCGRTDKGVSAFGQVISLDLRSQFPRGRDSEDFNVKEEANAAAEEIRYTHILNRVLPPDIRILAWAPVEPSFSARFSCLERTYRYFFPRADLDIVTMDYAAQKYVGTHDFRNLCKMDVANGVINFQRTILSAQVQLVGQSPGEGRWQEPFQLCQFEVTGQAFLYHQVRCMMAILFLIGQGMEKPEIIDELLNIEKNPQKPQYSMAVEFPLVLYDCKFENVKWIYDQEAQEFNITHLQQLWANHAVKTHMLYSMLQGLDTVPVPCGIGPKMDGMTEWGNVKPSVIKQTSAFVEGVKMRTYKPLMDRPKCQGLESRIQHFVRRGRIEHPHLFHEEETKAKRDCNDTLEEDNTNLETPTKRVCVDTEIKSII

SEQ ID NO: 5 - RNA, 1047 nt, Homo sapiens
  source 1..1047: mol_type = mRNA
atggccgcttctgaggcggcggtggtgtcttcgccgtctttgaaaacagacacatcccctgtccttgaaactgcaggaacggtcgcagcaatggctgcgaccccgtcagcaagggctgcagccgcggtggttgcggccgcggccaggaccggatccgaagccagggtctccaaggccgctttggctaccaagctgctgtccttgagcggcgtgttcgccgtgcacaagcccaaagggcccacttcagccgagctgctgaatcggttgaaggagaagctgctggcagaagctggaatgccttctccagaatggaccaagaggaaaaagcagactttgaaaattgggcatggagggactctagacagcgcagcccgaggagttctggttgttggaattggaagcggaacaaaaatgttgaccagtatgttgtcagggtccaagagatatactgccattggagaactggggaaagctactgatacactagattctacggggagggtaacagaagaaaaaccttacgataaaataacacaagaagatattgaaggcattctacagaaatttactggaaatataatgcaagtgccccccctctattctgcattaaagaaagatggacaaagactttcgactttgatgaagagaggtgaagtcgtagaagcaaaacctgccaggccagtgactgtatacagtatctcccttcaaaaattccagccaccatttttcacattagatgttgaatgtggaggaggtttttatatcagaagcttggtcagtgacattggaaaagaactatcttcctgtgccaatgtgctagagctgacccgaaccaaacagggaccatttacgctagaagaacatgcccttcctgaagacaaatggacaattgatgacattgcacagtctcttgagcattgctcatctcttttcccagcagagttggcacttaaaaaatcaaaacctgagtctaatgaacaggttttgagctgtgaatatataactctaaatgagccaaagagagaagatgatgtaattaagacgtgt

SEQ ID NO: 6 - protein, 349 aa, Homo sapiens
MAASEAAVVSSPSLKTDTSPVLETAGTVAAMAATPSARAAAAVVAAAARTGSEARVSKAALATKLLSLSGVFAVHKPKGPTSAELLNRLKEKLLAEAGMPSPEWTKRKKQTLKIGHGGTLDSAARGVLVVGIGSGTKMLTSMLSGSKRYTAIGELGKATDTLDSTGRVTEEKPYDKITQEDIEGILQKFTGNIMQVPPLYSALKKDGQRLSTLMKRGEVVEAKPARPVTVYSISLQKFQPPFFTLDVECGGGFYIRSLVSDIGKELSSCANVLELTRTKQGPFTLEEHALPEDKWTIDDIAQSLEHCSSLFPAELALKKSKPESNEQVLSCEYITLNEPKREDDVIKTC

SEQ ID NO: 7 - RNA, 741 nt, synthetic construct
  source 1..741: mol_type = other RNA
atgctgtccttgagcggcgtgttcgccgtgcacaagcccaaagggcccacttcagccgagctgctgaatcggttgaaggagaagctgctggcagaagctggaatgccttctccagaatggaccaagaggaaaaagcagactttgaaaattgggcatggagggactctagacagcgcagcccgaggagttctggttgttggaattggaagcggaacaaaaatgttgaccagtatgttgtcagggtccaagagatatactgccattggagaactggggaaagctactgatacactagattctacggggagggtaacagaagaaaaaccttacgataaaataacacaagaagatattgaaggcattctacagaaatttactggaaatataatgcaagtgccccccctctattctgcattaaagaaagatggacaaagactttcgactttgatgaagagaggtgaagtcgtagaagcaaaacctgccaggccagtgactgtatacagtatctcccttcaaaaattccagccaccatttttcacattagatgttgaatgtggaggaggtttttatatcagaagcttggtcagtgacattggaaaagaactatcttcctgtgccaatgtgctagagctgacccgaaccaaacagggaccatttacgctagaagaacatgcccttcctgaagacaaatggacaattgatgacattgcacagtctcttgagcattgctcatctcttttc

SEQ ID NO: 8 - protein, 247 aa, synthetic construct
MLSLSGVFAVHKPKGPTSAELLNRLKEKLLAEAGMPSPEWTKRKKQTLKIGHGGTLDSAARGVLVVGIGSGTKMLTSMLSGSKRYTAIGELGKATDTLDSTGRVTEEKPYDKITQEDIEGILQKFTGNIMQVPPLYSALKKDGQRLSTLMKRGEVVEAKPARPVTVYSISLQKFQPPFFTLDVECGGGFYIRSLVSDIGKELSSCANVLELTRTKQGPFTLEEHALPEDKWTIDDIAQSLEHCSSLF

SEQ ID NO: 9 - RNA, 648 nt, synthetic construct
  source 1..648: mol_type = other RNA
atgctgtccttgagcggcgtgttcgccgtgcacaagcccaaagggcccacttcagccgagctgctgaatcggttgaaggagaagctgctggcagaagctggaatgccttctccagaatggaccaagaggaaaaagcagactttgaaaattgggcatggagggactctagacagcgcagcccgaggagttctggttgttggaattggaagcggaacaaaaatgttgaccagtatgttgtcagggtccaagagatatactgccattggagaactggggaaagctactgatacactagattctacggggagggtaacagaagaaaaaccttacgataaaataacacaagaagatattgaaggcattctacagaaatttactggaaatataatgcaagtgccccccctctattctgcattaaagaaagatggacaaagactttcgactttgatgaagagaggtgaagtcgtagaagcaaaacctgccaggccagtgactgtatacagtatctcccttcaaaaattccagccaccatttttcacattagatgttgaatgtggaggaggtttttatatcagaagcttggtcagtgacattggaaaagaactatcttcctgtgccaatgtgctagagctgacccgaaccaaacag

SEQ ID NO: 10 - protein, 216 aa, synthetic construct
MLSLSGVFAVHKPKGPTSAELLNRLKEKLLAEAGMPSPEWTKRKKQTLKIGHGGTLDSAARGVLVVGIGSGTKMLTSMLSGSKRYTAIGELGKATDTLDSTGRVTEEKPYDKITQEDIEGILQKFTGNIMQVPPLYSALKKDGQRLSTLMKRGEVVEAKPARPVTVYSISLQKFQPPFFTLDVECGGGFYIRSLVSDIGKELSSCANVLELTRTKQ

SEQ ID NO: 11 - RNA, 507 nt, synthetic construct
  source 1..507: mol_type = other RNA
atgaaaattgggcatggagggactctagacagcgcagcccgaggagttctggttgttggaattggaagcggaacaaaaatgttgaccagtatgttgtcagggtccaagagatatactgccattggagaactggggaaagctactgatacactagattctacggggagggtaacagaagaaaaaccttacgataaaataacacaagaagatattgaaggcattctacagaaatttactggaaatataatgcaagtgccccccctctattctgcattaaagaaagatggacaaagactttcgactttgatgaagagaggtgaagtcgtagaagcaaaacctgccaggccagtgactgtatacagtatctcccttcaaaaattccagccaccatttttcacattagatgttgaatgtggaggaggtttttatatcagaagcttggtcagtgacattggaaaagaactatcttcctgtgccaatgtgctagagctgacccgaaccaaacag

SEQ ID NO: 12 - protein, 169 aa, synthetic construct
MKIGHGGTLDSAARGVLVVGIGSGTKMLTSMLSGSKRYTAIGELGKATDTLDSTGRVTEEKPYDKITQEDIEGILQKFTGNIMQVPPLYSALKKDGQRLSTLMKRGEVVEAKPARPVTVYSISLQKFQPPFFTLDVECGGGFYIRSLVSDIGKELSSCANVLELTRTKQ

SEQ ID NO: 13 - RNA, 1983 nt, Homo sapiens
  source 1..1983: mol_type = mRNA
atggagatgacagaaatgactggtgtgtcgctgaaacgtggggcactggttgtcgaagataatgacagtggagtcccagttgaagagacaaaaaaacagaagctgtcggaatgcagcctaaccaaaggtcaagatgggctacagaatgactttctgtccatcagtgaagacgtgcctcggcctcctgacactgtcagtactgggaaaggtggaaagaattctgaggctcagttggaagatgaggaagaagaggaggaagatggactttcagaggagtgcgaggaggaggaatcagagagttttgcagacatgatgaagcatggactcactgaggctgacgtaggcatcaccaagtttgtgagttctcatcaagggttctcgggaatcttaaaagaaagatactccgacttcgttgttcatgaaataggaaaagatggacggatcagccatttgaatgacttgtccattccagtggatgaggaggacccttcagaagacatatttacagttttgacagctgaagaaaagcagcgattggaagagctccagctgttcaaaaataaggaaaccagtgttgccattgaggttatcgaggacaccaaagagaaaagaaccatcatccatcaggctatcaaatctctgtttccaggattagagacaaaaacagaggatagggaggggaagaaatacattgtagcctaccacgcagctgggaaaaaggctttggcaaatccaagaaaacattcttggccaaaatctaggggaagttactgccacttcgtactatataaggaaaacaaagacaccatggatgctattaatgtactctccaaatacttaagagtcaagccaaatatattctcctacatgggaaccaaagataaaagggctataacagttcaagaaattgctgttctcaaaataactgcacaaagacttgcccacctgaataagtgcttgatgaactttaagctagggaatttcagctatcaaaaaaacccactgaaattgggagagcttcaaggaaaccacttcactgttgttctcagaaatataacaggaactgatgaccaagtacagcaagctatgaactctctcaaggagattggatttattaactactatggaatgcaaagatttggaaccacagctgtccctacgtatcaggttggaagagctatactacaaaattcctggacagaagtcatggatttaatattgaaaccccgctctggagctgaaaagggctacttggttaaatgcagagaagaatgggcaaagaccaaagacccaactgctgccctcagaaaactacctgtcaaaaggtgtgtggaagggcagctgcttcgaggactttcaaaatatggaatgaagaatatagtctctgcatttggcataatacccagaaataatcgcttaatgtatattcatagctaccaaagctatgtgtggaataacatggtaagcaagaggatagaagactatggactaaaacctgttccaggggacctcgttctcaaaggagccacagccacctatattgaggaagatgatgttaataattactctatccatgatgtggtaatgcccttgcctggtttcgatgttatctacccaaagcataaaattcaagaagcctacagggaaatgctcacagctgacaatcttgatattgacaacatgagacacaaaattcgagattattccttgtcaggggcctaccgaaagatcattattcgtcctcagaatgttagctgggaagtcgttgcatatgatgatcccaaaattccacttttcaacacagatgtggacaacctagaagggaagacaccaccagtttttgcttctgaaggcaaatacagggctctgaaaatggatttttctctacccccttctacttacgccaccatggccattcgagaagtgctaaaaatggataccagtatcaagaaccagacgcagctgaatacaacctggcttcgc

SEQ ID NO: 14 - protein, 661 aa, Homo sapiens
MEMTEMTGVSLKRGALVVEDNDSGVPVEETKKQKLSECSLTKGQDGLQNDFLSISEDVPRPPDTVSTGKGGKNSEAQLEDEEEEEEDGLSEECEEEESESFADMMKHGLTEADVGITKFVSSHQGFSGILKERYSDFVVHEIGKDGRISHLNDLSIPVDEEDPSEDIFTVLTAEEKQRLEELQLFKNKETSVAIEVIEDTKEKRTIIHQAIKSLFPGLETKTEDREGKKYIVAYHAAGKKALANPRKHSWPKSRGSYCHFVLYKENKDTMDAINVLSKYLRVKPNIFSYMGTKDKRAITVQEIAVLKITAQRLAHLNKCLMNFKLGNFSYQKNPLKLGELQGNHFTVVLRNITGTDDQVQQAMNSLKEIGFINYYGMQRFGTTAVPTYQVGRAILQNSWTEVMDLILKPRSGAEKGYLVKCREEWAKTKDPTAALRKLPVKRCVEGQLLRGLSKYGMKNIVSAFGIIPRNNRLMYIHSYQSYVWNNMVSKRIEDYGLKPVPGDLVLKGATATYIEEDDVNNYSIHDVVMPLPGFDVIYPKHKIQEAYREMLTADNLDIDNMRHKIRDYSLSGAYRKIIIRPQNVSWEVVAYDDPKIPLFNTDVDNLEGKTPPVFASEGKYRALKMDFSLPPSTYATMAIREVLKMDTSIKNQTQLNTTWLR

SEQ ID NO: 15 - RNA, 1587 nt, Homo sapiens
  source 1..1587: mol_type = mRNA
atgttcccactgactgaggaaaacaagcatgtggcccagttgttgctcaatactggtacttgtccaagatgtatcttcagattctgtggtgtggattttcatgcaccttacaaacttccatacaaggagttgctcaatgaactacagaaatttctggaaactgaaaaagatgaattaattttggaagttatgaacccacctcccaagaaaattcgactgcaagaactggaagatagtattgataatctaagtcaaaatggagagggaaggatctctgttagtcatgttggaagcactgcttccaagaactcaaatttaaatgtatgtaatgtatgcctaggaattcttcaagaattctgtgagaaagatttcattaaaaaggtgtgccaaaaggttgaggcctctgggtttgaattcaccagcttggtattttcagtctccttcccaccacaactatctgtaagagagcatgctgcatggttgctggtaaaacaggaaatgggaaagcagagtctgtcgctgggaagagatgatatagttcagctaaaagaagcctacaaatggataactcaccccctgttttcagaggaactgggtgttcccattgatggaaagagcttgtttgaagtgagtgtggtctttgctcacccagaaacagttgaggattgccacttcctagctgcgatatgcccagattgttttaagccagccaaaaacaaacagtctgtattcactagaatggcagttatgaaagccttgaataagataaaggaagaggatttccttaagcagtttccttgtcctccaaactcaccaaaggctgtatgcgctgttcttgaaattgaatgtgctcatggtgctgtttttgtggctgggagatataataaatactccaggaatctaccacaaactccttggataattgatggagaaaggaagctggaatcttcagtggaagaattaatttcagatcatctgttggcagtatttaaagcagagagttttaatttttcatcctctggaagagaagatgtagatgtgagaacattaggaaatggaaggccctttgcaattgagctggtgaatcctcatagagtacatttcacttcacaagaaattaaggaacttcagcagaaaattaataactcatctaacaaaatccaagtacgtgacttgcagcttgtcacaagagaggcaataggacatatgaaagaaggtgaagaagaaaagacaaagacctacagtgccttaatttggacaaataaagcgatacagaagaaagacattgaattcctaaatgacataaaggacttaaaaatcgaccagaaaacacctttgcgcgtccttcaccgaaggcccctggctgtgcgagctcgcgtcattcacttcatggagacacagtacgtggatgagcaccacttccgcctccacttgaaaactcaggctggcacctacattaaagagtttgtacatggagacttcgggagaaccaagccaaacattgggtccctgatgaatgtgactgcagacattctggagctggatgttgagtctgtagatgttgactggccacctgctctggatgac

SEQ ID NO: 16 - protein, 529 aa, Homo sapiens
MFPLTEENKHVAQLLLNTGTCPRCIFRFCGVDFHAPYKLPYKELLNELQKFLETEKDELILEVMNPPPKKIRLQELEDSIDNLSQNGEGRISVSHVGSTASKNSNLNVCNVCLGILQEFCEKDFIKKVCQKVEASGFEFTSLVFSVSFPPQLSVREHAAWLLVKQEMGKQSLSLGRDDIVQLKEAYKWITHPLFSEELGVPIDGKSLFEVSVVFAHPETVEDCHFLAAICPDCFKPAKNKQSVFTRMAVMKALNKIKEEDFLKQFPCPPNSPKAVCAVLEIECAHGAVFVAGRYNKYSRNLPQTPWIIDGERKLESSVEELISDHLLAVFKAESFNFSSSGREDVDVRTLGNGRPFAIELVNPHRVHFTSQEIKELQQKINNSSNKIQVRDLQLVTREAIGHMKEGEEEKTKTYSALIWTNKAIQKKDIEFLNDIKDLKIDQKTPLRVLHRRPLAVRARVIHFMETQYVDEHHFRLHLKTQAGTYIKEFVHGDFGRTKPNIGSLMNVTADILELDVESVDVDWPPALDD

SEQ ID NO: 17 - RNA, 1635 nt, Homo sapiens
  source 1..1635: mol_type = mRNA
atgtggctggaccgccgcggatggctcagggttcttggacattggcgctacgaccttaggcgccctagctttaccaggacttggagtggcgataagggcccaatggcagaaacagtgtctacccaggttgggacagagggcgggctgagggcttcgcatcagcaaaacggtgacgctggtggcgacgcgaaggttgagctgtcccccgggcccccgaagccggctggccgggaagtggagccggccccagtaggcggggagcatccctcggctgcagccccaggcccgggcaagcataagaagcggcggggcgcaaccagggagcgtgtcgtgccgcccccgaagaagcggcggaccggggtgagcttcggagatgagcactttgcagaaaccagttattacttcgagggcggcctgcgtaaggtgcggccctattactttgacttccggacctactgcaaaggtcgctgggtgggccacagcttgctgcacgtcttcagtaccgagttccgagctcagcccctggcctactatgaggccgcggtccgggcgggccgcctgcaactcaacgagaagccggtgcaggacctcaacatcgtgctcaaggacaatgatttcttgcggaacacagtgcacaggcatgagccaccagtcacagcagagcccattcgcctgctagctgagaacgaagatgtggtggttgtagacaagccttcctccattcccgttcacccctgtggccgcttccgacacaacacagttatcttcatcctaggcaaggagcaccaactcaaggagctacaccccttgcatcggcttgaccgccttacctcaggggtgcttatgtttgccaagacagctgcagtctctgagagaattcacgagcaggttcgggaccggcagctggagaaggagtacgtgtgccgggtggaaggggagttccccactgaggaagtgacctgtaaagaacccatcttagtggtgtcttacaaagtaggggtgtgccgtgtagatccccggggcaagccctgtgagacagtgttccagaggctaagctacaatggccagtccagtgtggtacggtgccggccactcacaggccgcacacaccagattcgagtccaccttcagttcttgggccatcccattctcaacgaccccatctacaactcagttgcctggggtccttctcgaggccggggcggctacattcccaagacaaacgaggagttgctacgggacctggtagcagagcaccaggccaaacagagcctggatgtgctagatctctgtgagggtgacctgtccccaggactcacagactctacggccccctcctcagagttgggcaaggacgacctggaagagttggctgcagctgcccagaagatggaggaagtagctgaggcagcccctcaggagttggacacaatagccttggcatcagagaaggcagttgaaacagatgtcatgaatcaagagacagacccactctgtgcagagtgccggctggtgcgacaggatcccttgccccaagaccttgtgatgttcctacatgccctacgctataaagggccaggctttgagtacttttcaccaatgcctgcctgggcacaggatgactggcaaaaggac

SEQ ID NO: 18 - protein, 545 aa, Homo sapiens
MWLDRRGWLRVLGHWRYDLRRPSFTRTWSGDKGPMAETVSTQVGTEGGLRASHQQNGDAGGDAKVELSPGPPKPAGREVEPAPVGGEHPSAAAPGPGKHKKRRGATRERVVPPPKKRRTGVSFGDEHFAETSYYFEGGLRKVRPYYFDFRTYCKGRWVGHSLLHVFSTEFRAQPLAYYEAAVRAGRLQLNEKPVQDLNIVLKDNDFLRNTVHRHEPPVTAEPIRLLAENEDVVVVDKPSSIPVHPCGRFRHNTVIFILGKEHQLKELHPLHRLDRLTSGVLMFAKTAAVSERIHEQVRDRQLEKEYVCRVEGEFPTEEVTCKEPILVVSYKVGVCRVDPRGKPCETVFQRLSYNGQSSVVRCRPLTGRTHQIRVHLQFLGHPILNDPIYNSVAWGPSRGRGGYIPKTNEELLRDLVAEHQAKQSLDVLDLCEGDLSPGLTDSTAPSSELGKDDLEELAAAAQKMEEVAEAAPQELDTIALASEKAVETDVMNQETDPLCAECRLVRQDPLPQDLVMFLHALRYKGPGFEYFSPMPAWAQDDWQKD

SEQ ID NO: 19 - DNA, 2898 bp, synthetic construct
  source 1..2898: mol_type = other DNA
atcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactggcgcactgggtggtcgctaacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcgccaacaaggccgtcgccctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattcc

SEQ ID NO: 20 - protein, 966 aa, synthetic construct
IEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 21 - RNA, 22 nt, synthetic construct
  source 1..22: mol_type = other RNA
ttaagtgacgaagaacaacttc

SEQ ID NO: 22 - RNA, 22 nt, synthetic construct
  source 1..22: mol_type = other RNA
aatttgatgagttctggcggct

SEQ ID NO: 23 - RNA, 22 nt, synthetic construct
  source 1..22: mol_type = other RNA
gttccttttccaagatgccctg

SEQ ID NO: 24 - RNA, 22 nt, synthetic construct
  source 1..22: mol_type = other RNA
attgtccagaacctgatctcta

SEQ ID NO: 25 - RNA, 22 nt, synthetic construct
  source 1..22: mol_type = other RNA
ggcactcttgcctacgccacca

SEQ ID NO: 26 - RNA, 23 nt, synthetic construct
  source 1..23: mol_type = other RNA
gatcatattcgtccacaaaatga

SEQ ID NO: 27 - RNA, 23 nt, synthetic construct
  source 1..23: mol_type = other RNA
gtacacaaagaaagccctcccca

SEQ ID NO: 28 - RNA, 23 nt, synthetic construct
  source 1..23: mol_type = other RNA
gttttttaattttcacacagcca

SEQ ID NO: 29 - RNA, 1179 nt, Homo sapiens
  source 1..1179: mol_type = mRNA
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagac

SEQ ID NO: 30 - RNA, 1179 nt, Homo sapiens
  source 1..1179: mol_type = mRNA
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacactttttgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagac

SEQ ID NO: 31 - RNA, 570 nt, Homo sapiens
  source 1..570: mol_type = mRNA
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatgtaa

SEQ ID NO: 32 - RNA, 567 nt, Homo sapiens
  source 1..567: mol_type = mRNA
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 33 - RNA, 570 nt, Homo sapiens; identical to SEQ ID NO: 31
  source 1..570: mol_type = mRNA

SEQ ID NO: 34 - RNA, 567 nt, Homo sapiens; identical to SEQ ID NO: 32
  source 1..567: mol_type = mRNA

SEQ ID NO: 35 - RNA, 27 nt, synthetic construct
  source 1..27: mol_type = other RNA
aaacgtttctgtcatccaaatactcca

SEQ ID NO: 36 - RNA, 137 nt, synthetic construct
  source 1..137: mol_type = other RNA
tcagccacgaaagcctgcacctgaccaggtctctgttggctggtgcaaccccagggtgagctgatagtaaaccccagctaatttgacagggttgttcttcatgtggatgactctgtctctaaagcatgggaacagcc

SEQ ID NO: 37 - RNA, 137 nt, synthetic construct
  source 1..137: mol_type = other RNA
tcagccacttagctgtgcacctgaccaggtctctgttggctggtgcaatcgtcaggtgagctgatagtaaaccccagctatcctcacagggttgttcttcatgtggatgactctgtttgttaagcatgggaacagcc

SEQ ID NO: 38 - RNA, 137 nt, synthetic construct
  source 1..137: mol_type = other RNA
tcagccactattatttgcacctgaccaggtctctgttggctggtgcaatggcaaggtgagctgatagtaaaccccagctttacatacagggttgttcttcatgtggatgactctgttttataagcatgggaacagcc

SEQ ID NO: 39 - protein, 8 aa, synthetic construct
SESATPES

SEQ ID NO: 40 - RNA, 137 nt, synthetic construct
  source 1..137: mol_type = other RNA
tcagccaccaagaactgcacctgaccaggtctctgttggctggtgcaatattgtggtgagctgatagtaaaccccagctgagggtacagggttgttcttcatgtggatgactctgtaggtgaagcatgggaacagcc

SEQ ID NO: 41 - RNA, 137 nt, synthetic construct
  source 1..137: mol_type = other RNA
tcagccacgaaagcctgcacctgaccaggtctctgttggctggtgcaaccccagggtgagctgatagtaaaccccagctaaagccacagggttgttcttcatgtggatgactctgtccccaaagcatgggaacagcc

SEQ ID NO: 42 - RNA, 137 nt, synthetic construct
  source 1..137: mol_type = other RNA
tcagccactctgaattgcacctgaccaggtctctgttggctggtgcaagctgtaggtgagctgatagtaaaccccagctctgaatacagggttgttcttcatgtggatgactctgtgctgtaagcatgggaacagcc

SEQ ID NO: 43 - RNA, 137 nt, synthetic construct
  source 1..137: mol_type = other RNA
tcagccacttagctgtgcacctgaccaggtctctgttggctggtgcaatcgtcaggtgagctgatagtaaaccccagcttagctgacagggttgttcttcatgtggatgactctgttcgtcaagcatgggaacagcc

SEQ ID NO: 44 - RNA, 137 nt, synthetic construct
  source 1..137: mol_type = other RNA
tcagccacaatcctctgcacctgaccaggtctctgttggctggtgcaattgttgggtgagctgatagtaaaccccagctatcctcacagggttgttcttcatgtggatgactctgtttgttaagcatgggaacagcc

SEQ ID NO: 45 - RNA, 137 nt, synthetic construct
  source 1..137: mol_type = other RNA
tcagccactattatttgcacctgaccaggtctctgttggctggtgcaatggcaaggtgagctgatagtaaaccccagctattattacagggttgttcttcatgtggatgactctgttggcaaagcatgggaacagcc